(19) United States
(12) Patent Application Publication
(10) Pub. No.: US 2008/0293679 A1
(43) Pub. Date: Nov. 27, 2008

(54) Use of carotenoids and/or carotenoid derivatives/analogs for reduction/inhibition of certain negative effects of COX inhibitors

(76) Inventors: Samuel F. Lockwood, Lago Vista, TX (US); R. Preston Mason, Manchester, MA (US)

Correspondence Address: MEYERTONS, HOOD, KIVLIN, KOWERT & GOETZEL, P.C., P.O. BOX 398, AUSTIN, TX, 78767-0398, US

(21) Appl. No.: 11/417,307

(22) Filed: May 2, 2006

Related U.S. Application Data
(60) Provisional application No. 60/699,717, filed on Jul. 15, 2005.
(60) Provisional application No. 60/718,450, filed on Sep. 19, 2005.

(51) Int. Cl. A61K 31/6615; A61K 31/122; A61K 31/015; A61K 31/047; A61K 31/01

(52) U.S. Cl. 514/102; 514/691; 514/762

(57) ABSTRACT

Administering carotenoids, and in particular xanthophyll carotenoids, or analogs or derivatives of astaxanthin, lutein, zeaxanthin, lycoxanthin, lycophyll, or lycopene to a subject undergoing treatment with COX-2 inhibitor drugs may reduce at least a portion of the adverse side effects associated with administration of COX-2 selective inhibitor drugs. The carotenoids, or analogs or derivatives thereof may be administered to a subject prior to, at the same time as, or after the commencement of COX-2 selective inhibitor drug therapy. The carotenoids, or analogs or derivatives thereof may be administered to a subject concurrently with COX-2 selective inhibitor drugs therapy. The carotenoids, or analogs or derivatives thereof may be incorporated into pharmaceutical preparation in combination with the COX-2 selective inhibitor drug or may be administered separately. Administration of the analogs or derivatives described herein may reduce peroxidation of LDL and other lipids in the serum and plasma cell membranes of subjects undergoing COX-2 selective inhibitor drug therapy. Administration of the analogs or derivatives described herein may reduce the incidence of deleterious clinical cardiovascular events undergoing COX-2 selective inhibitor drug therapy.

### CROSS-REFERENCE TO RELATED APPLICATIONS

The present application claims the benefit of priority under 35 U.S.C. §119 to U.S. Provisional Application No. 60/675,959, filed May 2, 2005; to U.S. Provisional Application No. 60/699,717, filed Jul. 15, 2005; and to U.S. Provisional Application No. 60/718,450, filed Sep. 19, 2005. The prior applications are considered part of the present application, and the contents thereof are hereby incorporated by reference in their entirety as though fully set forth herein.

### BACKGROUND OF THE INVENTION

1. Field of the Invention

The present invention generally relates to the fields of medicinal and synthetic chemistry. Specifically, the invention relates to the use of carotenoids, and in particular xanthophyll carotenoids, including analogs, derivatives, and intermediates thereof, as therapeutic agents that reduce or inhibit side effects associated with the administration of COX-2 selective inhibitors.

2. Description of the Related Art

Adverse Side Effects Associated with COX-2 Inhibitors

The development of selective inhibitors of the inducible cyclooxygenase-2 (COX-2) enzyme has been an important advance in the clinical management of pain associated with inflammatory disease, such as osteoarthritis. Unlike older non-steroidal antiinflammatory drugs (e.g., NSAIDs such as aspirin, indomethacin, ibuprofen, and naproxen), which inhibit both COX-2 and the constitutively expressed cyclooxygenase-1 (COX-1) enzyme, selective COX-2 inhibitor drugs relieve pain with minimal gastric erosion that can result from the inhibition of cytoprotective COX-1-dependent synthesis of prostaglandin E₂ (PGE₂) in the gastric mucosa. However, the recent finding that users of COX-2 selective inhibitor drugs are at significantly greater risk for the development of adverse cardiovascular events approximately 18 months after commencement of therapy has triggered the withdrawal from use of one of the most widely used COX-2 inhibitors, rofecoxib (Vioxx®). Early studies hypothesized that the cause of the adverse effects of COX-2 inhibitor drugs may be at least partially independent of their effects on the COX-2 enzyme. In support of this hypothesis, it has recently been demonstrated in vitro and in protein-free phospholipid systems that certain COX-2 selective inhibitor drugs can spontaneously oxidize and have pro-oxidant properties (Reddy and Corey, 2005; Walter et al, 2004). The presence of oxidized COX-2 selective inhibitor drugs was found to increase the production and levels of certain oxidized phospholipids, low density lipoprotein (LDL) and F₂-isprostanoids, the levels of which are correlated with the development of adverse cardiovascular conditions, such as atherosclerosis. Moreover, it was demonstrated that sulfone COX-2 inhibitor drugs could reduce the oxygen radical antioxidant capacity (ORAC) of human plasma. Although the magnitude of lipid peroxidation events (in particular the oxidative lag time of LDL cholesterol in the presence of a radical initiator) is somewhat reduced in the presence of a vitamin-E analog, the oxidation potential of COX-2 selective inhibitor drugs could not be fully reversed with this agent (Walter et al., 2004).

New methods of reducing or inhibiting one or more of the negative cardiovascular complications associated with therapeutic administration of COX-2 selective inhibitors in a subject would provide useful therapeutic agents.

### Antioxidant Properties of Carotenoids

Carotenoids are a group of natural pigments produced principally by plants, yeast, and microalgae. The family of related compounds now numbers greater than 750 described members, exclusive of Z and E isomers. Humans and other animals cannot synthesize carotenoids de novo and must obtain them from their diet. All carotenoids share common chemical features, such as a polyisoprenoid structure, a long polyene chain forming the chromophore, and near symmetry around the central double bond. Tail-to-tail linkage of two C₂₀ geranyl-geranyl diphosphate molecules produces the parent C₄₀ carbon skeleton. Carotenoids without oxygenated functional groups are called “carotenes”, reflecting their hydrocarbon nature; oxygenated carotenes are known as “xanthophylls.” “Parent” carotenoids may generally refer to those natural compounds utilized as starting scaffold for structural carotenoid analog synthesis. Carotenoid derivatives may be derived from a naturally occurring carotenoid. Naturally occurring carotenoids may include lycopene, lycophyll, lycoxanthin, astaxanthin, beta-carotene, lutein, zeaxanthin, and/or canthaxanthin to name a few.

Cyclization at one or both ends of the molecule yields 7 identified end groups (illustrative structures shown in FIG. 1). Examples of uses of carotenoid derivatives and analogs are illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

Documented carotenoid functions in nature include light-harvesting, photoprotection, and protective and sex-related coloration in microscopic organisms, mammals, and birds, respectively. A relatively recent observation has been the protective role of carotenoids against age-related diseases in humans as part of a complex antioxidant network within cells. This role is dictated by the close relationship between the physicochemical properties of individual carotenoids and their in vivo functions in organisms. The long system of alternating double and single bonds in the central part of the molecule (delocalizing the π-orbital electrons over the entire length of the polyene chain) confers the distinctive molecular shape, chemical reactivity, and light-absorbing properties of carotenoids. Additionally, isomerism around C═C double bonds yields distinctly different molecular structures that may be isolated as separate compounds (known as Z (“cis”) and E (“trans”), or geometric, isomers). Of the more than 750 described carotenoids, an even greater number of the theoretically possible mono-Z and poly-Z isomers are sometimes encountered in nature. The presence of a Z double bond creates greater steric hindrance between nearby hydrogen atoms and/or methyl groups, so that Z isomers are generally less stable thermodynamically, and more chemically reactive, than the corresponding all-E form. The all-E configuration is an extended, linear, and rigid molecule. Z-isomers are, by contrast, not simple, linear molecules (the so-called “bent-chain” isomers). The presence of any Z in the polyene chain creates a bent-chain molecule. The tendency of Z-isomers to crystallize or aggregate is much less than all-E, and Z isomers are more readily solubilized, absorbed, and transported in vivo than their all-E counterparts. This has important implications for enteral (e.g., oral) and parenteral (e.g., intravenous, intra-arterial, intramuscular, and subcutaneous) dosing in mammals.

Carotenoids with chiral centers may exist either as the R (rectus) or S (sinister) configurations. As an example, astaxanthin (with 2 chiral centers at the 3 and 3′ carbons) may exist as 4 possible stereoisomers: 3S,3′S; 3R,3′S and 3S,3′R (identical meso forms); or 3R,3′R. The relative proportions of each of the stereoisomers may vary by natural source. For example, Haematococcus pluvialis microalgal meal is 99% 3S,3′S astaxanthin, and is likely the predominant human evolutionary source of astaxanthin. Krill (3R,3′R) and yeast sources yield different stereoisomer compositions than the microalgal source. Synthetic astaxanthin, produced by large manufacturers such as Hoffmann-LaRoche AG, Buckton Scott (USA), or BASF AG, are provided as defined geometric isomer mixtures of a 1:2:1 stereoisomer mixture [3S,3′S; 3R,3′S, 3′R,3S (meso); 3R,3′R] of non-esterified, free astaxanthin. Natural source astaxanthin from salmonid fish is predominantly a single stereoisomer (3S,3′S), but does contain a mixture of geometric isomers. Astaxanthin from the natural source Haematococcus pluvialis may contain nearly 50% Z isomers. As stated above, the Z conformational change may lead to a higher steric interference between the two parts of the carotenoid molecule, rendering it less stable, more reactive, and more susceptible to reactivity at low oxygen tensions. In such a situation, in relation to the all-E form, the Z forms: (1) may be degraded first; (2) may better suppress the attack of cells by reactive oxygen species such as superoxide anion; and (3) may preferentially slow the formation of radicals. Overall, the Z forms may initially be thermodynamically favored to protect the lipophilic portions of the cell and the cell membrane from destruction. It is important to note, however, that the all-E form of astaxanthin, unlike β-carotene, retains significant oral bioavailability as well as antioxidant capacity in the form of its dihydroxy- and diketo-substitutions on the β-ionone rings, and has been demonstrated to have increased efficacy over β-carotene in most studies. The all-E form of astaxanthin has also been postulated to have the most membrane-stabilizing effect on cells in vivo. Therefore, it is likely that the all-E form of astaxanthin in natural and synthetic mixtures of stereoisomers is also extremely important in antioxidant mechanisms, and may be the form most suitable for particular pharmaceutical preparations.

The antioxidant mechanism(s) of carotenoids, and in particular astaxanthin, includes singlet oxygen quenching, direct radical scavenging, and lipid peroxidation chain-breaking. The polyene chain of the carotenoid absorbs the excited energy of singlet oxygen, effectively stabilizing the energy transfer by delocalization along the chain, and dissipates the energy to the local environment as heat. Transfer of energy from triplet-state chlorophyll (in plants) or other porphyrins and proto-porphyrins (in mammals) to carotenoids occurs much more readily than the alternative energy transfer to oxygen to form the highly reactive and destructive singlet oxygen (¹O₂). Carotenoids may also accept the excitation energy from singlet oxygen if any should be formed in situ, and again dissipate the energy as heat to the local environment. This singlet oxygen quenching ability has significant implications in cardiac ischemia, macular degeneration, porphyria, and other disease states in which production of singlet oxygen has damaging effects. In the physical quenching mechanism, the carotenoid molecule may be regenerated (most frequently), or be lost. Carotenoids are also excellent chain-breaking antioxidants, a mechanism important in inhibiting the peroxidation of lipids. Astaxanthin can donate a hydrogen (H.) to the unstable polyunsaturated fatty acid (PUFA) radical, stopping the chain reaction. Peroxyl radicals may also, by addition to the polyene chain of carotenoids, be the proximate cause for lipid peroxide chain termination. The appropriate dose of astaxanthin has been shown to completely suppress the peroxyl radical chain reaction in liposome systems. Astaxanthin shares with vitamin E this dual antioxidant defense system of singlet oxygen quenching and direct radical scavenging, and in most instances (and particularly at low oxygen tension in vivo) is superior to vitamin E as a radical scavenger and physical quencher of singlet oxygen.

Carotenoids, and in particular astaxanthin, are potent direct radical scavengers and singlet oxygen quenchers and possess all the desirable qualities of such therapeutic agents for inhibition or amelioration of ischemia-reperfusion (I/R) injury. Synthesis of novel carotenoid derivatives with “soft-drug” properties (i.e. activity in the derivatized form), with physiologically relevant, cleavable linkages to pro-moieties, can generate significant levels of free carotenoids in both plasma and solid organs. This is critically important, for in mammals, diesters of carotenoids generate the non-esterified or “free” parent carotenoid, and may be viewed as elegant synthetic and novel delivery vehicles with improved properties for delivery of free carotenoid to the systemic circulation and ultimately to target tissue. In the case of non-esterified, free astaxanthin, this is a particularly useful embodiment (characteristics specific to non-esterified, free astaxanthin below):
Lipid soluble in natural form; may be modified to become more water soluble
Molecular weight of 597 Daltons [size <600 daltons (Da) readily crosses the blood brain barrier, or BBB]
Long polyene chain characteristic of carotenoids effective in singlet oxygen quenching and lipid peroxidation chain breaking
No pro-vitamin A activity in mammals (eliminating concerns of hypervitaminosis A and retinoid toxicity in humans).

The administration of antioxidants which are potent singlet oxygen quenchers and direct radical scavengers, particularly of superoxide anion, should limit hepatic fibrosis and the progression to cirrhosis by affecting the activation of hepatic stellate cells early in the fibrogenetic pathway. Reduction in the level of ROS by the administration of a potent antioxidant can therefore be crucial in the prevention of the activation of both HSC and Kupffer cells. This protective antioxidant effect appears to be spread across the range of potential therapeutic antioxidants, including water-soluble (e.g., vitamin C, glutathione, resveratrol) and lipophilic (e.g., vitamin E, β-carotene, astaxanthin) agents. Therefore, a co-antioxidant derivative strategy in which water-soluble and lipophilic agents are combined synthetically is a particularly useful embodiment.

Vitamin E is generally considered the reference antioxidant. When compared with vitamin E, carotenoids are more efficient in quenching singlet oxygen in homogenenous organic solvents and in liposome systems. They are better chain-breaking antioxidants as well in liposomal systems. They have demonstrated increased efficacy and potency in vivo. They are particularly effective at low oxygen tension, and in low concentration, making them extremely effective agents in disease conditions in which ischemia is an important part of the tissue injury and pathology. These carotenoids also have a natural tropism for the liver after oral administration. Therefore, therapeutic administration of carotenoids should provide a greater benefit in limiting fibrosis than vitamin E.

Problems related to the use of some carotenoids and structural carotenoid analogs include: (1) the complex isomeric mixtures, including non-carotenoid contaminants, provided in natural and synthetic sources leading to costly increases in safety and efficacy tests required by such agencies as the FDA; (2) limited bioavailability upon administration to a subject; and (3) the differential induction of cytochrome P450 enzymes (this family of enzymes exhibits species-specific differences which must be taken into account when extrapolating animal work to human studies).

### SUMMARY OF THE INVENTION

In some embodiments, inhibiting, reducing or ameliorating at least some of the side effects associated with the administration of COX-2 inhibitors to a subject may include administering to the subject an effective amount of a pharmaceutically acceptable formulation including a xanthophyll or other carotenoid or a synthetic analog or derivative thereof. In an embodiment, the formulation may include astaxanthin, lutein and/or zeaxanthin. Negative consequences associated with therapeutic administration of COX-2-selective inhibitors that may be reduced by administering the xanthophyll carotenoids or synthetic derivatives or analogs thereof may include reducing lipid peroxidation and other measures of enzymatic and non-enzymatic oxidative stress, reducing radical flux, and reducing membrane de-stabilization caused by the presence of the COX-2 inhibitor.

In some embodiments, carotenoid analogs or carotenoid derivatives suited for use in the embodiments described herein include those derivatives or analogs that undergo chemical and/or enzymatic breakdown in a subject's body, in the digestive tract, in the serum, in the plasma, or in a cell, wherein at least one of the breakdown products is astaxanthin, or a derivative or an analog of astaxanthin.

In some embodiments, COX-2 selective inhibitors whose negative cardiovascular effects may be reduced may include sulfone-based COX-2 inhibitors such as, for example, rofecoxib and etoricoxib.

In some embodiments, COX-2 selective inhibitors whose negative cardiovascular effects may be reduced may include sulfonamide-based COX-2 inhibitors such as, for example, celecoxib, valdecoxib, or COX-2 selective inhibitors that are neither sulfone-nor sulfonamide-based, such as, for example, lumiracoxib.

In an embodiment, xanthophyll carotenoids or synthetic derivatives or analogs thereof may be administered to a subject concurrently with COX-2 selective inhibitor drug therapy, either as a co-formulation, or with separate pharmaceutical and/or nutraceutical agents. In an embodiment, xanthophyll carotenoids or synthetic derivatives or analogs thereof may be administered to a subject prior to the commencement of COX-2 selective inhibitor drug therapy. In an embodiment, xanthophyll carotenoids or synthetic derivatives or analogs thereof may be administered to a subject following the commencement of COX-2 selective inhibitor drug therapy.

Administration of xanthophyll carotenoids or a synthetic analogs or derivatives thereof according to the preceding embodiments may at least partially inhibit and/or influence the complications, particularly those complications associated with the cardiovascular system, associated with chronic administration of COX-2 selective inhibitor drugs such as rofecoxib, etoricoxib, celecoxib, and valdecoxib.

In some embodiments, the administration of carotenoids, xanthophyll carotenoids or structural analogs or derivatives of carotenoids by one skilled in the art—including consideration of the pharmacokinetics and pharmacodynamics of therapeutic drug delivery—is expected to inhibit and/or ameliorate disease conditions associated with administering xanthophyll carotenoid or a synthetic analog or derivative thereof to a subject, including but not limited to the production of oxidixed lipids, and LDL. In some of the foregoing embodiments, analogs or derivatives of carotenoids administered to cells may be at least partially water-soluble.

“Water-soluble” structural carotenoid analogs or derivatives are those analogs or derivatives that may be formulated in aqueous solution, either alone or with one or more excipients. Water-soluble carotenoid analogs or derivatives may include those compounds and synthetic derivatives that form molecular self-assemblies, and may be more properly termed “water dispersible” carotenoid analogs or derivatives. Water-soluble and/or “water-dispersible” carotenoid analogs or derivatives may be preferred in some embodiments.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/ml-10 mg/mL. In certain embodiments, water-soluble carotenoid analogs or derivatives may have a water solubility of greater than about 20 mg/mL. In certain embodiments, water-soluble carotenoid analogs or derivatives may have a water solubility of greater than about 25 mg/mL. In some embodiments, water-soluble carotenoid analogs or derivatives may have a water solubility of greater than about 50 mg/mL.

In some embodiments, water-soluble analogs or derivatives of carotenoids may be administered to a subject alone or in combination with additional xanthophyll carotenoids or structural analogs or derivatives. In some embodiments, water-soluble analogs or derivatives of carotenoids may be administered to a subject alone or in combination with other antioxidants.

In some embodiments, a method of inhibiting or reducing at least some of the side effects associated with therapeutic administration of COX-2 selective inhibitors may include administering to the subject an effective amount of a pharmaceutically acceptable formulation including a carotenoid. In some embodiments, a carotenoid may have the structure:

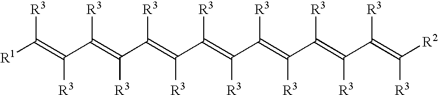

where each R³ is independently hydrogen or methyl, and where each R¹ and R² are independently:

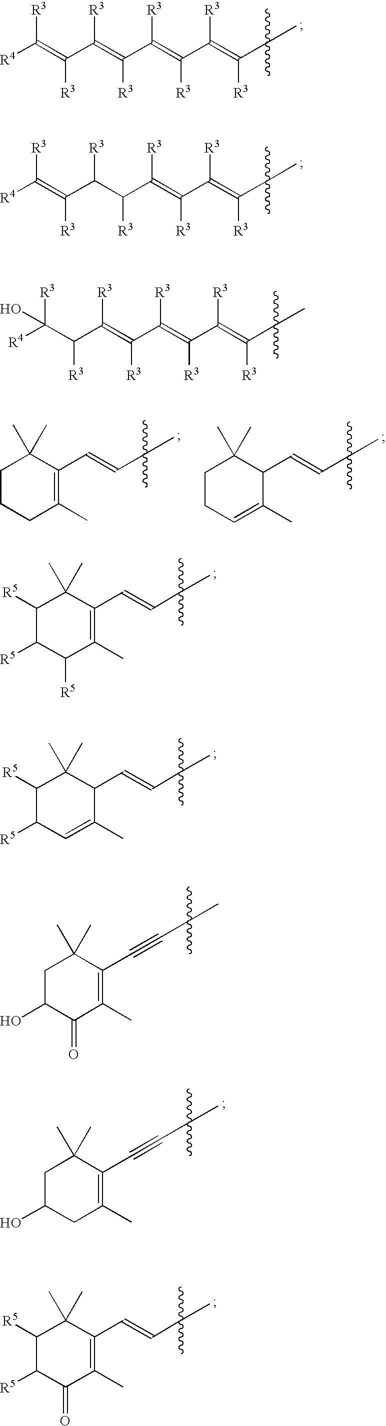

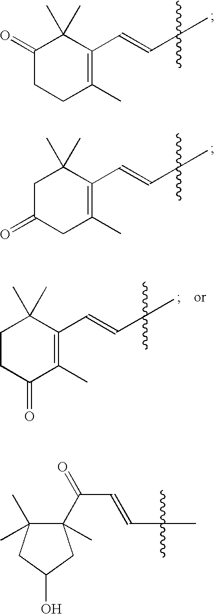

where R⁴ is hydrogen, methyl, or —CH₂OH; and where each R⁵ is independently hydrogen or —OH.

In some embodiments, a method of inhibiting or reducing at least some of the side effects associated with therapeutic administration of COX-2 selective inhibitors may include administering to the subject an effective amount of a pharmaceutically acceptable formulation including a synthetic analog or derivative of a carotenoid. The synthetic analog or derivative of the carotenoid may have the structure

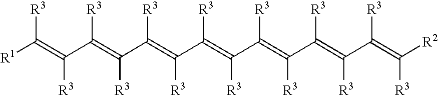

where each R³ is independently hydrogen or methyl, and where each R¹ and R² are independently:

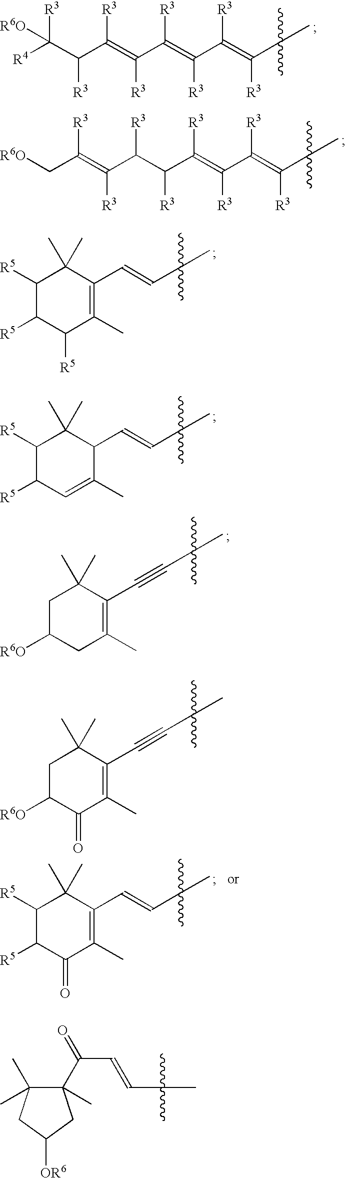

where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸; —P(O)(OR⁸)₂; —S(O)(OR⁸)₂; an amino acid; a peptide, a carbohydrate; —C(O)—(CH₂)ₙ—CO₂R⁹; —C(O)—OR⁹; a nucleoside residue, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with therapeutic administration of COX-2 selective inhibitors

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

In some embodiments, a pharmaceutical composition is provided that may include one or more carotenoids (“a co-formulation” strategy), or synthetic derivatives or analogs thereof, in combination with one or more COX-2 selective inhibitor drugs. Certain embodiments may further directed to pharmaceutical compositions that include combinations two or more carotenoids or synthetic analogs or derivatives thereof. In an embodiment, a pharmaceutical composition may include chiral astaxanthin in combination with a COX-2 selective inhibitor drug. In an embodiment, the COX-2 selective inhibitor drug may be a sulfone-based compound, such as rofecoxib or etoricoxib. In an embodiment, COX-2 selective inhibitor drug may be a sulfonamide-based compound such as celecoxib or valdecoxib. In an embodiment, a pharmaceutical composition suitable for use with the embodiments described herein may include at least one chiral astaxanthin, or a derivative or an analog thereof, and rofecoxib. The pharmaceutical compositions may be adapted to be administered orally, or by one or more parenteral routes of administration. In an embodiment, the pharmaceutical composition may be adapted such that at least a portion of the dosage of carotenoid or synthetic derivative or analog thereof is delivered prior to at least a portion of the COX-2 selective inhibitor drug being delivered.

In some embodiments, separate pharmaceutical compositions are provided, such that the COX-2 inhibitor is delivered separately from carotenoid, or synthetic derivatives or analogs thereof (sometimes referred to in the art as a “co-administration” strategy). The pharmaceutical compositions may be adapted to be administered orally, or by one or more parenteral routes of administration. In an embodiment, the pharmaceutical composition may be adapted such that at least a portion of the dosage of the carotenoid or synthetic derivative or analog thereof is delivered prior to, during, or after at least a portion of the COX-2 selective inhibitor drug is delivered to the subject.

Embodiments directed to pharmaceutical compositions may further include appropriate vehicles for delivery of said pharmaceutical composition to a desired site of action (i.e., the site a subject's body where the biological effect of the pharmaceutical composition is most desired). Pharmaceutical compositions including xanthophyll carotenoids or analogs or derivatives of astaxanthin, lutein or zeaxanthin that may be administered orally or intravenously may be particularly advantageous for and suited to embodiments described herein. In yet a further embodiment, an injectable astaxanthin formulation or a structural analog or derivative may be administered with a astaxanthin, zeaxanthin or lutein structural analog or derivative and/or other carotenoid structural analogs or derivatives, or in formulation with antioxidants and/or excipients that further the intended purpose. In some embodiments, one or more of the xanthophyll carotenoids or synthetic analogs or derivatives thereof may be at least partially water-soluble.

### BRIEF DESCRIPTION OF THE DRAWINGS

The above brief description as well as further objects, features and advantages of the methods and apparatus of the present invention will be more fully appreciated by reference to the following detailed description of presently preferred but nonetheless illustrative embodiments in accordance with the present invention when taken in conjunction with the accompanying drawings.

FIG. 1 depicts a graphic representation of several examples of the structures of several xanthophyll carotenoids and synthetic derivatives or analogs that may be used according to some embodiments. (A) astaxanthin; (B) lutein; (C) zeaxanthin; (D) disuccinic acid astaxanthin ester; (E) disodium disuccinic acid ester astaxanthin salt (Cardax™); and (F) divitamin C disuccinate astaxanthin ester; (G) tetrasodium diphosphate astaxanthin ester.

FIG. 2 depicts a time series of the UV/V is absorption spectra of the disodium disuccinate derivative of natural source lutein in water.

FIG. 3 depicts a UV/Vis absorption spectra of the disodium disuccinate derivative of natural source lutein in water (λₘₐₓ=443 nm), ethanol (λₘₐₓ=446 nm), and DMSO (λₘₐₓ=461 nm).

FIG. 4 depicts a UV/Vis absorption spectra of the disodium disuccinate derivative of natural source lutein in water (λₘₐₓ=442 nm) with increasing concentrations of ethanol.

FIG. 5 depicts a time series of the UV/Vis absorption spectra of the disodium diphosphate derivative of natural source lutein in water.

FIG. 6 depicts a UV/Vis absorption spectra of the disodium diphosphate derivative of natural source lutein in 95% ethanol (λₘₐₓ=446 nm), 95% DMSO (λₘₐₓ=459 nm), and water (λₘₐₓ=428 nm).

FIG. 7 depicts a UV/Vis absorption spectra of the disodium diphosphate derivative of natural source lutein in water (λₘₐₓ=428 nm) with increasing concentrations of ethanol.

FIG. 8 depicts a mean percent inhibition (±SEM) of superoxide anion signal as detected by DEPMPO spin-trap by the disodium disuccinate derivative of natural source lutein (tested in water).

FIG. 9 depicts a mean percent inhibition (±SEM) of superoxide anion signal as detected by DEPMPO spin-trap by the disodium diphosphate derivative of natural source lutein (tested in water).

FIG. 10A depicts comparative effects of NSAIDs onrates of conjugated diene formation in human LDL.

FIG. 10B depicts comparative effects of NSAIDs on formation of TBARS in human LDL.

FIG. 11A depicts the effects of rofecoxib and etoricoxib on isoprostane formation from lipid vesicles enriched with arachidonic acid.

FIG. 11B depicts comparative effects of COX-2 inhibitors on the antioxidant capacity of human plasma.

FIG. 12 depicts the effects of COX-2 inhibitors and carotenoids on lipid hydroperoxide formation in lipid vesicles enriched with arachidonic acid.

FIG. 13 depicts a schematic illustration of structure changes in a membrane with rofecoxib.

FIG. 14 depicts a summary of the cardiotoxic mechanisms for rofecoxib.

FIGS. 15A and 15B depict the effects of carotenoids on the structure of POPC membrane as different C/P ratios.

FIGS. 16A and 16B depict the effects of carotenoids on the membrane structure and peroxidation at different C/P ratios.

While the invention is susceptible to various modifications and alternative forms, specific embodiments thereof are shown by way of example in the drawings and will herein be described in detail. It should be understood, however, that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF THE PREFERRED EMBODIMENTS

### Definitions

The terms used throughout this specification generally have their ordinary meanings in the art, within the context of the invention, and in the specific context where each term is used. Certain terms are discussed below, or elsewhere in the specification, to provide additional guidance to the practitioner in describing the devices and methods of the invention and how to make and use them. It will be appreciated that the same thing can be said in more than one way. Consequently, alternative language and synonyms may be used for any one or more of the terms discussed herein, nor is any special significance to be placed upon whether or not a term is elaborated or discussed in greater detail herein. Synonyms for certain terms are provided. A recital of one or more synonyms does not exclude the use of other synonyms. The use of examples anywhere in this specification, including examples of any terms discussed herein, is illustrative only, and in no way limits the scope and meaning of the invention or of any exemplified term.

As used herein, the term “xanthophyll carotenoid” generally refers to a naturally occurring or synthetic 40-carbon polyene chain with a carotenoid structure that contains at least one oxygen-containing functional group. The chain may include terminal cyclic end groups. Exemplary, though non-limiting, xanthophyll carotenoids include astaxanthin, zeaxanthin, lutein, echinenone, lycophyll, canthaxanthin, and the like. Non-limiting examples of carotenoids that are not xanthophyll carotenoids include β-carotene and lycopene.

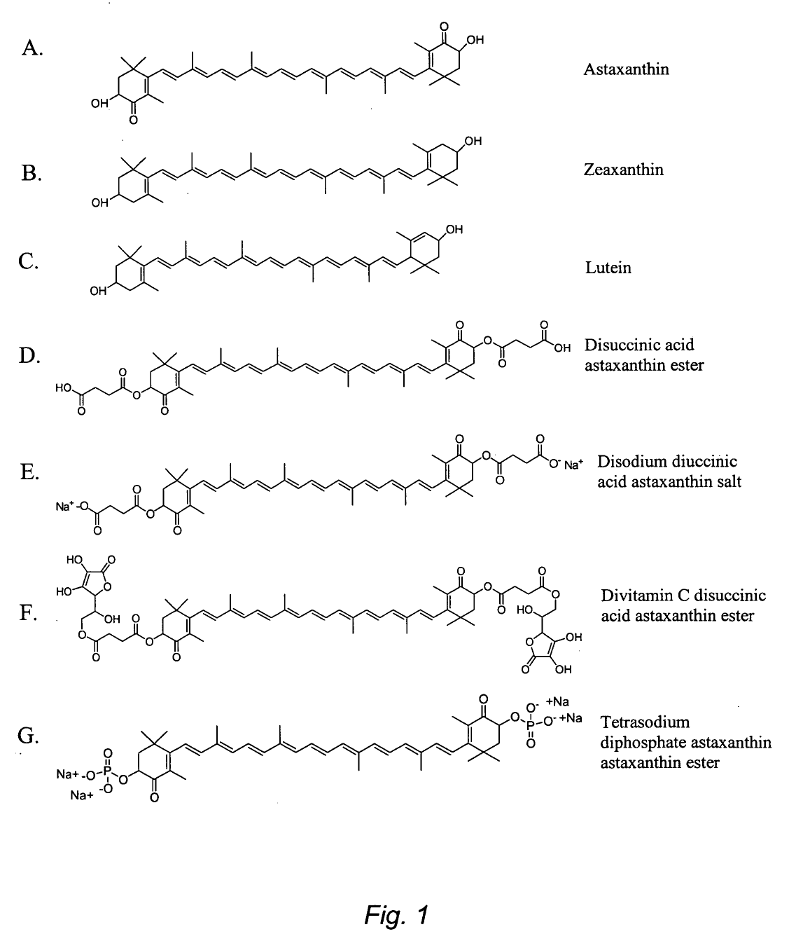

As used herein, terms such as “carotenoid analog” and “carotenoid derivative” generally refer to chemical compounds or compositions derived from a naturally occurring or synthetic carotenoid. Terms such as carotenoid analog and carotenoid derivative may also generally refer to chemical compounds or compositions that are synthetically derived from non-carotenoid based parent compounds; however, which ultimately substantially resemble a carotenoid derived analog. Non-limiting examples of carotenoid analogs and derivatives that may be used according to some of the embodiments described herein are depicted schematically in FIG. 1, D-G.

As used herein, the term “organ”, when used in reference to a part of the body of an animal or of a human generally refers to the collection of cells, tissues, connective tissues, fluids and structures that are part of a structure in an animal or a human that is capable of performing some specialized physiological function. Groups of organs constitute one or more specialized body systems. The specialized function performed by an organ is typically essential to the life or to the overall well-being of the animal or human. Non-limiting examples of body organs include the heart, lungs, kidney, ureter, urinary bladder, adrenal glands, pituitary gland, skin, prostate, uterus, reproductive organs (e.g., genitalia and accessory organs), liver, gall-bladder, brain, spinal cord, stomach, intestine, appendix, pancreas, lymph nodes, breast, salivary glands, lacrimal glands, eyes, spleen, thymus, bone marrow. Non-limiting examples of body systems include the respiratory, circulatory, cardiovascular, lymphatic, immune, musculoskeletal, nervous, digestive, endocrine, exocrine, hepato-biliary, reproductive, and urinary systems. In animals, the organs are generally made up of several tissues, one of which usually predominates, and determines the principal function of the organ.

As used herein, the term “tissue”, when used in reference to a part of a body or of an organ, generally refers to an aggregation or collection of morphologically similar cells and associated accessory and support cells and intercellular matter, including extracellular matrix material, vascular supply, and fluids, acting together to perform specific functions in the body. There are generally four basic types of tissue in animals and humans including muscle, nerve, epithelial, and connective tissues.

The term “modulate,” as used herein, generally refers to a change or an alteration in the magnitude of a be used herein to biological parameter such as, for example, foci formation, tumorigenic or neoplastic potential, apoptosis, growth kinetics, expression of one or more genes or proteins of interest, metabolism, oxidative stress, replicative status, intercellular communication, or the like. “Modulation” may refer to a net increase or a net decrease in the biological parameter.

As used herein the terms “reducing,” “inhibiting” and “ameliorating,” when used in the context of modulating a pathological or disease state, generally refers to the prevention and/or reduction of at least a portion of the negative consequences of the disease state. When used in the context of an adverse side effect associated with the administration of a drug to a subject, the term(s) generally refer to a net reduction in the severity or seriousness of said adverse side effects.

As used herein, the term “side effects associated with the administration of COX-2 inhibitors” generally refers to one or more unintended, although not necessarily unexpected, biological events associated with COX-2 inhibitor administration to a subject that are generally unrelated to the biological response(s) typically desired by said administration, namely, inhibition of the biological activity of the COX-2 enzyme in the subject. A side effect associated with the administration of COX-2 inhibitors is generally said to be “adverse” when the side effect can cause harm to the subject. Some of the adverse side effects associated with the administration of COX-2 inhibitors to a subject can occur systemically or at the level of one or more organ systems and include: increased incidences of edema, changes in systolic blood pressure that are greater than approximately 20 mm Hg, risk of stroke, risk of myocardial infarction, and increased risk of thrombotic events. Additional side effects that may occur at the level of biochemical reactions in an individual being administered COX-2 inhibitors include, by way of non-limiting example, the spontaneous development of pro-oxidant and toxic properties of the administered drug, increased production of certain oxidized phospholipids, LDL and F₂ isoprostanoids, reduced oxygen radical antioxidant capacity (ORAC), and increased lipid peroxidation events.

As used herein the term “COX-2 selective inhibitors” or “COX-2 inhibitor” generally refers to a compound belonging to a class of non-steroidal anti-inflammatory drugs (NSAIDs) that inhibit inducible cyclooxygenase (COX) enzymes, in particular COX-2. The term “COX-2 inhibitors” embraces those compounds that selectively inhibit cyclooxygenase-2 over cyclooxygenase-1. COX-2 and COX-1 inhibitory activities may be determined employing the human whole blood COX-1 assay and the human whole blood COX-2 assay described in C. Brideau et. Al., Inflamm. Res. 45: 68-74 (1996), herein incorporated by reference. The compounds may have a cyclooxygenase-2 IC₅₀ of less than about 2 μM in the human whole blood COX-2 assay, yet have a COX-1 IC₅₀ of greater than about 5 μM in the human whole blood COX-1 assay. Also, the compounds have a selectivity ratio of cyclooxygenase-2 inhibition over cyclooxygenase-1 inhibition of at least 5, or at least 30. “COX-2 selective inhibitor” may refer to sulfone drugs, such as rofecoxib or etoricoxib. COX-2 inhibitor may also refer to a sulfonamide drug such as celecoxib and valdecoxib. A variety of selective “COX-2 inhibitors” are known in the art. These include, but are not limited to, COX-2 inhibitors described in U.S. Pat. No. 5,474,995 “Phenyl heterocycles as COX-2 inhibitors”; U.S. Pat. No. 5,521,213 “Diaryl bicyclic heterocycles as inhibitors of cyclooxygenase-2”; U.S. Pat. No. 5,536,752 “Phenyl heterocycles as COX-2 inhibitors”; U.S. Pat. No. 5,550,142 “Phenyl heterocycles as COX-2 inhibitors”; U.S. Pat. No. 5,552,422 “Aryl substituted 5,5 fused aromatic nitrogen compounds as anti-inflammatory agents”; U.S. Pat. No. 5,604,253 “N-Benzylindol-3-yl propanoic acid derivatives as cyclooxygenase inhibitors”; U.S. Pat. No. 5,604,260 “5-Methanesulfonamido-1-indanones as an inhibitor of cyclooxygenase-2”; U.S. Pat. No. 5,639,780 N-Benzyl indol-3-yl butanoic acid derivatives as cyclooxygenase inhibitors”; U.S. Pat. No. 5,677,318 Diphenyl-1,2-3-thiadiazoles as anti-inflammatory agents”; U.S. Pat. No. 5,691,374 “Diaryl-5-oxygenated-2-(SH)-furanones as COX-2 inhibitors”; U.S. Pat. No. 5,698,584 “3,4-Diaryl-2-hydroxy-2,5-dihy-drofurans as prodrugs to COX-2 inhibitors”; U.S. Pat. No. 5,710,140 “Phenyl heterocycles as COX-2 inhibitors”; U.S. Pat. No. 5,733,909 “Diphenyl stilbenes as prodrugs to COX-2 inhibitors”; U.S. Pat. No. 5,789,413 “Alkylated styrenes as prodrugs to COX-2 inhibitors”; U.S. Pat. No. 5,817,700 “Bisaryl cyclobutenes derivatives as cyclooxygenase inhibitors”; U.S. Pat. No. 5,849,943 “Stilbene derivatives useful as cyclooxygenase-2 inhibitors”; U.S. Pat. No. 5,861,419 “Substituted pyridines as selective cyclooxygenase-2 inhibitors”; U.S. Pat. No. 5,922,742 “Pyridinyl-2-cyclopenten-1-ones as selective cyclooxygenase-2 inhibitors”; U.S. Pat. No. 5,925,631 “Alkylated styrenes as prodrugs to COX-2 inhibitors”; all of which are commonly assigned to Merck Frosst Canada, Inc. (Kirkland, Calif. or Merck & Co., Inc. (Rahway, N.J.). Additional COX-2 inhibitors are also described in U.S. Pat. Nos. 5,643,933, 6,001,843 “Substituted pyridines as selective cyclooxygenase-2 inhibitors”, U.S. Pat. No. 6,020,343 “(Methylsulfonyl)phenyl-2-(5H)-furanones as COX-2 inhibitors”, U.S. Pat. No. 5,409,944 “Alkanesulfonamido-1-indanone derivatives as inhibitors of cyclooxygenase”, U.S. Pat. No. 5,436,265 “1-aroyl-3-indolyl alkanoic acids and derivatives thereof useful as anti-inflammatory agents”, WO 94/15932, U.S. Pat. No. 5,344,991 “1,2 diarylcyclopentenyl compounds for the treatment of inflammation”, U.S. Pat. No. 5,134,142 “Pyrazole derivatives, and pharmaceutical composition comprising the same”, U.S. Pat. No. 5,380,738 “2-substituted oxazoles further substituted by 4-fluorophenyl and 4-methylsulfonylphenyl as antiinflammatory agents”, U.S. Pat. No. 5,393,790 “Substituted spiro compounds for the treatment of inflammation”, U.S. Pat. No. 5,466,823 “Substituted pyrazolyl benzenesulfonamides”, U.S. Pat. No. 5,633,272 “Substituted isoxazoles for the treatment of inflammation”, U.S. Pat. No. 5,932,598 “Prodrugs of benzenesulfonamide-containing COX-2 inhibitors”. A number of the aforementioned COX-2 inhibitors are pro-drugs of selective COX-2 inhibitors, and exert their action by conversion in vivo to the active and selective COX-2 inhibitors. The active and selective COX-2 inhibitors formed from the above-identified COX-2 inhibitor pro-drugs are described in detail in WO 95/00501, published Jan. 5, 1995, WO 95/18799, published Jul. 13, 1995 and U.S. Pat. No. 5,474,995, issued Dec. 12, 1995. Given the teachings of U.S. Pat. No. 5,543,297, entitled: “Human cyclooxygenase-2 cDNA and assays for evaluating cyclooxygenase-2 activity,” a person of ordinary skill in the art would be able to determine whether an agent is a selective COX-2 inhibitor or a precursor of a COX-2 inhibitor, and therefore part of the present embodiments. All of the above-identified patent or patent applications are hereby incorporated by reference as though fully set forth herein.

As used herein the terms “administration,” “administering,” or the like, when used in the context of providing a pharmaceutical or nutraceutical composition to a subject generally refers to providing to the subject one or more pharmaceutical, “over-the-counter” (OTC) or nutraceutical compositions in combination with an appropriate delivery vehicle by any means such that the administered compound achieves one or more of the intended biological effects for which the compound was administered. By way of non-limiting example, a composition may be administered parenteral, subcutaneous, intravenous, intracoronary, rectal, intramuscular, intra-peritoneal, transdermal, or buccal routes of delivery. Alternatively, or concurrently, administration may be by the oral route. The dosage administered will be dependent upon the age, health, weight, and/or disease state of the recipient, kind of concurrent treatment, if any, frequency of treatment, and/or the nature of the effect desired. The dosage of pharmacologically active compound that is administered will be dependent upon multiple factors, such as the age, health, weight, and/or disease state of the recipient, concurrent treatments, if any, the frequency of treatment, and/or the nature and magnitude of the biological effect that is desired.

As used herein, terms such as “pharmaceutical composition,” “pharmaceutical formulation,” “pharmaceutical preparation,” or the like, generally refer to formulations that are adapted to deliver a prescribed dosage of one or more pharmacologically active compounds to a cell, a group of cells, an organ or tissue, an animal or a human. Methods of incorporating pharmacologically active compounds into pharmaceutical preparations are widely known in the art. The determination of an appropriate prescribed dosage of a pharmacologically active compound to include in a pharmaceutical composition in order to achieve a desired biological outcome is within the skill level of an ordinary practitioner of the art. A pharmaceutical composition may be provided as sustained-release or timed-release formulations. Such formulations may release a bolus of a compound from the formulation at a desired time, or may ensure a relatively constant amount of the compound present in the dosage is released over a given period of time. Terms such as “sustained release,” “controlled release,” or “timed release” and the like are widely used in the pharmaceutical arts and are readily understood by a practitioner of ordinary skill in the art. Pharmaceutical preparations may be prepared as solids, semi-solids, gels, hydrogels, liquids, solutions, suspensions, emulsions, aerosols, powders, or combinations thereof. Included in a pharmaceutical preparation may be one or more carriers, preservatives, flavorings, excipients, coatings, stabilizers, binders, solvents and/or auxiliaries that are, typically, pharmacologically inert. It will be readily appreciated by an ordinary practitioner of the art that, included within the meaning of the term are pharmaceutically acceptable salts of compounds. It will further be appreciated by an ordinary practitioner of the art that the term also encompasses those pharmaceutical compositions that contain an admixture of two or more pharmacologically active compounds, such compounds being administered, for example, as a combination therapy.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylenediamine, diethylamine, 2-dibenzylethylenediamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

As used herein the terms “subject” generally refers to a mammal, and in particular to a human.

The terms “in need of treatment” or “in need thereof” when used in the context of a subject being administered a pharmacologically active composition, generally refers to a judgment made by an appropriate healthcare provider that an individual or animal requires or will benefit from a specified treatment or medical intervention. Such judgments may be made based on a variety of factors that are in the realm of expertise of healthcare providers, but include knowledge that the individual or animal is ill, will be ill, or is at risk of becoming ill, as the result of a condition that may be ameliorated or treated with the specified medical intervention.

By “therapeutically effective amount” is meant an amount of a drug or pharmaceutical composition that will elicit at least one desired biological or physiological response of a cell, a tissue, a system, animal or human that is being sought by a researcher, veterinarian, physician or other caregiver.

By “prophylactically effective amount” is meant an amount of a pharmaceutical composition that will substantially prevent, delay or reduce the risk of occurrence of the biological or physiological event in a cell, a tissue, a system, animal or human that is being sought by a researcher, veterinarian, physician or other caregiver.

The term “pharmacologically inert,” as used herein, generally refers to a compound, additive, binder, vehicle, and the like, that is substantially free of any pharmacologic or “drug-like” activity.

A “pharmaceutically or nutraceutically acceptable formulation,” as used herein, generally refers to a non-toxic formulation containing a predetermined dosage of a pharmaceutical and/or nutraceutical composition, wherein the dosage of the pharmaceutical and/or nutraceutical composition is adequate to achieve a desired biological outcome. The meaning of the term may generally include an appropriate delivery vehicle that is suitable for properly delivering the pharmaceutical composition in order to achieve the desired biological outcome.

As used herein the term “antioxidant” may be generally defined as any of various substances (as beta-carotene, vitamin C, and α-tocopherol) that inhibit oxidation or reactions promoted by Reactive Oxygen Species (ROS) and other radical and non-radical species.

As used herein the term “co-antioxidant” may be generally defined as an antioxidant that is used and that acts in combination with another antioxidant (e.g., two antioxidants that are chemically and/or functionally coupled, or two antioxidants that are combined and function with each another in a pharmaceutical preparation). The effects of co-antioxidants may be additive (i.e., the anti-oxidative potential of one or more anti-oxidants acting additively is approximately the sum of the oxidative potential of each component anti-oxidant) or synergistic (i.e., the anti-oxidative potential of one or more anti-oxidants acting synergistically may be greater than the sum of the oxidative potential of each component anti-oxidant).

Compounds described herein embrace isomers mixtures, racemic, optically active, and optically inactive stereoisomers and compounds.

### General Description

The presently described embodiments provide for a novel method for the treatment or prophylaxis of at least some of the adverse side effects associated with the administration of COX-2 inhibitors in patients comprising administering to said patients a therapeutically or prophylactically effective amount of a xanthophyll carotenoid, or a carotenoid derivative or analog, and/or a COX-2 selective inhibitor and a xanthophyll carotenoid, or a carotenoid derivative or analog.

The present invention also provides for pharmaceutical compositions comprising a therapeutically or prophylactically effective amount of a COX-2 inhibitor and a xanthophyll carotenoid, or a carotenoid derivative or analog, and a pharmaceutically acceptable carrier in unit dosage form.

The present invention also provides a pharmaceutical product comprising (1) a therapeutically or prophylactically effective amount of a COX-2 selective inhibitor in a first oral unit dosage form, (2) a xanthophyll carotenoid, or a carotenoid derivative or analog, in a second oral unit dosage form, and (3) instructions for concurrent or sequential administration of said pharmaceutical product to a patient in need thereof.

Clinical studies have demonstrated a correlation between the therapeutic use of certain cyclooxygenase-2 (COX-2) inhibitor drugs and increased risk for adverse cardiovascular events. This increased risk appears to be slightly more biased toward sulfone COX-2 selective inhibitor drugs such as refocoxib and etoricoxib than sulfonamide COX-2 inhibitors such as celecoxib and valdecoxib, suggesting that the mechanism by which the drugs increase the susceptibility of developing cardiovascular complication is independent of the ability of the drug to inhibit COX-2 enzyme function. While the mechanism by which COX-2 inhibitors exert adverse cardiovascular effects on subjects under treatment is not completely understood, emerging data suggest that sulfone COX-2 inhibitor class (e.g. rofecoxib and etoricoxib) exert non-enzymatic pro-oxidant effects in addition to the class effect that results from unopposed systemic inhibition of COX-2. The relative contribution of each of the aforementioned mechanisms is currently unclear, however, the net effect is that subjects under long term therapy with COX-2 inhibitors show an approximately a 5-fold increase in the incidence of clinical coronary events.

In vitro studies comparing the independent effects of COX-2 inhibitors on human LDL oxidation, an important contributor to atherosclerotic cardiovascular disease, showed that rofecoxib significantly decreases the lag time for LDL conjugated diene formation and increases the levels of thiobarbituric-acid-reactive-substances (TBARS). The pro-oxidant activity of COX-2 selective inhibitors appears to be dose-dependent and can be partially, though not fully, attenuated in vitro by a vitamin-E analog. Rofecoxib and etoricoxib can also cause a marked increase in non-enzymatic generation of isoprostanes. Addition of rofecoxib to fresh human plasma reduced the oxygen radical antioxidant capacity (ORAC) by 34% (p<0.0001).

Without being bound by any specific theory or mechanism of action, a physico-chemical basis for the pro-oxidant activity of certain COX-2 selective inhibitor drugs is provided by X-ray diffraction analyses, which indicate that sulfone COX-2 inhibitors interact with membrane phospholipids in a way that reduces the electron density of the hydrocarbon core of phospholipids, thus making them more susceptible to peroxidation by ROS (Walter et al., 2004). These studies demonstrate that sulfone COX-2 inhibitors increase the susceptibility of biological lipids to oxidative modification through a non-enzymatic process. These findings may provide mechanistic insight into reported differences in cardiovascular risk for COX-2 inhibitors. These studies are explained in detail in Walter et al. “Sulfone COX-2 inhibitors increase susceptibility of human LDL and plasma to oxidative modification: comparison to sulfonamide COX-2 inhibitors and NSAIDs”, Atherosclerosis 1 Vol. 77 (2004), pp. 235-243 which is incorporated by reference as though fully set forth herein. As discussed above, the extent to which the mechanisms governing the negative cardiovascular side effects of COX-2 enzymes are both non-enzymatic as well as enzymatic and constitute a class effect is not currently known. It is possible that at least a portion of the negative consequences described in Walter et al. 2004 and incorporated herein are extended to the other members of the COX-2 class of agents. Thus, therapeutic administration of the compounds and formulations described herein will be useful in treatment of these negative consequences of other COX-2 inhibitor drugs as well.

In some embodiments, a method is provided that substantially reduces at least a portion of the adverse cardiovascular side effects side effects associated with the administration of COX-2 inhibitor to a subject. The method may include administering to a subject in need thereof an effective amount of a pharmaceutically acceptable formulation that includes a xanthophyll carotenoid or a synthetic analog or derivative of a xanthophyll carotenoid. In an embodiment, the formulation may include astaxanthin, lutein and/or zeaxanthin or a structural analog or a derivative thereof. In an embodiment, the formulation may include homochiral (“chiral”) astaxanthin, or a synthetic analog or derivative of a homochiral astaxanthin. In an embodiment, the formulation may include mixtures of varying proporations of different homochiral forms of astaxanthin. In an embodiment, a synthetic analog or derivative of a chiral astaxanthin may be administered to a subject, wherein the synthetic chiral asatxanthin, when present in the subject's body, undergoes chemical or enzymatic breakdown, wherein at least one breakdown product is chiral asatxanthin. In certain preferred embodiments, the various synthetic and/or naturally occurring forms of astaxanthin (stereoisomers, geometric isomers, monoesters, diesters) may be administered to a subject to achieve the intended purpose. The term “astaxanthin” therefore includes its various chemical forms, and may include a certain preferred isomeric or ester form for a particular use. Exemplary though non-limiting xanthophyll carotenoids or structural derivatives or analogs thereof that may be suitable for use in the embodiments disclosed herein are depicted schematically in FIG. 1

In an embodiment, administering to a subject who is undergoing, who is expected to undergo, or who has undergone therapy with one or more COX-2 inhibitors may substantially inhibit or reduce the risk that the subject experiences adverse side effects associated with COX-2 inhibitors. In an embodiment, a subject may be administered xanthophyll carotenoids or structural derivatives or analogs thereof that are embodied herein to reduce the likelihood that the subject experiences edema, changes in systolic blood pressure that are greater than approximately 20 mm Hg, risk of stroke, myocardial infarction, or thrombotic events.

In an embodiment, a subject may be administered xanthophyll carotenoids or structural derivatives or analogs thereof embodied herein to inhibit or reduce certain adverse biochemical reactions, or complications arising from certain adverse biochemical reactions, that may occur in an individual being administered COX-2 inhibitors, such as, for example, the spontaneous development of prooxidant and toxic properties COX-2 inhibitors, increased production of certain oxidized phospholipids, LDL and F₂ isoprostanoids, reduced oxygen radical antioxidant capacity (ORAC), and increased lipid peroxidation events.

In an embodiment, administering a xanthophyll carotenoid or a structural derivative or an analog of a carotenoid may reduce the rate of lipid peroxidation resulting from the administration of COX-2 selective inhibitor drugs.

In some embodiments, COX-2 selective inhibitors whose negative cardiovascular effects may be reduced may include sulfone-based COX-2 inhibitors such as, for example, rofecoxib and etoricoxib.

In some embodiments, COX-2 selective inhibitors whose negative cardiovascular effects may be reduced may include sulfonamide-based COX-2 inhibitors such as, for example, celecoxib, valdecoxib, or COX-2 selective inhibitors that are neither sulfone-nor sulfonamide-based, such as, for example, lumiracoxib.

In an embodiment, carotenoids or synthetic derivatives or analogs thereof may be administered to a subject concurrently with COX-2 selective inhibitor drug therapy. In alternate embodiments, carotenoids or synthetic derivatives or analogs thereof may be administered to a subject prior to the commencement of COX-2 selective inhibitor drug therapy. In yet further embodiments, xanthophyll carotenoids or synthetic derivatives or analogs thereof may be administered to a subject following the commencement of COX-2 selective inhibitor drug therapy.

The carotenoids or synthetic derivatives or analogs thereof may be provided in a single pharmaceutical preparation together with a COX-2 selective inhibitor drug. Alternatively, the carotenoids or synthetic derivatives or analogs thereof may be provided may be administered to a subject in a pharmaceutical preparation that is distinct from that which includes the COX-2 selective inhibitor.

The pharmaceutical preparation may be administered orally, in the form of a tablet, a capsule, an emulsion, a liquid, or the like. Alternatively, the pharmaceutical preparation may be administered via a parenteral route. A more detailed description of the types of pharmaceutical preparations that may be suitable for some embodiments is described in below. Some embodiments may be particularly suited timed or sustained release pharmaceutical preparations, in which the preparation is adapted to deliver a known dosage of xanthophyll carotenoids or synthetic derivatives or analogs thereof at or over a predetermined time. In an embodiment, a pharmaceutical preparation may be adapted to one drug, or a portion thereof, before delivering the second drug. For example, a pharmaceutical preparation may be adapted in such a way that at least a portion of the xanthophyll carotenoid or structural analog or derivative thereof is released into the body of a subject before the COX-2 inhibitor drug is released. Such formulations may ensure that pro-oxidant capacity of COX-2 selective inhibitor drugs is maximally reduced.

In addition to carotenoids or structural analogs or derivatives thereof, a pharmaceutical composition may further include one or more co-antioxidant compounds. Exemplary, though non-limitive, co-antioxidant compounds that may be suitable for inclusion in a pharmaceutical preparation together with the xanthophyll carotenoids disclosed herein, or structural analogs or derivatives thereof, may include ascorbic acid or vitamin-E (α-tocopherol). The co-antioxidant compounds may improve the ability of xanthophyll carotenoids or structural derivatives or analogs thereof to reduce the pro-oxidant capacity of COX-2 selective inhibitor drugs. In an embodiment, co-antioxidant compounds may be covalently linked to the xanthophyll carotenoids or structural analogs or derivatives. Alternatively, co-antioxidant compounds may be mixed with the xanthophyll carotenoids or structural analogs or derivatives.

Administration of xanthophyll carotenoids or a synthetic analogs or derivatives thereof according to the preceding embodiments may at least partially inhibit and/or influence the complications, particularly those complications associated with the cardiovascular system, associated chronic administration of COX-2 selective inhibitor drugs such as rofecoxib, etoricoxib, celecoxib, and valdecoxib.

In some embodiments, the administration of xanthophyll carotenoids or structural analogs or derivatives of carotenoids by one skilled in the art—including consideration of the pharmacokinetics and pharmacodynamics of therapeutic drug delivery—is expected to inhibit and/or ameliorate disease conditions associated with administering xanthophyll carotenoid or a synthetic analog or derivative thereof to a subject, including but not limited to the production of oxidized lipids, isoprostanes and LDL. In some of the foregoing embodiments, analogs or derivatives of carotenoids administered to a subject may be adapted to be at least partially water-soluble.

In an embodiment, the xanthophyll carotenoids or structural carotenoid analogs or derivatives may at least partially counteract or reverse the ability of certain COX-2 selective inhibitor drugs to reduce the electron density of the hydrocarbon core of phospholipids. Restoring the electron density of the hydrocarbon core of phospholipids may render phospholipids present in cell membranes or LDL less susceptible to peroxidation by ROS. Reduced lipid peroxidation may, in turn, substantially prevent or reduce the occurrence of atherosclerosis or other adverse cardiovascular effects in subjects undergoing COX-selective inhibitor drug therapy. In an embodiment, the xanthophyll carotenoid or structural derivative or analog may be adapted or chosen such that the hydrophobic carotenoid backbone of the molecule incorporates into the plasma membrane of a cell in a plane that is substantially perpendicular the plane of the phospholipid bilayer. Such a configuration may allow the oxygen-containing functional groups of the xanthophyll carotenoid or structural analog or derivative thereof to anchor the molecule in a plane perpendicular to the phospholipids bilayer of the plasma membrane. Thus, in addition to providing potent antioxidant activity, a xanthophyll carotenoids or structural analogs or derivatives thereof may, at least in part, restore the electron density of the hydrocarbon core of phospholipids exposed to COX-2 selective inhibitor drugs. For such embodiments, a xanthophyll carotenoid or structural analog or derivative thereof may be selected such that the distance between the oxygen-containing function groups on opposing ends of the molecule is substantially similar to the thickness of the phospholipid bilayer of plasma membranes (e.g., between about 25 Å to about 55 Å, or between about 40 Å to about 50 Å). The inclusion of oxygen-containing function groups on opposing ends of the molecule may substantially prevent the molecule from adopting a configuration that is planar, and thus disruptive, to the plasma membrane. The inclusion of oxygen-containing function groups on opposing ends of the molecule may substantially prevent the molecule from adopting a configuration that is non-random, and thus also disruptive, to the plasma membrane.

### COX-2 Selective Inhibitors

As explained in J. Talley, Exp. Opin. Ther. Patents (1997), 7(1), pp. 55-62, three distinct structural classes of selective COX-2 inhibitor compounds have been identified. One class is the methane sulfonanilide class of inhibitors, of which NS-398, flosulide, nimesulide and (i) are example members.

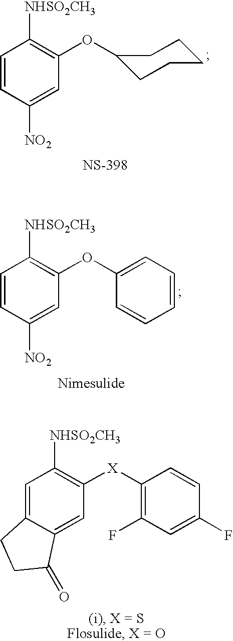

A second class is the tricyclic inhibitor class, which can be further divided into the sub-classes of tricyclic inhibitors with a central carbocyclic ring (examples include SC-57666, 1, and 2); those with a central monocyclic heterocyclic ring (examples include DuP 697, SC-58127, SC-58635, and 3, 4 and 5; and those with a central bicyclic heterocyclic ring (examples include 6, 7, 8, 9 and 10). Compounds 3, 4 and 5 are described in U.S. Pat. No. 5,474,995.

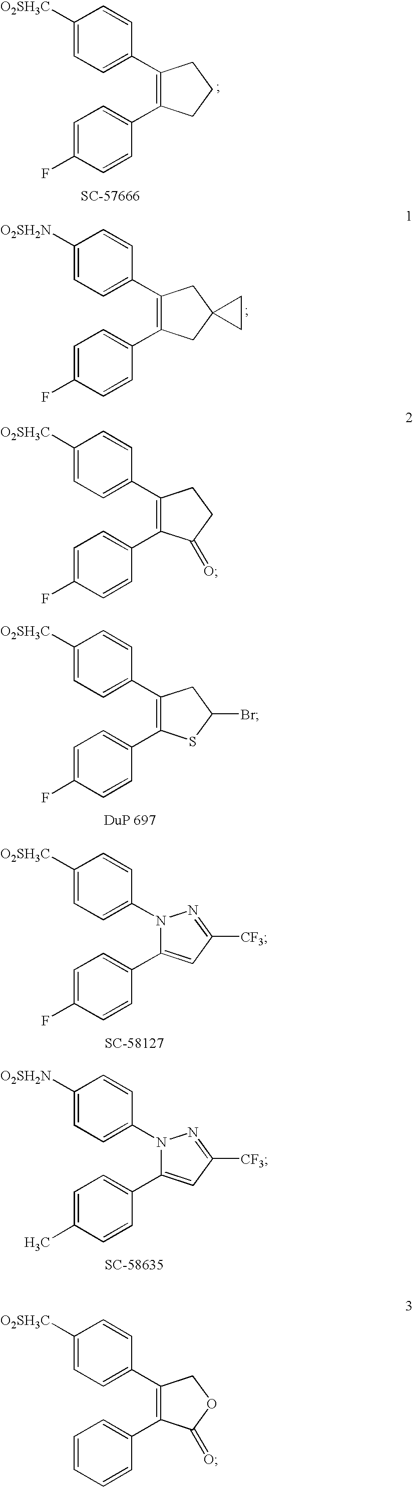

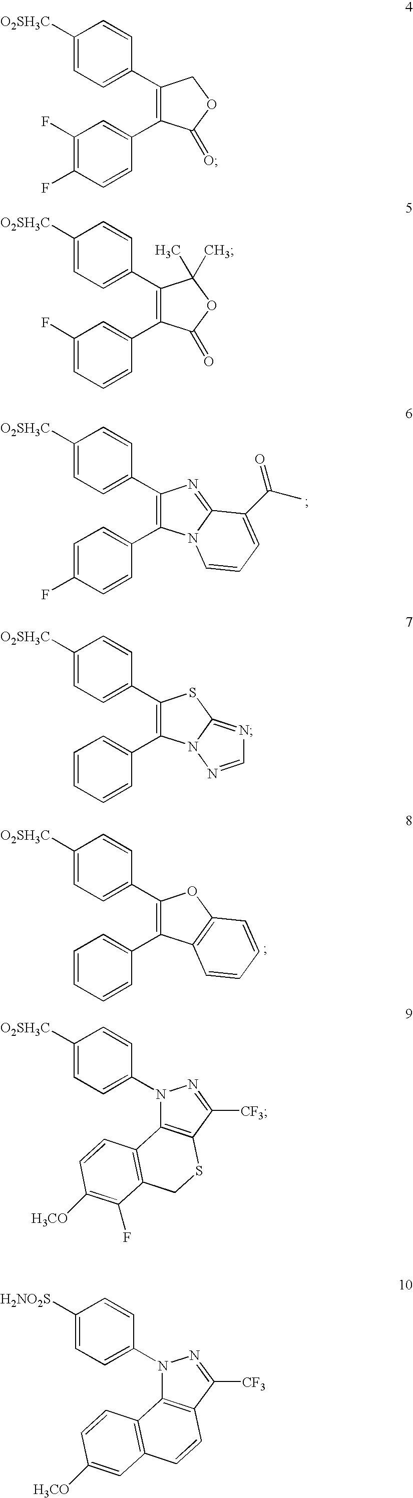

The third identified class can be referred to as those which are structurally modified NSAIDs, and includes 11a and structure 11 as example members.

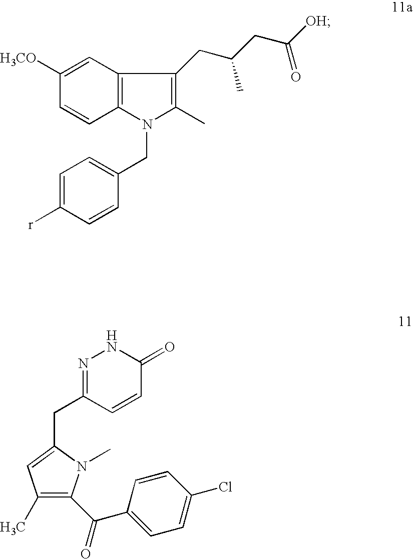

In addition to the structural classes, sub-classes, specific COX-2 selective inhibitor compound examples, and reference journal and patent publications described in the Talley publication which are all herein incorporated by reference, examples of compounds which selectively inhibit cyclooxygenase-2 have also been described in the following patent publications, all of which are herein incorporated by reference: U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780; and International Patent Specification Nos. 94/13635, 94/15932, 94/20480, 94/26731, 94/27980, 95/00501, 95/15316, 96/03387, 96/03388, 96/06840; and International Publication Nos. WO 94/20480, WO 96/21667, WO 96/31509, WO 96/36623, WO 97/14691, WO 97/16435.

One class of COX-2 inhibitors includes pyrazolyl-benzenesulfonamides. In one embodiment, methods of reducing the negative cardiovascular effects associated with therapeutic administration of pyrazolyl-benzenesulfonamides COX-2 inhibitors in a subject may include administering to the subject an effective amount of a pharmaceutically acceptable formulation including a xanthophyll or other carotenoid or a synthetic analog or derivative of a xanthophyll carotenoid and one or more pyrazolyl-benzenesulfonamides COX-2 inhibitors. Any of the carotenoids or carotenoid derivatives described herein may be used to reduce the negative cardiovascular effects associated with therapeutic administration of pyrazolyl-benzenesulfonamides COX-2 inhibitors.

Examples of pyrazolyl-benzenesulfonamides COX-2 inhibitors useful in treating inflammation and other COX-2 related disorders are defined by Formula I:

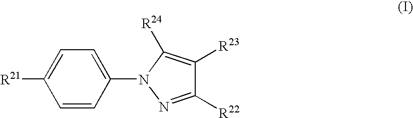

wherein R²¹ is selected from S(O)₂N(R²⁶)R²⁷, halo, alkyl, alkoxy, hydroxyl and haloalkyl;
wherein R²⁶ is hydrogen or alkoxycarbonylalkyl;
wherein R²⁷ is hydrogen, alkyl, carboxyalkyl, acyl, alkoxycarbonyl, heteroarylcarbonyl, alkoxycarbonylalkylcarbonyl, alkoxycarbonylcarbonyl, amino acid residue, or alkylcarbonylaminoalkylcarbonyl;
wherein R²² is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl and heterocyclic;
wherein R²³ is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl, heterocyclic, heterocycloalkyl and aralkyl;
wherein R²⁴ is selected from aryl, cycloalkyl, cycloalkenyl and heterocyclic; wherein R²⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy hydroxyalkyl haloalkoxy, sulfamyl, N-alkylsulfamyl, amino, N-alkylamino, N,N-dialkylamino, heterocyclic, nitro and acylamino; or wherein R²³ and R²⁴ together form:

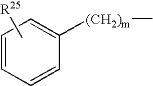

where m is 1 to 3, inclusive; and wherein R²⁵ is one or more radicals selected from halo, alkylthio, alkylsulfinyl, alkylsulfonyl, cyano, carboxyl, alkoxycarbonyl, amido, N-monoalkylamido, N-monoarylamido, alkyl, N,N-dialkylamido, N-alkyl-N-arylamido, haloalkyl, hydrido, hydroxyl, alkoxy, hydroxyalkyl, haloalkoxy, sulfamyl, N-alkylsulfamyl, amino, alkylamino, heterocyclic, nitro and acylamino; or a pharmaceutically-acceptable salt thereof. A description of the synthesis and use of pyrazolyl-benzenesulfonamides can be found in U.S. Pat. No. 5,466,823, which is incorporated herein by reference.

Pyrazolyl-benzenesulfonamides described herein would be useful for the treatment of inflammation in a subject, and for treatment of other inflammation-associated disorders, such as an analgesic in the treatment of pain and headaches, or as an antipyretic for the treatment of fever. For example, pyrazolyl-benzenesulfonamides described herein would be useful to treat arthritis, including but not limited to rheumatoid arthritis, spondyloarthopathies, gouty arthritis, systemic lupus erythematosus, osteoarthritis and juvenile arthritis. Such pyrazolyl-benzenesulfonamides described herein would be useful in the treatment of asthma, bronchitis, menstrual cramps, tendinitis, bursitis, and skin-related conditions such as psoriasis, eczema, burns and dermatitis.

A sub-class of pyrazolyl-benzenesulfonamides includes those compounds having the structure shown in Formula II below:

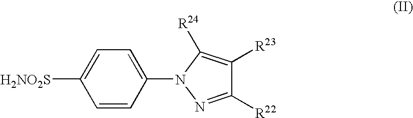

wherein R²² is haloalkyl; wherein R²³ is hydrido; and wherein R²⁴ is selected from aryl, cycloalkyl, and cycloalkenyl;
wherein R²⁴ is optionally substituted at a substitutable position with one or more radicals selected from halo, alkylthio, alkylsulfonyl, cyano, nitro, haloalkyl, alkyl, hydrido, alkoxy, haloalkoxy, sulfamyl, heterocyclic and amino;
or a pharmaceutically-acceptable salt thereof.

A specific example of a pyrazolyl-benzenesulfonamide includes the compound depicted in formula III below:

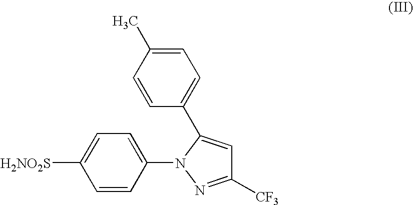

Another class of COX-2 inhibitors includes isoxazoles. In one embodiment, methods of reducing the negative cardiovascular effects associated with therapeutic administration of isoxazole COX-2 inhibitors in a subject may include administering to the subject an effective amount of a pharmaceutically acceptable formulation including a xanthophyll or other carotenoid or a synthetic analog or derivative of a xanthophyll carotenoid and one or more isoxazole COX-2 inhibitors. Any of the carotenoids or carotenoid derivatives described herein may be used to reduce the negative cardiovascular effects associated with therapeutic administration of isoxazole COX-2 inhibitors.

Examples of isoxazole COX-2 inhibitors useful in treating inflammation and other COX-2 related disorders are defined by Formula IV:

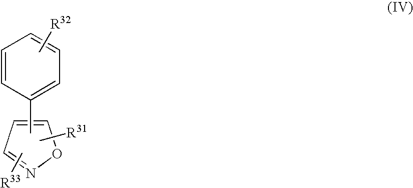

wherein R³¹ is selected from R—, RO—, RS—, RO-alkyl, RS-alkyl, carboxyl, cyano, hydroxyl, amino, halo, carboxyalkyl, alkoxycarbonylalkyl, aminocarbonyl, aminocarbonylalkyl, alkoxyalkyloxyalkyl, aryl(hydroxylalkyl), haloalkylsulfonyloxy, arylcarbonyloxyalkyl, arylcarbonylthioalkyl, alkoxycarbonyloxyalkyl, alkylaminocarbonyloxyalkyl, alkylaminocarbonylthioalkyl, RS(O)—; RS(O)alkyl-; RC(O)—; RC(O)alkyl-; ROC(O)—; ROC(O)alkyl-; RNH—; RNHalkyl-; R₂N—; R₂Nalkyl-; RS(O)₂alkyl-; and R^{a}O₂CR^{b}—X-alkyl-; wherein R is independently selected from alkyl, haloalkyl, hydroxyalkyl, aryl, cycloalkyl, heterocyclo, aralkyl, cycloalkylalkyl, and heterocycloalkyl; wherein R^{a} is selected from hydrido and R; wherein R^{b} is selected from a direct bond, alkyl, haloalkyl, hydroxyalkyl, aryl, cycloalkyl, heterocyclo, alkylaryl, aralkyl, cycloalkylalkyl, and heterocycloalkyl; wherein X is selected from O, S and S(O); wherein R³² is S(O)₂N(R³⁹)R⁴⁰; wherein R³⁹ is hydrogen or alkoxycarbonylalkyl; wherein R⁴⁰ is hydrogen, alkyl, carboxyalkyl, acyl, alkoxycarbonyl, heteroarylcarbonyl, alkoxycarbonylalkylcarbonyl, alkoxycarbonylcarbonyl, amino acid residue, or alkylcarbonylaminoalkylcarbonyl; and wherein R³³ is selected from cycloalkyl, cycloalkenyl, aryl and heterocyclo; wherein R³³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; or a pharmaceutically-acceptable salt thereof A description of the synthesis and use of suitable isoxazole COX-2 inhibitors can be found in U.S. Pat. Nos. 5,932,598, 5,859,257 and 5,633,272, all of which are incorporated herein by reference.

Compounds of Formula IV would be useful for, but not limited to, the treatment of inflammation in a subject, and for treatment of other cyclooxygenase-2 mediated disorders, such as, as an analgesic in the treatment of pain and headaches, or as an antipyretic for the treatment of fever. For example, compounds of the invention would be useful to treat arthritis, including but not limited to rheumatoid arthritis, spondyloarthropathies, gouty arthritis, osteoarthritis, systemic lupus erythematosus and juvenile arthritis.

A sub-class of isoxazole COX-2 inhibitors includes those compounds defined by Formula V:

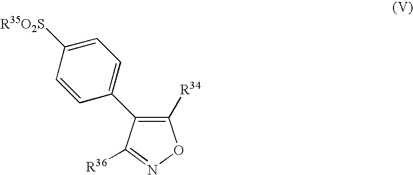

wherein R³⁴ is selected from hydroxyl, alkyl, carboxyl, halo, carboxyalkyl, alkoxycarbonylalkyl, aralkyl, methoxy, ethoxy, butoxy, alkylthio, alkoxyalkyl, aryloxyalkyl, arylthioalkyl, haloalkyl, hydroxylalkyl, aralkoxyalkyl, aryl(hydroxylalkyl), carboxyalkoxyalkyl, carboxyaryloxyalkyl, alkoxycarbonylaryloxyalkyl, cycloalkyl and cycloalkylalkyl; wherein R³⁵ is N(R³⁹)R⁴⁰; wherein R³⁹ is hydrogen; wherein R⁴⁰ is hydrogen, alkyl or —C(O)alkyl; and wherein R³⁶ is phenyl; wherein R³⁶ is optionally substituted at a substitutable position with one or more radicals independently selected from alkylsulfinyl, alkyl, cyano, carboxyl, alkoxycarbonyl, haloalkyl, hydroxyl, hydroxyalkyl, amino, haloalkoxy, alkylamino, phenylamino, aminoalkyl, nitro, halo, alkoxy, methylenedioxy, aminosulfonyl, and alkylthio; or a pharmaceutically-acceptable salt thereof.

A further sub-class of isoxazole COX-2 inhibitors includes those compounds defined by Formula VI:

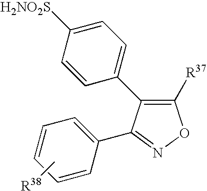

wherein R³⁷ is selected from hydroxyl, alkyl, carboxyl, halo, carboxyalkyl, alkoxycarbonylalkyl, alkoxyalkyl, carboxyalkoxyalkyl, haloalkyl, alkylthio, alkylsulfinyl, (hydroxy)alkoxyalkyl, carboxyalkylaryloxyalkyl, haloalkylsulfonyloxy, hydroxylalkyl, aryl(hydroxylalkyl), carboxyaryloxyalkyl, cycloalkyl, cycloalkylalkyl, and aralkyl; and wherein R³⁸ is one or more radicals independently selected from hydrido, alkylsulfinyl, alkyl, cyano, carboxyl, alkoxycarbonyl, haloalkyl, hydroxyl, hydroxyalkyl, haloalkoxy, amino, alkylamino, arylamino, aminoalkyl, nitro, halo, alkoxy, aminosulfonyl, and alkylthio; or a pharmaceutically-acceptable salt thereof. Specific examples of compounds having the structure defined by Formula VI include:

4-[5-ethyl-3-phenylisoxazol-4-yl]benzenesulfonamide; 4-[3-phenyl-5-propylisoxazol-4-yl]benzenesulfonamide; 4-[5-isopropyl-3-phenylisoxazol-4-yl]benzenesulfonamide; 4-[5-butyl-3-phenylisoxazol-4-yl]benzenesulfonamide; 4-[5-isobutyl-3-phenylisoxazol-4-yl]benzenesulfonamide; 4-[5-cyclohexyl-3-phenylisoxazol-4-yl]benzenesulfonamide; 4-[5-neopentyl-3-phenylisoxazol-4-yl]benzenesulfonamide; 4-[5-cyclohexylmethyl-3-phenylisoxazol-4-yl]benzenesulfonamide; 4-[5-(4-chlorophenyl)methyl-3-phenylisoxazol-4-yl]benzenesulfonamide; 4-[5-difluoromethyl-3-phenylisoxazol-4-yl]benzenesulfonamide; 4-[5-chloromethyl-3-phenylisoxazol-4-yl]benzenesulfonamide; 4-[5-methoxymethyl-3-phenylisoxazol-4-yl]benzenesulfonamide; 4-[5-(3-hydroxypropyl)-3-phenylisoxazol-4-yl]benzenesulfonamide; 4-[3-(4-chlorophenyl)-5-methyl-isoxazol-4-yl]benzenesulfonamide; 4-[3-(4-fluorophenyl)-5-methyl-isoxazol-4-yl]benzenesulfonamide; 4-[3-(3-fluoro-4-methylphenyl)-5-methyl-isoxazol-4-yl]benzenesulfonamide; 4-[3-(3-chloro-4-methylphenyl)-5-methyl-isoxazol-4-yl]benzenesulfonamide; 4-[3-(3-fluorophenyl)-5-methyl-isoxazol-4-yl]benzenesulfonamide; 4-[5-hydroxymethyl-3-phenylisoxazol-4-yl]benzenesulfonamide; [4-[4-(aminosulfonyl)phenyl]-3-phenylisoxazol-5-yl]carboxylic acid; 4-[5-hydroxy-3-phenyl-4-isoxazolyl]benzenesulfonamide; 4-[5-methyl-3-phenyl-isoxazol-4-yl]benzenesulfonamide; 4-[3-(3-fluoro-4-methoxyphenyl)-5-methyl-isoxazol-4-yl]benzenesulfonamide; 4-[3-phenyl-5-(3,3,3-trifluoro-2-oxopropyl)isoxazol-4-yl]benzenesulfonamide; [4-[4-(aminosulfonyl)phenyl]-3-phenyl-isoxazol-5-yl]acetic acid; [4-[4-(aminosulfonyl)phenyl]-3-phenyl-isoxazol-5-yl]propanoic acid; ethyl [4-[4-(aminosulfonyl)phenyl]-3-phenyl-isoxazol-5-yl]propanoate; [4-[4-(aminosulfonyl)phenyl]-3-(3-fluoro-4-methoxyphenyl)isoxazol-5-yl]propanoic acid and 4-[5-methyl-3-phenylisoxazol-4-yl]benzenesulfonamide.

A specific example of an isoxazole COX-2 inhibitor includes the compound depicted in formula VII below:

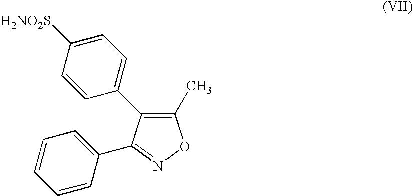

Another class of COX-2 inhibitors includes various phenyl heterocycles. In one embodiment, methods of reducing the negative cardiovascular effects associated with therapeutic administration of phenyl heterocycle COX-2 inhibitors in a subject may include administering to the subject an effective amount of a pharmaceutically acceptable formulation including a xanthophyll or other carotenoid or a synthetic analog or derivative of a xanthophyll carotenoid and one or more phenyl heterocycle COX-2 inhibitors. Any of the carotenoids or carotenoid derivatives described herein may be used to reduce the negative cardiovascular effects associated with therapeutic administration of phenyl heterocycle COX-2 inhibitors.

Examples of phenyl heterocycle COX-2 inhibitors useful in treating inflammation and other COX-2 related disorders are defined by Formula VIII or pharmaceutically acceptable salts of the compounds defined by Formula VIII:

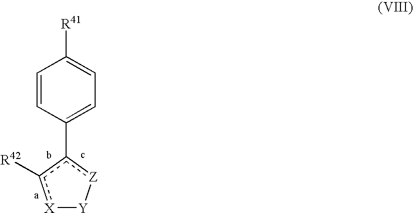

wherein: X-Y-Z-is: (a) —CH₂CH₂CH₂—; (b) —C(O)CH₂CH₂—; (c) —CH₂CH₂C(O)—;

### (d) —CR⁴⁵(R⁴⁵)—O—C(O)—; (e) —C(O)—O—CR⁴⁵(R^{45′})—; (f) —CH₂—NR₃—CH₂—;

### (g) —CR⁴⁵(R^{45′})—NR₃—C(O)—; (h) —CR⁴⁴═CR^{44′}—S—; (i) —S—CR⁴⁴═CR^{44′}—; (j) —S—N═CH—;

### (k) —CH═N—S—; (l) —N═CR⁴⁴—O—; (m) —O—CR⁴⁴═N—; (n) —N═CR⁴⁴—NH—;

### (o) —N═CR⁴⁴—S—; (p) —S—CR⁴⁴—N—; (q) —C(O)—NR⁴³—CR⁴⁵(R^{45′})—; (r) —R⁴³N—CH═CH—; or

(s) —CH═CH—NR⁴³— when side b is a double bond, and sides a an c are single bonds; and

### X-Y-Z-is: (a) ═CH—O—CH═; (b) ═CH—NR⁴³—CH═; (c) ═N—S—CH═; (d) ═CH—S—N═;

(e) ═N—O—CH═; (f) ═CH—O—N═; (g) ═N—S—N═; or (h) ═N—O—N═ when sides a and c are double bonds and side b is a single bond;
where R⁴¹ is: (a) S(O)₂CH₃; (b) S(O)₂NH₂; (c) S(O)₂NHC(O)CF₃; (d) S(O)(NH)CH₃;
(e) S(O)(NH)NH₂; (f) S(O)(NH)NHC(O)CF₃; (g) P(O)(CH₃)OH; (h) P(O)(CH₃)NH₂; (i) S(O)₂NH-alkyl; (j) S(O)₂NH-aryl; (k) S(O)₂NHC(O)-alkyl; or (l) S(O)₂NHC(O)aryl;
where R⁴² is (a) C₁₋₆ alkyl; (b) C₃, C₄, C₅, C₆, or C₇ cycloalkyl; (c) mono-, di- or tri-substituted phenyl or naphthyl wherein possible substituents include hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R⁴⁵)(R⁴⁶)—OH, —C(R⁴⁵)(R⁴⁶)—O—C, alkyl, and (13) —C₁₋₆ alkyl-CO₂—R⁴⁵; (d) mono-, di- or tri-substituted heteroaryl wherein the heteroaryl is a monocyclic aromatic ring of 5 atoms, the monocyclic ring having one heteroatom which is S, O, or N, and optionally 1, 2, or 3 additionally N atoms; or the heteroaryl is a monocyclic ring of 6 atoms, the monocyclic ring having one heteroatom which is N, and optionally 1, 2, 3, or 4 additional N atoms; wherein possible substituents include hydrogen, halo (including fluoro, chloro, bromo and iodo), C₁₋₆ alkyl, C₁₋₆ alkoxy, C₁₋₆ alkylthio, CN, CF₃, N₃, —C(R⁴⁵)(R⁴⁶)—OH, and —C(R⁴⁵)(R⁴⁶)—O—C₁₋₄ alkyl; or (e) benzoheteroaryl which includes the benzo fused analogs of (d);
where each R⁴³ is: (a) hydrogen; (b) CF₃; (c) CN; (d) C₁₋₆ alkyl; (e) hydroxy C₁₋₄ alkyl;
(f) —C(O)—C₁₋₆ alkyl; (g) optionally substituted (1) —C₁₋₅ alkyl-Q,
(2) —C₁₋₃ alkyl-O—C₁₋₃ alkyl-Q, (3) —C₁₋₃ alkyl-S—C₁₋₃ alkyl-Q,
(4) —C₁₋₅ alkyl-O-Q, or (5) —C₁₋₅ alkyl-S-Q, wherein the substituent resides on the alkyl and the substituent is C₁₋₃ alkyl; or (h) -Q
where R⁴⁴ and R^{44′} are each independently: (a) hydrogen; (b) CF₃; (c) CN; (d) C₁₋₆ alkyl; (e) -Q; (f) —O-Q; (g) —S-Q, or (h) optionally substituted (1) —C₁₋₅ alkyl-Q, (2) —O—C₁₋₅ alkyl-Q, (3) —S—C₁₋₅ alkyl-Q, (4) —C₁₋₃-alkyl-O—C₁₋₃ alkyl-Q, (5) —C₁₋₃ alkyl-S—C₁₋₃ alkyl-Q, (6) —C₁₋₅ alkyl-O-Q, (7) —C₁₋₅ alkyl-S-Q, wherein the substituent resides on the alkyl and the substituent is C₁₋₃ alkyl, and
where R⁴⁵, R^{45′}, R⁴⁶, R⁴⁷ and R⁴⁸ are each independently: (a) hydrogen; (b) C₁₋₆ alkyl; or R⁴⁵ and R⁴⁶ or R⁴⁷ and R⁴⁸ together with the carbon to which they are attached form a saturated monocyclic carbon ring of 3, 4, 5, 6 or 7 atoms;
where Q is CO₂H, CO₂—C₁₋₄ alkyl, tetrazolyl-5-yl, C(R⁴⁷)(R⁴)(OH), or C(R⁴⁷)(R⁴⁸)(O—C₁₋₄ alkyl).

A description of the synthesis and use of suitable phenyl heterocycle COX-2 inhibitors can be found in U.S. Pat. No. 5,474,995, which is incorporated herein by reference.

Compounds of Formula VIII would be useful for, but not limited to, the treatment of cyclooxygenase-2 mediated disorders, such as, the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, and following surgical and dental procedures.

A sub-class of phenyl heterocycle COX-2 inhibitors includes those compounds defined by Formula IX:

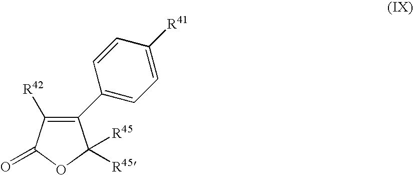

where R⁴¹ is: (a) S(O)₂CH₃; (b) S(O)₂NH₂; (c) S(O)₂NHC(O)CF₃; (d) S(O)(NH)CH₃;
(e) S(O)(NH)NH₂; (f) S(O)(NH)NHC(O)CF₃; (g) P(O)(CH₃)OH; (h) P(O)(CH₃)NH₂; (i) S(O)₂NH-alkyl; (j) S(O)₂NH-aryl; (k) S(O)₂NHC(O)-alkyl; or (l) S(O)₂NHC(O)aryl;
where R⁴² is (a) C₁₋₆ alkyl; (b) C₃, C₄, C₅, C₆, or C₇ cycloalkyl; (c) mono-, di- or tri-substituted phenyl or naphthyl wherein possible substituents include hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R⁴⁵)(R⁴⁶)—OH, —C(R⁴⁵)(R⁴⁶)—O—C₁₋₄ alkyl, and (13) —C₁₋₆ alkyl-CO₂—R⁴⁵; (d) mono-, di- or tri-substituted heteroaryl wherein the heteroaryl is a monocyclic aromatic ring of 5 atoms, the monocyclic ring having one heteroatom which is S, O, or N, and optionally 1, 2, or 3 additionally N atoms; or the heteroaryl is a monocyclic ring of 6 atoms, the monocyclic ring having one heteroatom which is N, and optionally 1, 2, or 3 additional N atoms; wherein possible substituents include hydrogen, halo (including fluoro, chloro, bromo and iodo), C₁₋₆ alkyl, C₁₋₆ alkoxy, C₁₋₆ alkylthio, CN, CF₃, N₃, —C(R⁴⁵)(R⁴⁶)—OH, and —C(R⁴⁵)(R⁴⁶)—O—C₁₋₄ alkyl;
where R⁴⁵ and R⁴⁶ are each independently: (a) hydrogen; (b) C₁₋₆ alkyl; or R⁴⁵ and R⁴⁶ together with the carbon to which they are attached form a saturated monocyclic carbon ring of 3, 4, 5, 6 or 7 atoms;

A further sub-class of phenyl heterocycle COX-2 inhibitors includes those compounds defined by Formula IX:

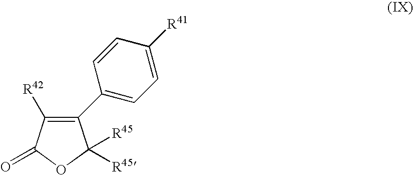

where R⁴¹ is S(O)₂CH₃, S(O)₂NH₂, S(O)NHCH₃, S(O)NHNH₂, S(O)₂NH-alkyl, S(O)₂NH-aryl, S(O)₂NHC(O)-alkyl, or S(O)₂NHC(O)aryl;
where R⁴² is C₁₋₆ alkyl; C₃, C₄, C₅, C₆, and C₇, cycloalkyl; (c) heteroaryl
(d) benzoheteroaryl (e) mono- or di-substituted phenyl wherein possible substituents include hydrogen, halo, C₁₋₆ alkoxy, C₁₋₆ alkylthio, CN, CF₃, C₁₋₆ alkyl, N₃,
—CO₂H, —CO₂—C₁₋₄ alkyl, —C(R⁴⁵)(R⁴⁶)OH, C(R⁴⁵)(R⁴⁶)—O—C₁₋₄ alkyl, or
—C₁₋₆ alkyl-CO₂—R⁴⁵;
R⁴⁵, R^{45′} and R⁴⁶ are each independently (a) hydrogen; (b) C₁₋₆ alkyl; or R⁴⁵ and R⁴⁶ together with the carbon to which they are attached form a saturated monocyclic carbon ring of 3, 4, 5, 6 or 7 atoms.

Specific examples of compounds having general formula IX include, but are not limited to:

3-(3-Fluorophenyl)-4-(4-(methylsulfonyl)phenyl)-2-(5H)-furanone; 3-(3,4-Difluorophenyl)-4-(4-(methylsulfonyl)phenyl)-2(5H)-furanone; 3-(3,4-trichlorophenyl)-4-(4-(methylsulfonyl)phenyl)-2-(5H)-furanone; 3-phenyl-4-(4-(methylsulfonyl)phenyl)-2-(5H)-furanone; 3-(3,4-Difluorophenyl)-4-(4-(methylsulfonyl)phenyl)-2(5H)-furanone; 3-phenyl-4-(4-(methylsulfonyl)phenyl)-2-(5H)-furanone; 3-(4-Fluorophenyl)-4-(4-(methylsulfonyl)phenyl)-2-(5H)-furanone; 3-(4-Fluorophenyl)-4-(4-(aminosulfonyl)phenyl)-2-(5H)-furanone; 3-(2,4-Difluorophenyl)-4-(4-(methylsulfonyl)phenyl)-2-(5H)-furanone; 3-(3,4-Difluorophenyl)-4-(4-(methylsulfonyl)phenyl)-2-(SH)-furanone; 3-(2,6-Difluorophenyl)-4-(4-(methylsulfonyl)phenyl)-2-(5H)-furanone; 3-(2,5-Difluorophenyl)-4-(4-(methylsulfonyl)phenyl)-2-(SH)-furanone; 3-(3,5-Difluorophenyl)-4-(4-(methylsulfonyl)phenyl)-2-(5H)-furanone; 3-(4-Bromophenyl)-4-(4-(methylsulfonyl)phenyl)-2-(5H)-furanone; 3-(4-Chlorophenyl)-4-(4-(methylsulfonyl)phenyl)-2-(5H)-furanone; 3-(4-Methoxyphenyl)-4-(4-(methylsulfonyl)phenyl)-2-(5H)-furanone; 3-(Phenyl)-4-(4-(methylsulfonyl)phenyl)-2-(5H)-furanone; 3-(2-Chlorophenyl)-4-(4-(methylsulfonyl)phenyl)-2-(5H)-furanone; 3-(2-Bromo-4-fluorophenyl)-4-(4-(methylsulfonyl)phenyl)-2-(5H)-furanone; 3-(2-Bromo-4-Chlorophenyl)-4-(4-(methylsulfonyl)phenyl)-2-(5H)-furanone; 3-(4-Chloro-2-fluorophenyl)-4-(4-(methylsulfonyl)phenyl)-2-(5H)-furanone; 3-(3-Bromo-4-fluorophenyl)-4-(4-(methylsulfonyl)phenyl)-2-(5H)-furanone; 3-(3-Chlorophenyl)-4-(4-(methylsulfonyl)phenyl)-2-(5H)-furanone; 3-(2-Chloro-4-fluorophenyl)-4-(4-(methylsulfonyl)phenyl)-2-(5H)-furanone; 3-(2,4-Dichlorophenyl)-4-(4-(methylsulfonyl)phenyl)-2(5H)-furanone; 3-(3,4-Dichlorophenyl)-4-(4-(methylsulfonyl)phenyl)-2(5H)-furanone; 3-(2,6-Dichlorophenyl)-4-(4-(methylsulfonyl)phenyl)-2-(5H)-furanone; 3-(3-Chloro-4-fluorophenyl)-4-(4-(methylsulfonyl)phenyl)-2-(5H)-furanone; 3-(4-Trifluoromethylphenyl)-4-(4-(methylsulfonyl)phenyl)-2-(5H)-furanone; 3-(3-Fluoro-4-methoxyphenyl)-4-(4-(methylsulfonyl)phenyl)-2-(5H)-furanone; 3-(3-Chloro-4-methoxyphenyl)-4-(4-(methylsulfonyl)phenyl)-2-(5H)-furanone; 3-(3-Fluoro-4-methoxyphenyl)-4-(4-(methylsulfonyl)phenyl)-2-(5H)-furanone; 3-(2-Fluorophenyl)-4-(4-(methylsulfonyl)phenyl)-2-(5H)-furanone; 3-(4-Methylthiophenyl)-4-(4-(methylsulfonyl)phenyl)-2(5H)-furanone; 3-(3-Fluorophenyl)-4-(4-(methylsulfonyl)phenyl)-2-(5H)-furanone; 3-(2-Chloro-6-fluorophenyl)-4-(4-(methylsulfonyl)phenyl)-2-(5H)-furanone; 3-(3-Bromo-4-methylphenyl)-4-(4-(methylsulfonyl)phenyl)-2-(5H)-furanone; 3-(4-Bromo-2-fluorophenyl)-4-(4-(methylsulfonyl)phenyl)-2-(5H)-furanone; 3-(3,4-Dibromophenyl)-4-(4-(methylsulfonyl)phenyl)-2(5H)-furanone; 3-(4-Chloro-3-fluorophenyl)-4-(4-(methylsulfonyl)phenyl)-2-(5H)-furanone; 3-(4-Bromo-3-fluorophenyl)-4-(4-(methylsulfonyl)phenyl)-2-(5H)-furanone; 3-(4-Bromo-2-chlorophenyl)-4-(4-(methylsulfonyl)phenyl)-2-(5H)-furanone; 3-(3,4-Dichlorophenyl)-4-(4-(aminosulfonyl)phenyl)-2-(5H)-furanone; 3-(3,4-Difluorophenyl)-4-(4-(aminosulfonyl)phenyl)-2-(5H)-furanone; 3-(3-Chloro-4-methoxyphenyl)-4-(4-(aminosulfonyl)phenyl)-2-(5H)-furanone; 3-(3-Bromo-4-methoxyphenyl)-4-(4(aminosulfonyl)phenyl)-2-(5H)-furanone; 3-phenyl-4-(4-(methylsulfonyl)phenyl)-2-(5H)-furanone; and pharmaceutically acceptable salts of any of the above described compounds.

A specific example of phenyl heterocycle COX-2 inhibitor includes the compound depicted below:

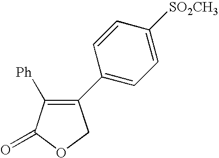

Another class of COX-2 inhibitors includes benzothiazine derivatives. In one embodiment, methods of reducing the negative cardiovascular effects associated with therapeutic administration of phenyl heterocycle COX-2 inhibitors in a subject may include administering to the subject an effective amount of a pharmaceutically acceptable formulation including a xanthophyll or other carotenoid or a synthetic analog or derivative of a xanthophyll carotenoid and one or more benzothiazine COX-2 inhibitors. Any of the carotenoids or carotenoid derivatives described herein may be used to reduce the negative cardiovascular effects associated with therapeutic administration of benzothiazine COX-2 inhibitors.

Examples of benzothiazine COX-2 inhibitors useful in treating inflammation and other COX-2 related disorders are defined by Formula X or pharmaceutically acceptable salts of the compounds defined by Formula X:

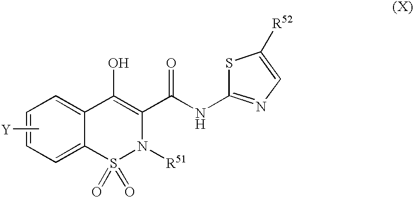

where R⁵¹ is hydrogen, methyl or ethyl;
where R⁵² is methyl, ethyl or n-propyl; and
where Y is hydrogen, methyl, methoxy, fluorine or chlorine.

A description of the synthesis and use of benzothiazine COX-2 inhibitors can be found in U.S. Pat. No. 4,233,299, which is incorporated herein by reference.

Compounds of Formula X would be useful for, but not limited to, the treatment of cyclooxygenase-2 mediated disorders, such as, the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, and following surgical and dental procedures.

A specific example of a benzothiazine COX-2 inhibitor includes the compound depicted below:

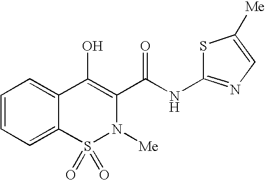

Another class of COX-2 inhibitors includes substituted pyridines. In one embodiment, methods of reducing the negative cardiovascular effects associated with therapeutic administration of substituted pyridine COX-2 inhibitors in a subject may include administering to the subject an effective amount of a pharmaceutically acceptable formulation including a xanthophyll or other carotenoid or a synthetic analog or derivative of a xanthophyll carotenoid and one or more substituted pyridine COX-2 inhibitors. Any of the carotenoids or carotenoid derivatives described herein may be used to reduce the negative cardiovascular effects associated with therapeutic administration of substituted pyridine COX-2 inhibitors.

Examples of substituted pyridine COX-2 inhibitors useful in treating inflammation and other COX-2 related disorders are defined by Formula XI or pharmaceutically acceptable salts of the compounds defined by Formula XI:

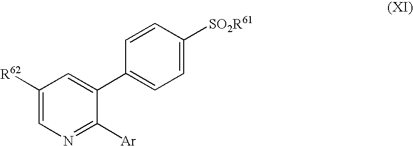

where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where Ar is a mono-, di-, or trisubstituted phenyl or pyridinyl (or the N-oxide thereof), wherein substituents include hydrogen, halogen, C₁₋₆ alkoxy, C₁₋₆alkylthio, CN, C₁₋₆alkyl, C₁₋₆-fluoroalkyl, N₃, —CO₂R⁶³, hydroxy, —C(R⁶⁴)(R⁶⁵)—OH, —C₁₋₆ alkyl-CO₂—R⁶⁶, or C₁₋₆-fluoroalkoxy; where R⁶² is: (a) halo; (b) C₁₋₆alkoxy; (c) C₁₋₆alkylthio; (d) CN; (e) C₁₋₆alkyl;
(f) C₁₋₆ fluoroalkyl; (g) N₃; (h) —CO₂R⁶⁷; (i) hydroxy; (j) —C(R⁶⁸)(R⁶⁹)—OH;
(k) —C₁₋₆ alkyl-CO₂—R⁷⁰; (l) C₁₋₆-fluoroalkoxy; (m) NO₂; (n) NR⁵³R⁵⁴; and

### (o) NHCOR⁵⁵;

where R⁶³, R⁶⁴, R⁶⁵, R⁶⁶, R⁶⁷, R⁶⁸, R⁶⁹, R⁷⁰, R⁵³, R⁵⁴, R⁵⁵, are each independently hydrogen or C₁₋₆alkyl, or R⁶⁴ and R⁶⁵, R⁶⁸ and R⁶⁹ or R⁵³ and R⁵⁴ together with the atom to which they are attached form a saturated monocyclic ring of 3, 4, 5, 6 or 7 atoms.

A description of the synthesis and use of substituted pyridine COX-2 inhibitors can be found in U.S. Pat. No. 5,861,419, which is incorporated herein by reference.

Compounds of Formula XI would be useful for, but not limited to, the treatment of cyclooxygenase-2 mediated disorders, such as, the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, and following surgical and dental procedures.

A further sub-class of substituted pyridine COX-2 inhibitors includes those compounds defined by Formula XII:

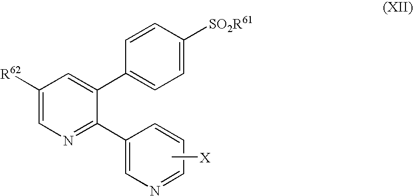

where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where R⁶² is: (a) halo; (b) C₁₋₃ alkoxy; (c) C₁₋₃ alkylthio; (d) C₁₋₃ alkyl; (e) N₃; (f) —CO₂H;
(g) hydroxy; (h) C₁₋₃ fluoroalkoxy; (i) NO₂; (j) NR⁵³R⁵⁴ and (k) NHCOR⁵⁵;
where X is methyl, ethyl, n-propyl, i-propyl or cyclopropyl.

A further sub-class of substituted pyridine COX-2 inhibitors includes those compounds defined by Formula XIII:

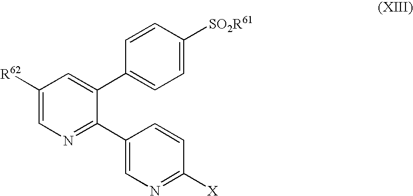

where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where R⁶² is chloro or methyl;
and where there may be one, two or three X groups, where each X group is independently: hydrogen, halogen, C₁₋₄ alkoxy, C₁₋₄ alkylthio, CN, C₁₋₄ alkyl, or CF₃.

Specific examples of compounds having general formula XI, XII and XIII include, but are not limited to:

3-(4-Methylsulfonyl)phenyl-2-(3-pyridinyl)-5-trifluoromethylpyridine; 5-Methyl-3-(4-methylsulfonyl)phenyl-2-(3-pyridinyl)pyridine; 5-Chloro-3-(4-methylsulfonyl)phenyl-2-(2-pyridinyl)pyridine; 5-Chloro-3-(4-methylsulfonyl)phenyl-2-(3-pyridinyl)pyridine; 5-Chloro-3-(4-methylsulfonyl)phenyl-2-(4-pyridinyl)pyridine; 5-Chloro-3-(4-methylsulfonyl)phenyl-2-(2-methyl-5-pyridinyl)pyridine; 5-Chloro-3-(4-methylsulfonyl)phenyl-2-(3-pyridyl)pyridine hydromethanesulfonate; 5-Chloro-3-(4-methylsulfonyl)phenyl-2-(3-pyridyl)pyridine hydrochloride; 5-Chloro-3-(4-methylsulfonyl)phenyl-2-(2-methyl-5-pyridinyl)pyridine Hydrochloride; 5-Chloro-3-(4-methylsulfonyl)phenyl-2-(2-ethyl-5-pyridinyl)pyridine; and 5-Chloro-3-(4-methylsulfonyl)phenyl-2-(2-ethyl-5-pyridinyl)pyridine hydromethanesulfonate.

A specific example of a benzothiazine COX-2 inhibitor includes the compound depicted below:

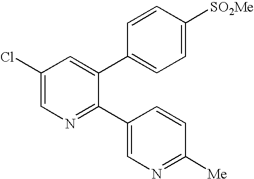

Another class of COX-2 inhibitors includes substituted arylaminophenylacetic acids. In one embodiment, methods of reducing the negative cardiovascular effects associated with therapeutic administration of substituted arylaminophenylacetic acid COX-2 inhibitors in a subject may include administering to the subject an effective amount of a pharmaceutically acceptable formulation including a xanthophyll or other carotenoid or a synthetic analog or derivative of a xanthophyll carotenoid and one or more arylaminophenylacetic acid COX-2 inhibitors. Any of the carotenoids or carotenoid derivatives described herein may be used to reduce the negative cardiovascular effects associated with therapeutic administration of substituted arylaminophenylacetic acid COX-2 inhibitors.

Examples of substituted arylaminophenylacetic acid COX-2 inhibitors useful in treating inflammation and other COX-2 related disorders are defined by Formula XIV or pharmaceutically acceptable salts of the compounds defined by Formula XIV:

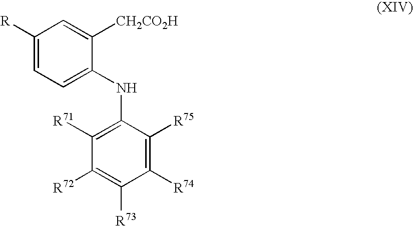

R is methyl or ethyl; R⁷¹ is chloro or fluoro; R⁷² is hydrogen or fluoro; R⁷³ is hydrogen, fluoro, chloro, methyl, ethyl, methoxy, ethoxy or hydroxy; R⁷⁴ is hydrogen or fluoro; and R⁷⁵ is chloro, fluoro, trifluoromethyl or methyl; pharmaceutically acceptable salts thereof; and pharmaceutically acceptable prodrug esters thereof. Pharmaceutically acceptable prodrug esters are ester derivatives which are convertible by solvolysis or under physiological conditions to the free carboxylic acids of formula XIV. Such esters are e.g. lower alkyl esters (such as the methyl or ethyl ester), carboxy-lower alkyl esters such as the carboxymethyl ester, nitrooxy-lower alkyl esters (such as the 4-nitrooxybutyl ester), and the like. In one embodiments, ester prodrugs include 5-alkyl substituted 2-arylaminophenylacetoxyacetic acids. Pharmaceutically acceptable salts represent metal salts, such as alkaline metal salts, e.g. sodium, potassium, magnesium or calcium salts, as well as ammonium salts, which are formed e.g. with ammonia and mono- or di-alkylamines, such as diethylammonium salts, and with amino acids, such as arginine and histidine salts. A description of the synthesis and use of substituted arylaminophenylacetic acid COX-2 inhibitors can be found in U.S. Pat. No. 6,310,099, which is incorporated herein by reference.

Compounds of Formula XIV would be useful for, but not limited to, the treatment of cyclooxygenase-2 mediated disorders, such as, the relief of pain, fever and inflammation of a variety of conditions including rheumatic fever, symptoms associated with influenza or other viral infections, common cold, low back and neck pain, dysmenorrhea, headache, toothache, sprains and strains, myositis, neuralgia, synovitis, arthritis, including rheumatoid arthritis degenerative joint diseases (osteoarthritis), gout and ankylosing spondylitis, bursitis, burns, injuries, and following surgical and dental procedures.

Specific examples of compounds having general formula XIV include, but are not limited to:

Compounds of formula XIV wherein R is methyl or ethyl; R⁷¹ is chloro or fluoro; R⁷² is hydrogen; R⁷³ is hydrogen, fluoro, chloro, methyl or hydroxy; R⁷⁴ is hydrogen; and R⁷⁵ is chloro, fluoro or methyl; pharmaceutically acceptable salts thereof; and pharmaceutically acceptable prodrug esters thereof.

Compounds of formula XIV wherein R is methyl or ethyl; R⁷¹ is fluoro; R⁷² is hydrogen; R⁷³ is hydrogen, fluoro or hydroxy; R⁷⁴ is hydrogen; and R⁷⁵ is chloro; pharmaceutically acceptable salts thereof; and pharmaceutically acceptable prodrug esters thereof.

Compounds of formula XIV wherein R is ethyl or methyl; R⁷¹ is fluoro; R⁷² is hydrogen or fluoro; R⁷³ is hydrogen, fluoro, ethoxy or hydroxy; R⁷⁴ is hydrogen or fluoro; and R⁷⁵ is chloro, fluoro or methyl; pharmaceutically acceptable salts thereof; and pharmaceutically acceptable prodrug esters thereof.

Compounds of formula XIV wherein R is methyl or ethyl; R⁷¹ is fluoro; R⁷²-R⁷⁴ are hydrogen or fluoro; and R⁷⁵ is chloro or fluoro; pharmaceutically acceptable salts thereof; and pharmaceutically acceptable prodrug esters thereof.

Compounds of formula XIV wherein R is methyl or ethyl; R⁷¹ is fluoro; R⁷² is fluoro; R⁷³ is hydrogen, ethoxy or hydroxy; R⁴ is fluoro; and R⁷⁵ is fluoro; pharmaceutically acceptable salts thereof; and pharmaceutically acceptable prodrug esters thereof.

Compounds of formula XIV wherein R is methyl; R⁷¹ is fluoro; R⁷² is hydrogen; R⁷³ is hydrogen or fluoro; R⁷⁴ is hydrogen; and R⁷⁵ is chloro; pharmaceutically acceptable salts thereof; and pharmaceutically acceptable prodrug esters thereof.

Compounds of formula XIV wherein R is methyl; R⁷¹ is fluoro; R⁷² is hydrogen; R⁷³ is hydrogen; R⁷⁴ is hydrogen; and R⁷⁵ is chloro; pharmaceutically acceptable salts thereof; and pharmaceutically acceptable prodrug esters thereof;

Compounds of formula XIV wherein R is methyl; R⁷¹ is fluoro; R⁷² is hydrogen; R⁷³ is fluoro; R⁷⁴ is hydrogen; and R⁷⁵ is chloro; pharmaceutically acceptable salts thereof; and pharmaceutically acceptable prodrug esters thereof;

Compounds of formula XIV wherein R is ethyl; R⁷¹ is fluoro; R⁷² is fluoro; R⁷³ is hydrogen; R⁷⁴ is fluoro; and R⁷⁵ is fluoro; pharmaceutically acceptable salts thereof; and pharmaceutically acceptable prodrug esters thereof; and

Compounds of formula XIV wherein R is ethyl; R⁷¹ is chloro; R⁷² is hydrogen; R⁷³ is chloro; R⁷⁴ is hydrogen; and R⁷⁵ is methyl; pharmaceutically acceptable salts thereof; and pharmaceutically acceptable prodrug esters thereof.

A specific example of an arylaminophenylacetic acid COX-2 inhibitor includes the compound depicted below:

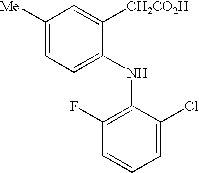

Several of the above mentioned COX-2 selective inhibitors have been approved for human use or are in advanced stage of development; accordingly, one subset of COX-2 selective inhibitors of the present invention include rofecoxib (Vioxx®), celecoxib (Celebrex®), valdecoxib (Bextra®), meloxicam (Mobic®), lumiracoxib (Prexige®), parecoxib (Dynastat®), and etoricoxib (Arcoxia®). In one embodiment, methods of reducing the negative cardiovascular effects associated with the specific above-listed COX-2 inhibitors in a subject may include administering to the subject an effective amount of a pharmaceutically acceptable formulation including a xanthophyll or other carotenoid or a synthetic analog or derivative thereof. Any of the carotenoids or carotenoid derivatives described herein may be used to reduce the negative cardiovascular effects associated with therapeutic administration of the specific above-listed COX-2 inhibitors.

### Carotenoids and the Preparation and Use Thereof

In some embodiments, a composition may include one or more carotenoids and one or more COX-2 inhibitors. Carotenoids may include carotenes and xanthophyll carotenoids. In some embodiments, carotenoids that may be combined with one or more COX-2 inhibitors (e.g., the COX-2 inhibitors described above) include carotenoids having the general structure:

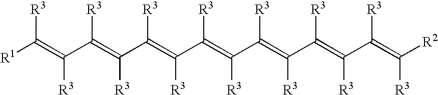

where each R³ is independently hydrogen or methyl, and where each R¹ and R² are independently:

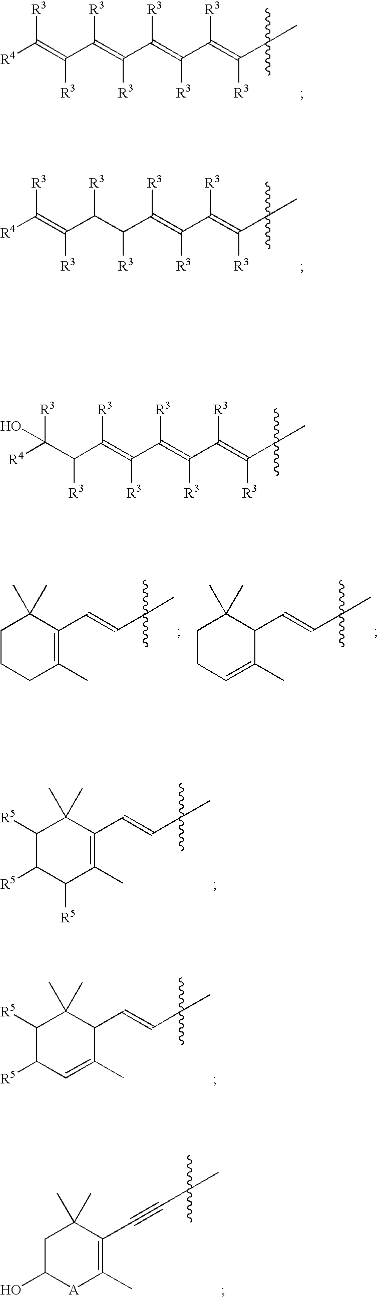

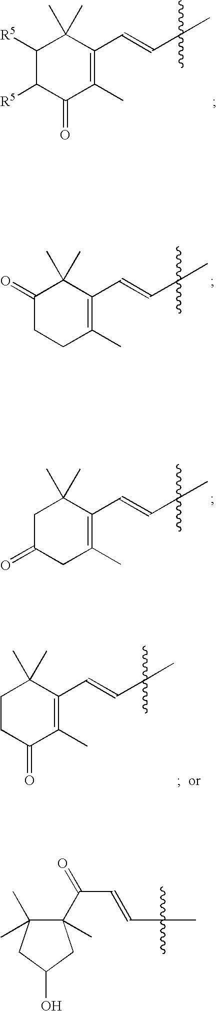

where R⁴ is hydrogen, methyl, or —CH₂OH; and where each R⁵ is independently hydrogen or —OH. Sources of some of these carotenoids can be found, for example, in the reference “Key to Cartenoids”, Otto Straub, 2^{nd} Ed., Birkhauser Verlag, Boston, 1987, which is incorporated herein by reference.

In some embodiments, carotenoids that may be combined with one or more COX-2 inhibitors include carotenoids having the general structure:

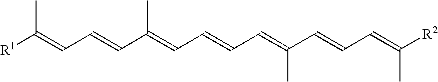

where each R¹ and R² are independently:

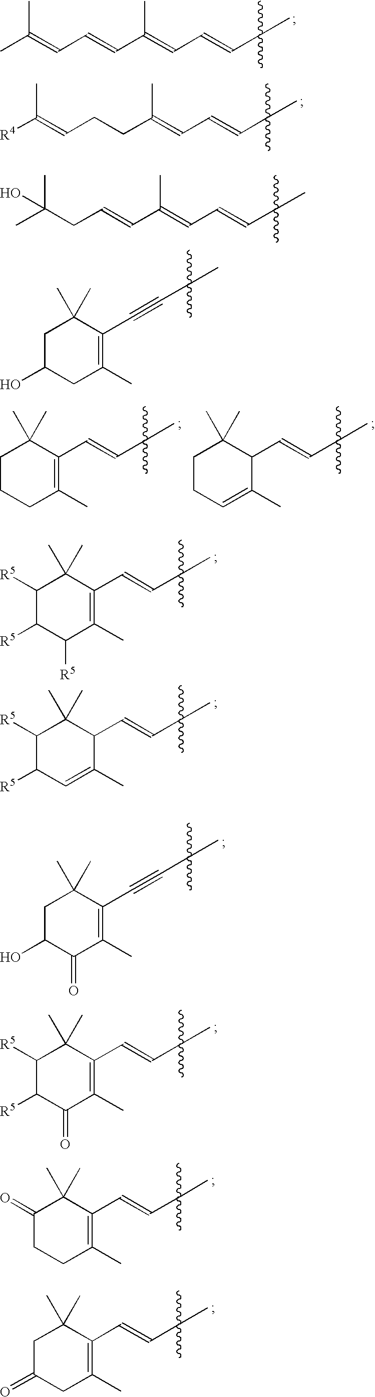

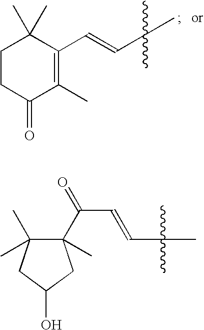

where R⁴ is hydrogen, methyl, or —CH₂OH; and where each R⁵ is independently hydrogen or —OH.

In some embodiments, carotenoids that may be combined with one or more COX-2 inhibitors include xanthophyll carotenoids having the general structure:

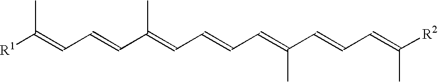

where each R¹ and R² are independently:

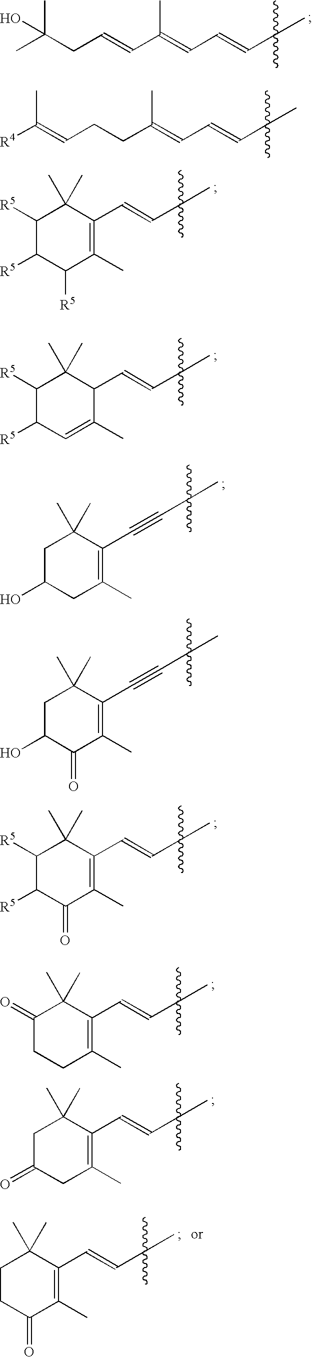

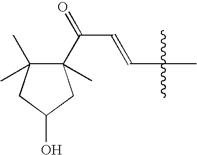

where R⁴ is —CH₂—OH; and where each R⁵ is independently hydrogen or —OH.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate derivatives, succinate derivatives, co-antioxidant derivatives (e.g., Vitamin C, Vitamin C analogs, Vitamin C derivatives, Vitamin E, Vitamin E analogs, Vitamin E derivatives, flavonoids, flavonoid analogs, or flavonoid derivatives), or combinations thereof derivatives or analogs of carotenoids. Flavonoids may include, for example, quercetin, xanthohumol, isoxanthohumol, or genistein. Vitamin E may generally be divided into two categories including tocopherols having a general structure

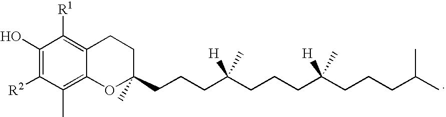

Alpha-tocopherol is used to designate when R¹═R²═CH₃. Beta-tocopherol is used to designate when R¹═CH₃ and R²═H. Gamma-tocopherol is used to designate when R¹═H and R²═CH₃. Delta-tocopherol is used to designate when R¹═R²═H.

The second category of Vitamin E may include tocotrienols having a general structure

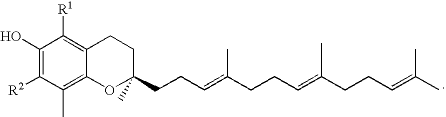

Alpha-tocotrienol is used to designate when R¹═R²═CH₃. Beta-tocotrienol is used to designate when R¹═CH₃ and R²═H. Gamma-tocotrienol is used to designate when R¹═H and R²═CH₃. Delta-tocotrienol is used to designate when R¹═R²═H.

Quercetin, a flavonoid, has the structure

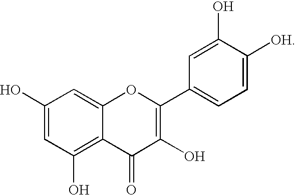

In some embodiments, one or more co-antioxidants may be coupled to a carotenoid or carotenoid derivative or analog. Derivatives of one or more carotenoid analogues may be formed by coupling one or more free hydroxy groups of the co-antioxidant to a portion of the carotenoid.

Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

In some embodiments, carotenoid analogs or derivatives may have increased water solubility and/or water dispersibility relative to some or all known naturally occurring carotenoids. Contradictory to previous research, improved results are obtained with derivatized carotenoids relative to the base carotenoid, wherein the base carotenoid is derivatized with substituents including hydrophilic substituents and/or co-antioxidants.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

In some embodiments, a chemical compound including a carotenoid derivative or analog may have the general structure:

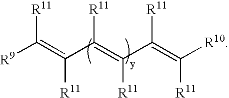

Each R¹¹ may be independently hydrogen or methyl. R⁹ and R¹⁰ may be independently H, an acyclic alkene with one or more substituents, or a cyclic ring including one or more substituents. y may be 5 to 12. In some embodiments, y may be 3 to 15. In certain embodiments, the maximum value of y may only be limited by the ultimate size of the chemical compound, particularly as it relates to the size of the chemical compound and the potential interference with the chemical compound's biological availability as discussed herein. In some embodiments, substituents may be at least partially hydrophilic. These carotenoid derivatives may be included in a pharmaceutical composition.

In some embodiments, a method of inhibiting or reducing the at least some of the side effects associated with therapeutic administration of COX-2 selective inhibitors may include administering to the subject an effective amount of a pharmaceutically acceptable formulation including one or more synthetic analogs or derivatives of a carotenoid. The synthetic analog or derivative of the carotenoid may have the structure

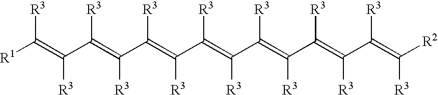

where each R³ is independently hydrogen or methyl, and where each R¹ and R² are independently:

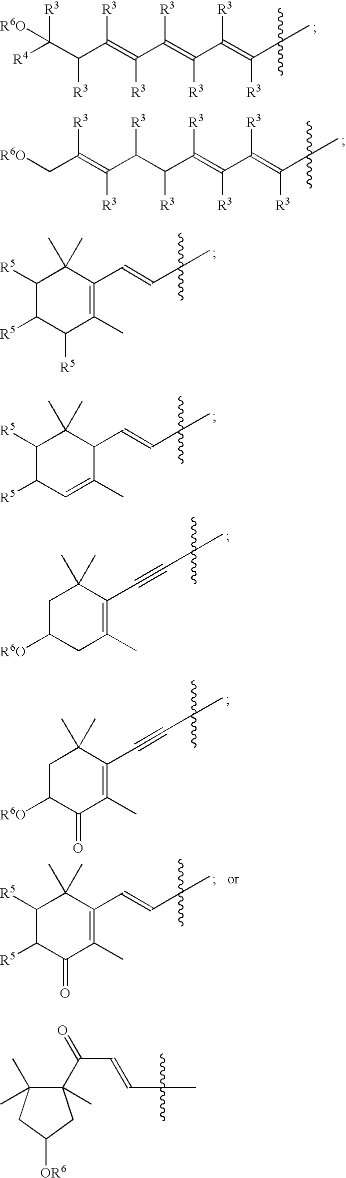

where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a con-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with therapeutic administration of COX-2 selective inhibitors

Each co-antioxidant may be independently Vitamin C, Vitamin C analogs, Vitamin C derivatives, Vitamin E, Vitamin E analogs, Vitamin E derivatives, flavonoids, flavonoid derivatives, or flavonoid analogs. Flavonoids include, but are not limited to, quercetin, xanthohumol, isoxanthohumol, or genistein. Selection of the co-antioxidant should not be seen as limiting for the therapeutic application of the current invention.

In some embodiments, a method of inhibiting or reducing the at least some of the side effects associated with therapeutic administration of COX-2 selective inhibitors may include administering to the subject an effective amount of a pharmaceutically acceptable formulation including one or more synthetic analogs or derivatives of a carotenoid. The synthetic analog or derivative of the carotenoid may have the structure

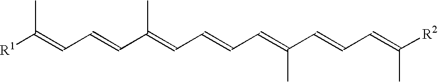

where each R¹ and R² are independently:

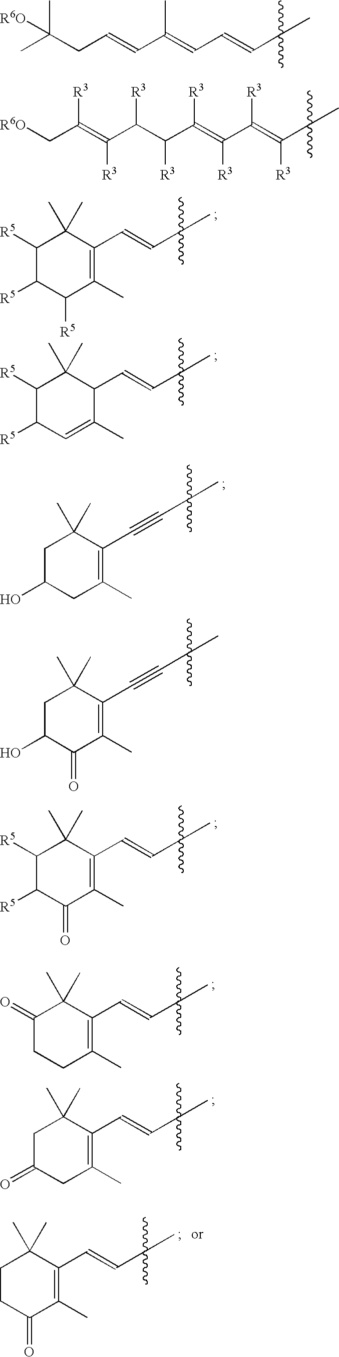

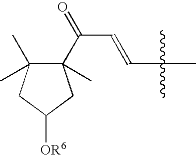

where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9. Pharmaceutically acceptable salts of any of the above listed carotenoid derivatives may also be used to ameliorate at least some of the side effects associated with therapeutic administration of COX-2 selective inhibitors

When R⁶ is an amino acid derivative or a peptide, coupling of the amino acid or the peptide is accomplished through an ester linkage. The ester linkage may be formed between a free hydroxyl of the xanthophyll carotene and the carboxylic acid of the amino acid or peptide. When R⁹ is an amino acid derivative or a peptide, coupling of the amino acid or the peptide is accomplished through an amide linkage. The amide linkage may be formed between the terminal carboxylic acid group of the linker attached to the xanthophyll carotene and the amine of the amino acid or peptide.

When R⁶ is a sugar, R⁶ includes, but is not limited to the following side chains:

—CH₂—(CHOH)ₙ—CO₂H;

—CH₂—(CHOH)ₙ—CHO;

—CH₂—(CHOH)ₙ—CH₂OH;

—CH₂—(CHOH)ₙ—C(O)—CH₂OH;

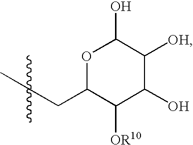

where R¹⁰ is hydrogen or

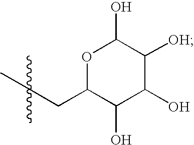

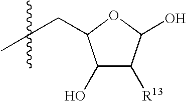

where R¹³ is hydrogen or —OH.

When R⁶ is a nucleoside, R⁶ may have the structure:

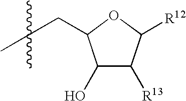

where R¹² is a purine or pyrimidine base, and R¹³ is hydrogen or —OH.

In some embodiments, the carotenoid analog or derivative may have the structures

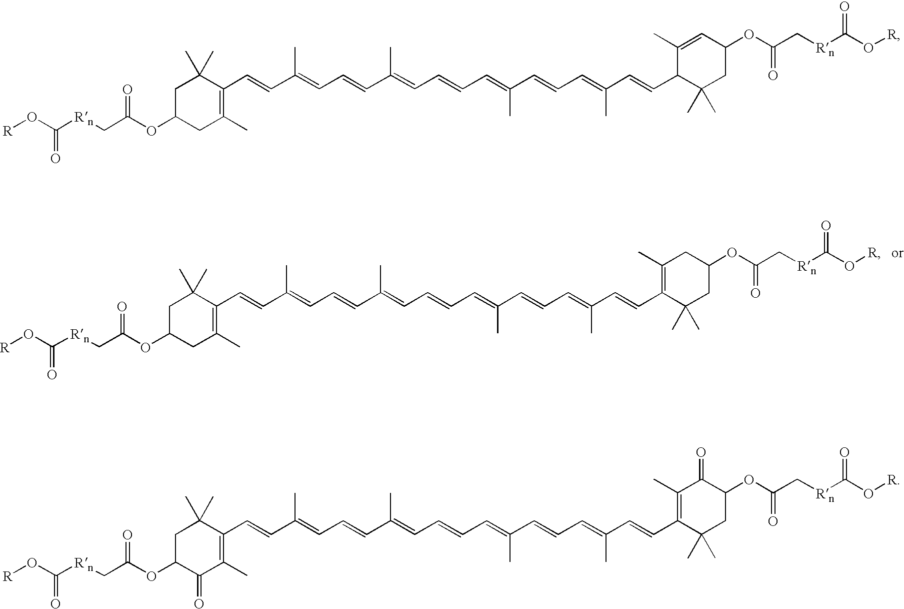

Each R may be independently H, alkyl, aryl, benzyl, Group IA metal, or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein.

In some embodiments, the carotenoid analog or derivative may have the structures

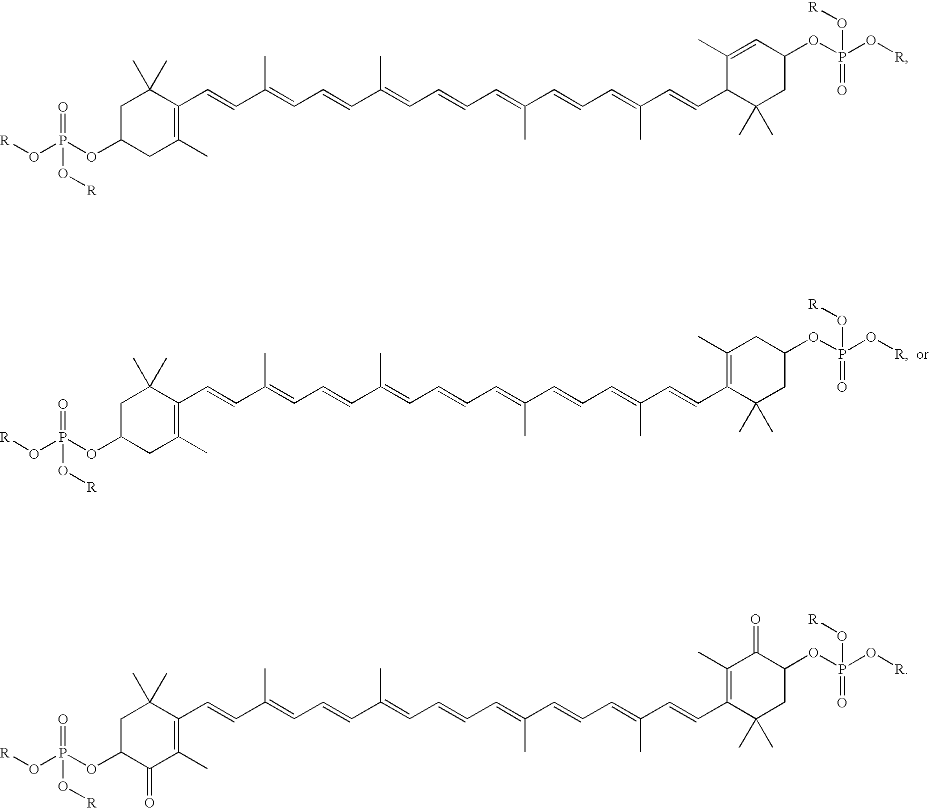

Each R may be independently H, alkyl, aryl, benzyl, Group IA metal (e.g., sodium), or a co-antioxidant. Each co-antioxidant may be independently Vitamin C, Vitamin C analogs, Vitamin C derivatives, Vitamin E, Vitamin E analogs, Vitamin E derivatives, flavonoids, flavonoid analogs, or flavonoid derivatives. Flavonoids may include, for example, quercetin, xanthohumol, isoxanthohumol, or genistein. When R includes Vitamin C, Vitamin C analogs, or Vitamin C derivatives, some embodiments may include carotenoid analogs or derivatives having the structure

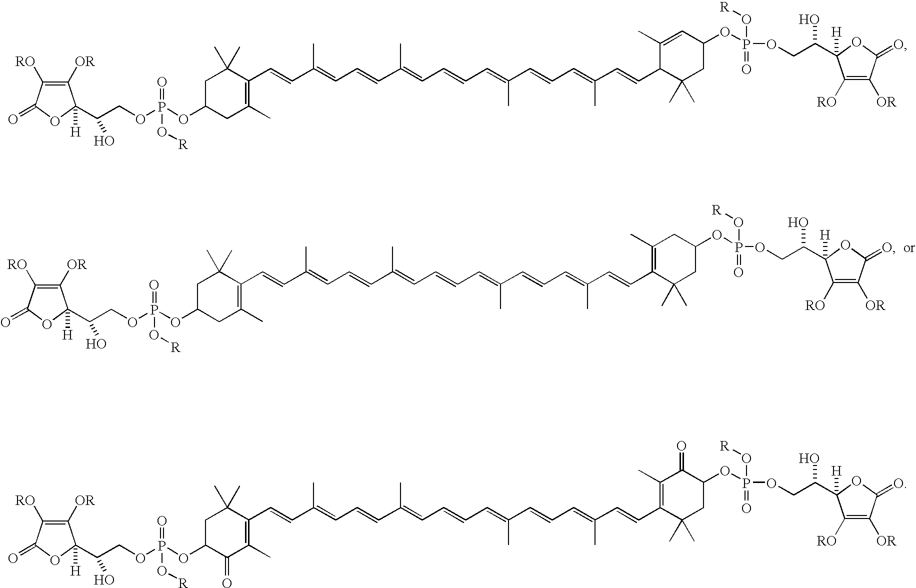

Each R may be independently H, alkyl, aryl, benzyl, or Group IA metal.

In some embodiments, the carotenoid derivative may have the structure:

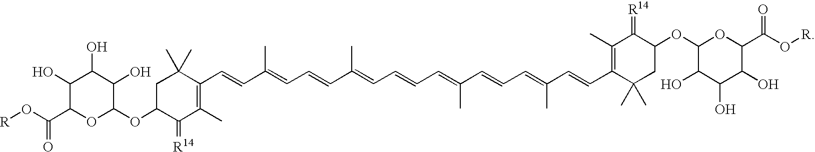

Each R¹⁴ may be independently 0 or H₂. Each R may be independently H, alkyl, benzyl, Group IA metal, co-antioxidant, or aryl.

Specific examples of carotenoid derivatives include, but are not limited to, the following compounds:

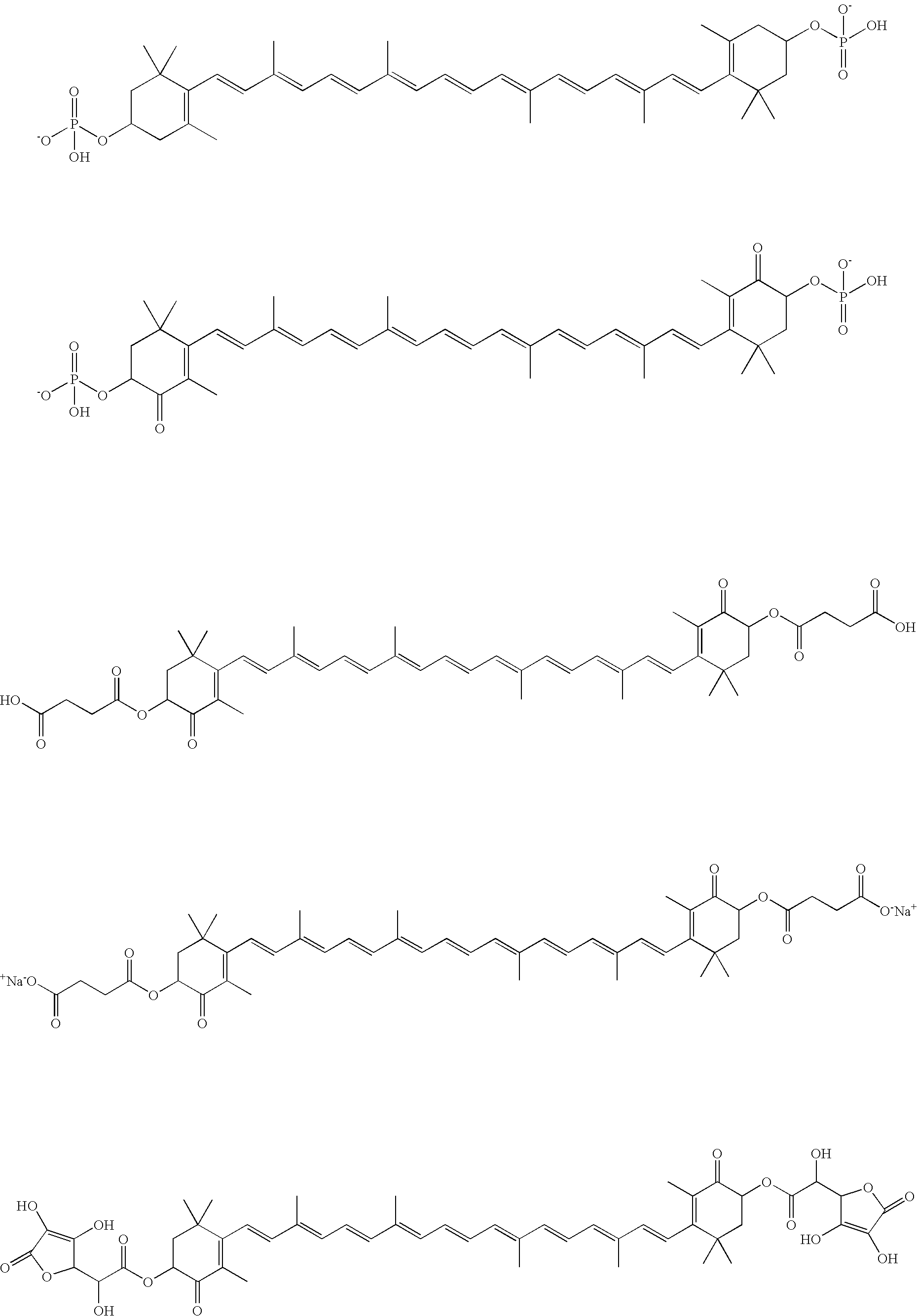

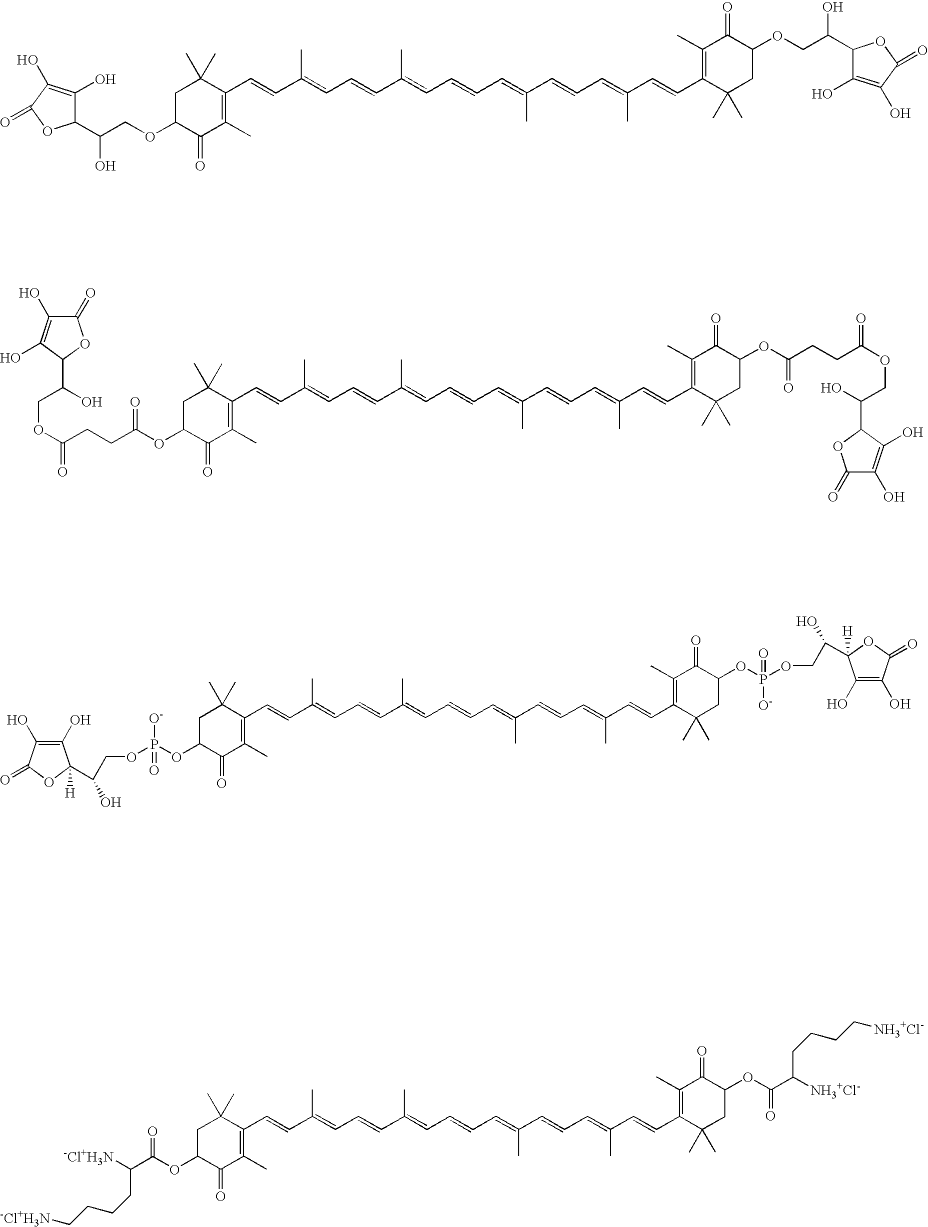

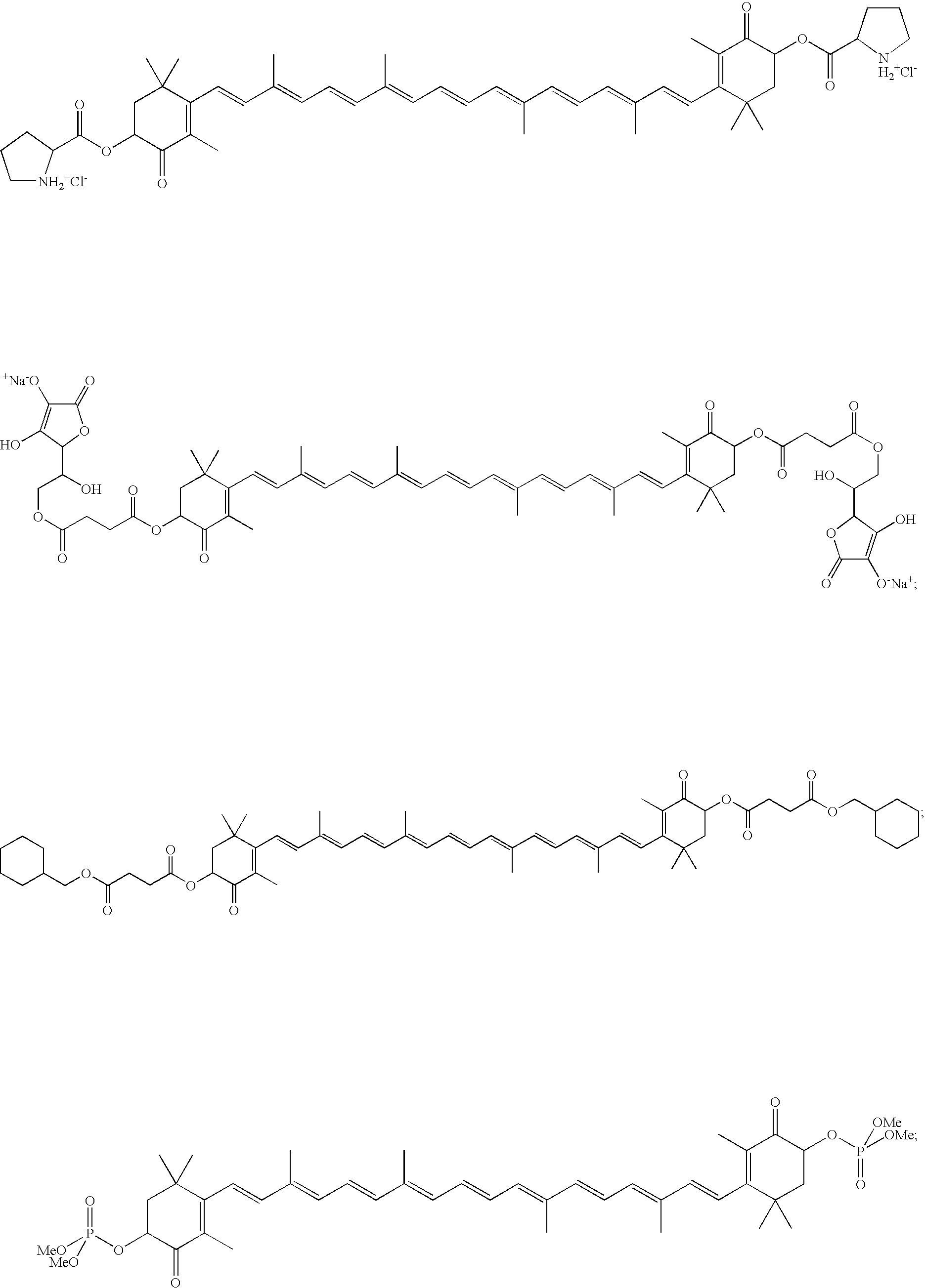

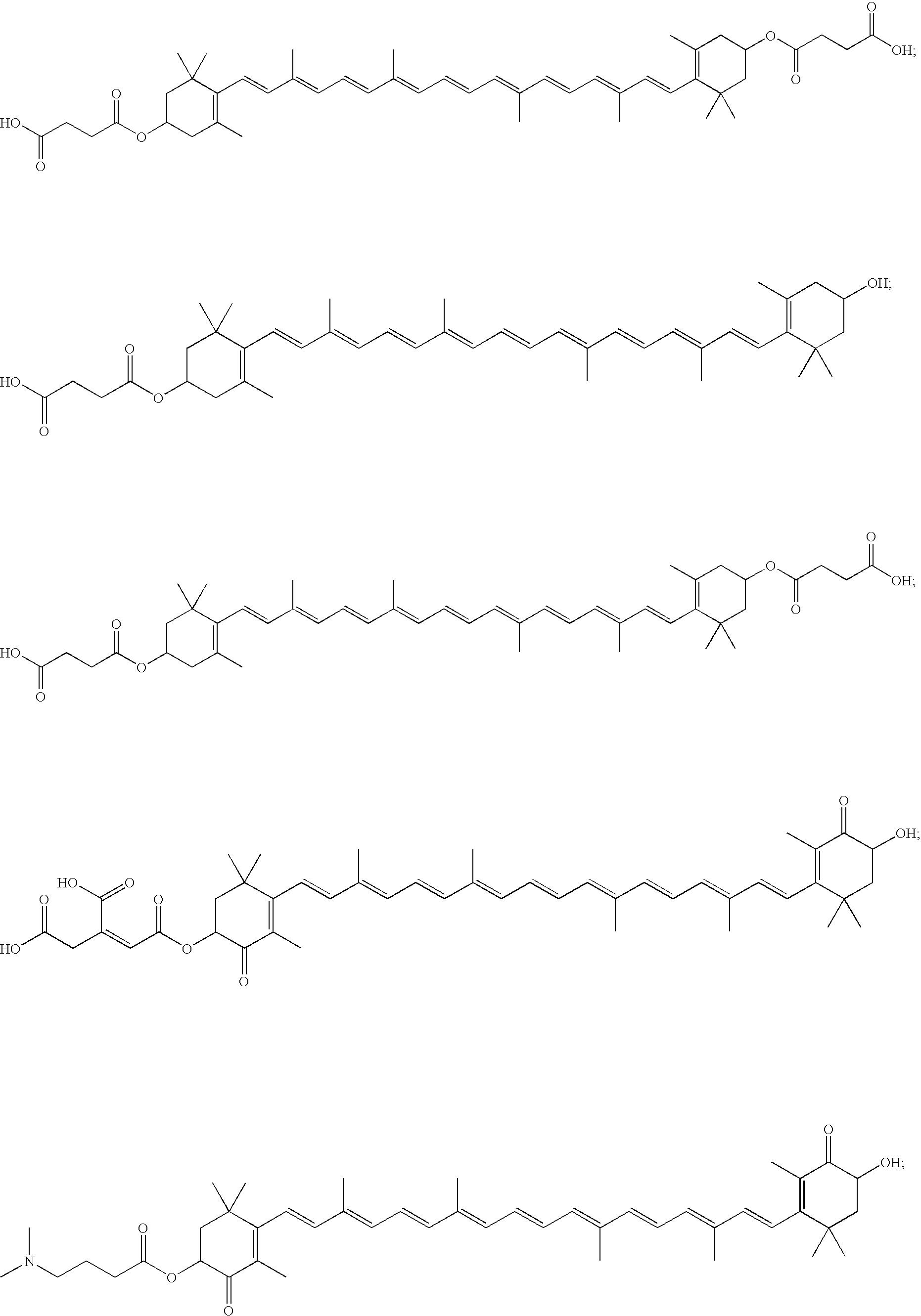

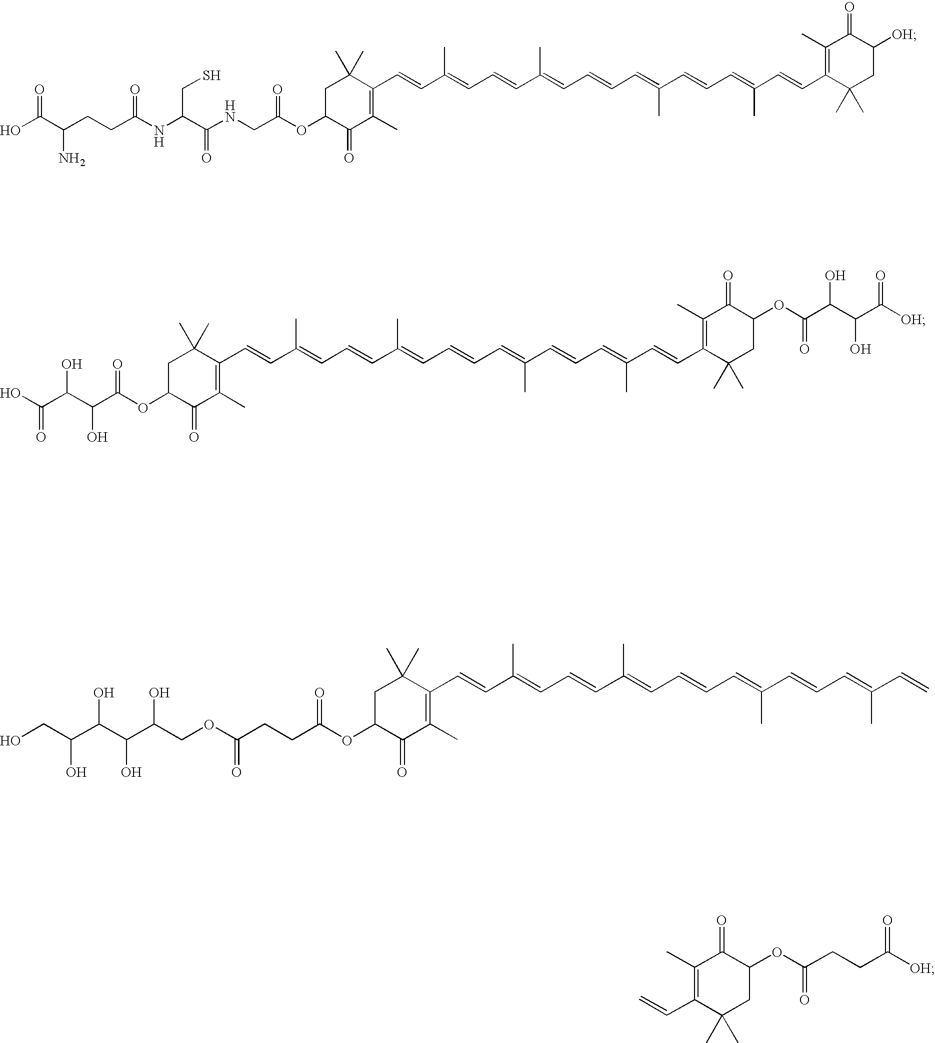

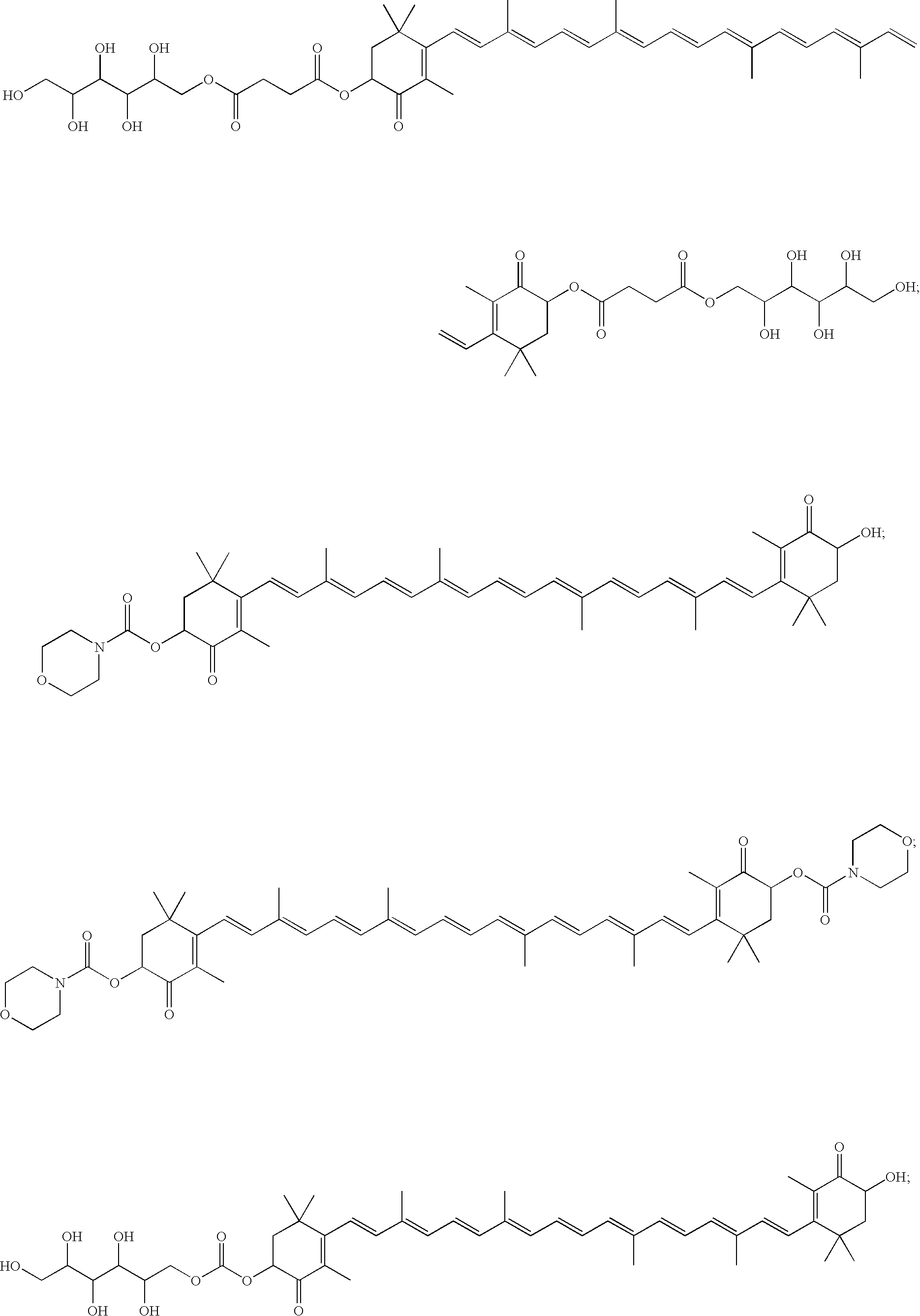

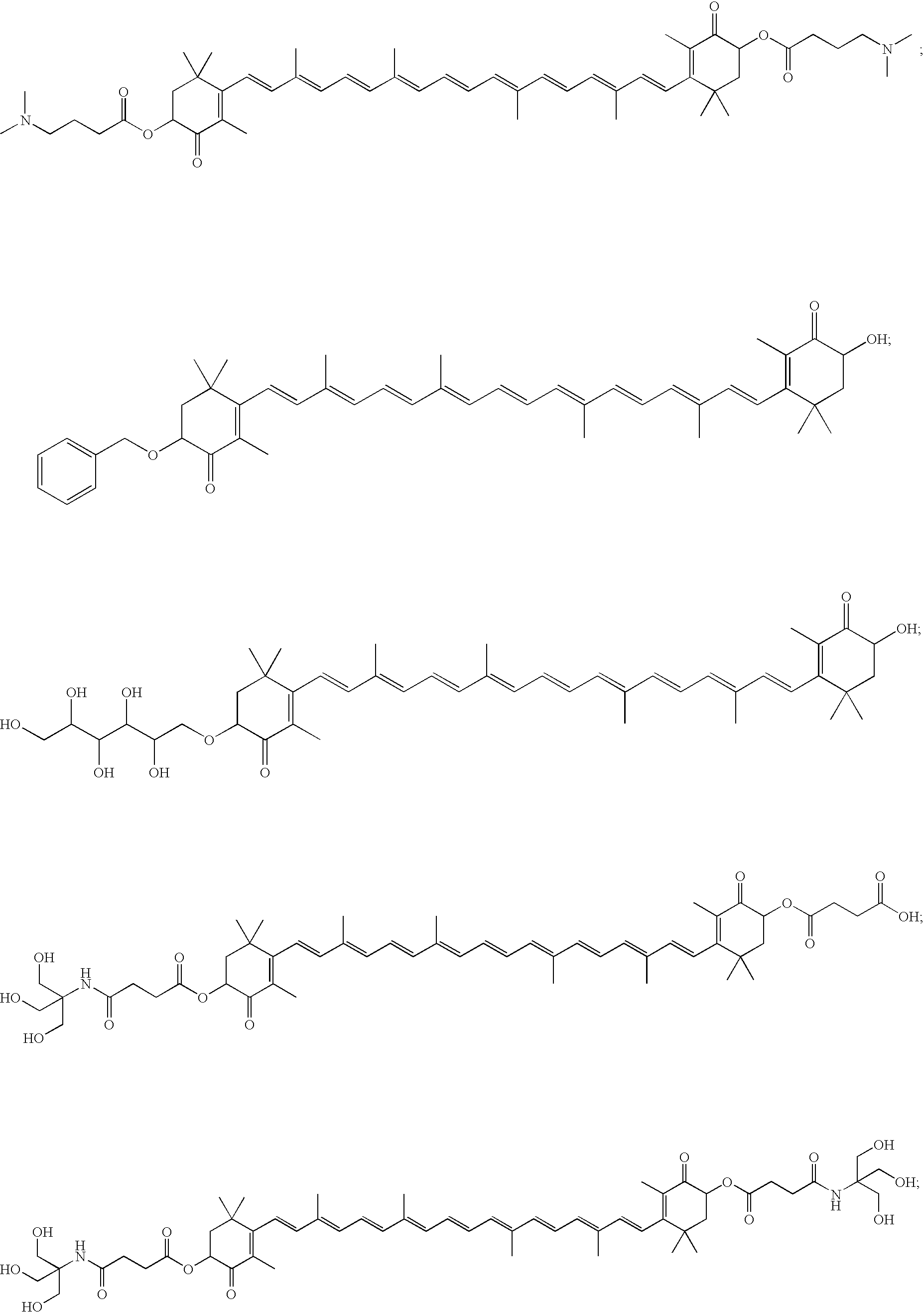

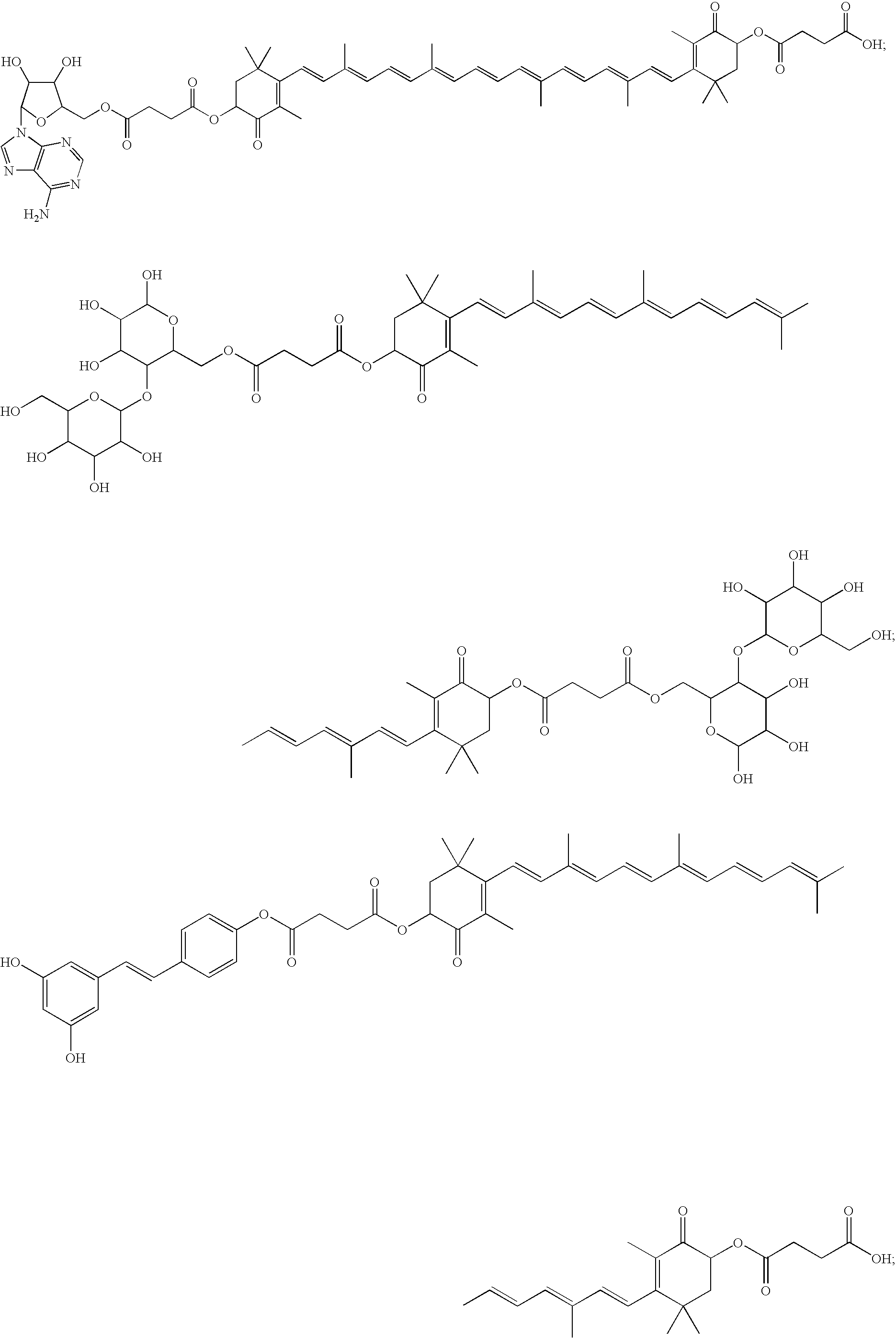

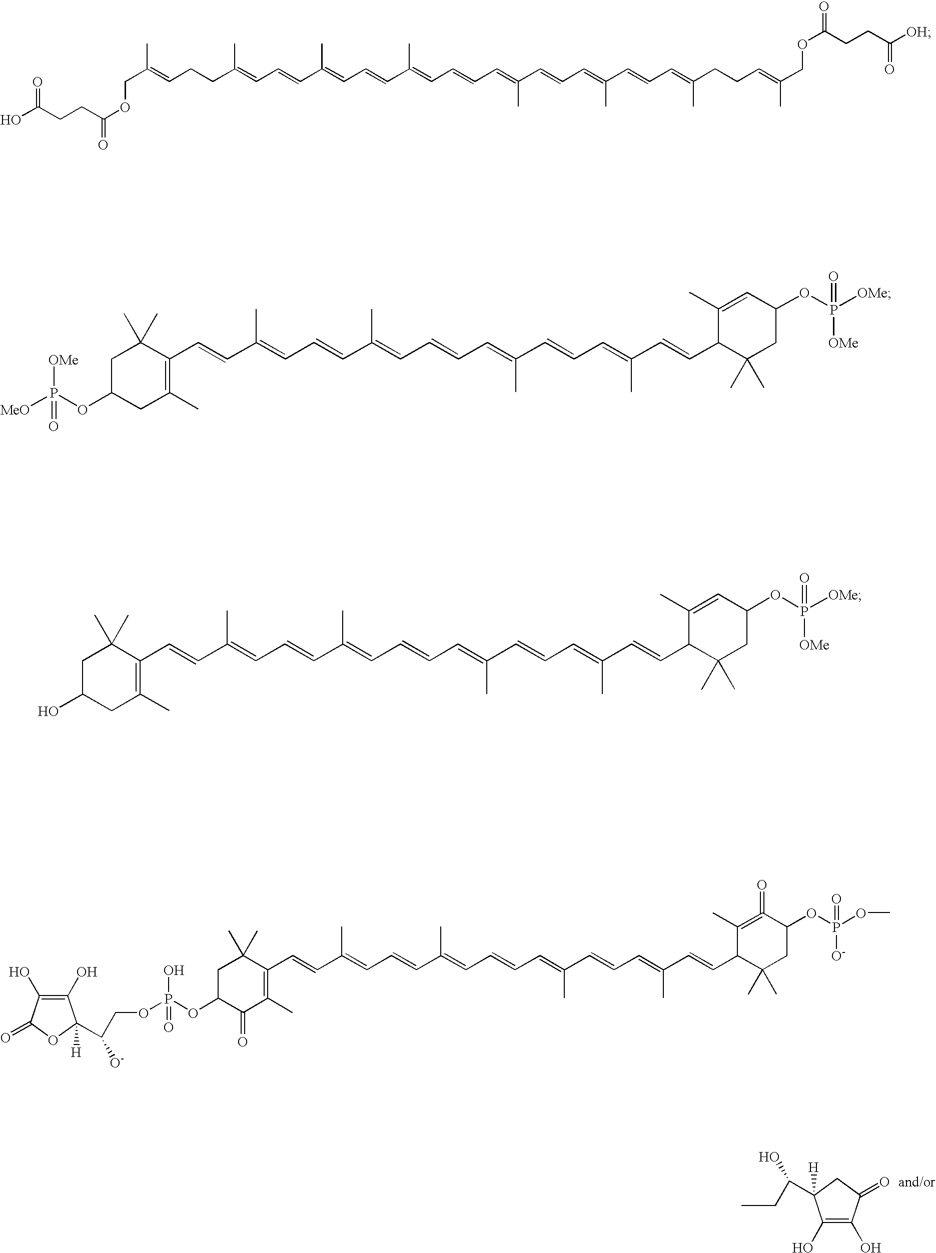

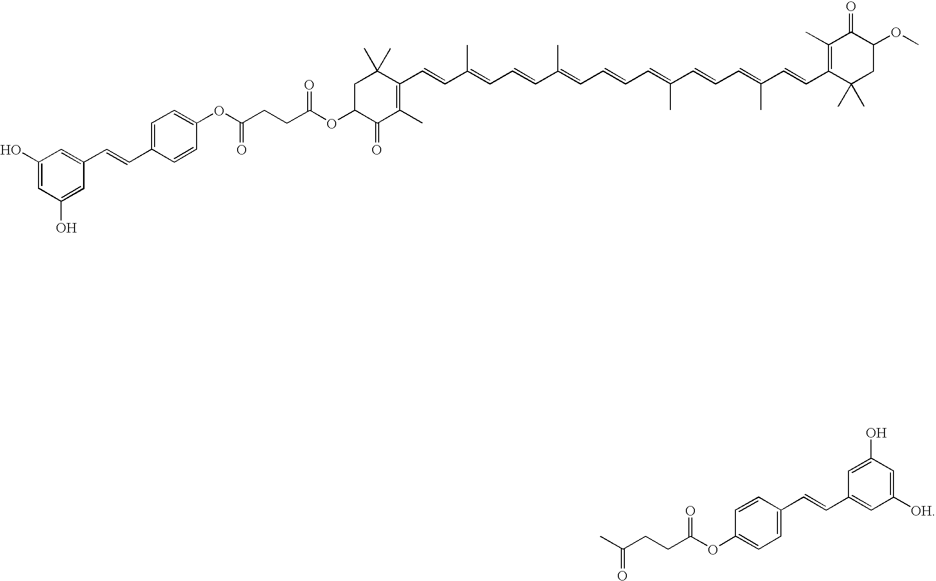

Further details regarding the synthesis of carotenoid derivatives and analogs is illustrated in U.S. patent application Ser. No. 10/793,671 filed on Mar. 4, 2004, entitled “CAROTENOID ETHER ANALOGS OR DERIVATIVES FOR THE INHIBITION AND AMELIORATION OF DISEASE” by Lockwood et al. published on Jan. 13, 2005, as Publication No. US-2005-0009758 and PCT International Application Number PCT/US2003/023706 filed on Jul. 29, 2003, entitled “STRUCTURAL CAROTENOID ANALOGS FOR THE INHIBITION AND AMELIORATION OF DISEASE” by Lockwood et al. (International Publication Number WO 2004/011423 A2, published on Feb. 5, 2004) both of which are incorporated by reference as though fully set forth herein.

Water-soluble carotenoid analogs or derivatives may have a water solubility of greater than about 1 mg/mL in some embodiments. In certain embodiments, water-soluble carotenoid analogs or derivatives may have a water solubility of greater than about 5 mg/mL. In certain embodiments, water-soluble carotenoid analogs or derivatives may have a water solubility of greater than about 10 mg/mL. In certain embodiments, water-soluble carotenoid analogs or derivatives may have a water solubility of greater than about 20 mg/mL. In some embodiments, water-soluble carotenoid analogs or derivatives may have a water solubility of greater than about 50 mg/mL.

Naturally occurring carotenoids such as xanthophyll carotenoids of the C40 series, which includes commercially important compounds such as lutein, zeaxanthin, and astaxanthin, have poor aqueous solubility in the native state. Varying the chemical structure(s) of the esterified moieties may vastly increase the aqueous solubility and/or dispersibility of derivatized carotenoids.

In some embodiments, highly water-dispersible C40 carotenoid derivatives may include natural source RRR-lutein (β,ε-carotene-3,3′-diol) derivatives. Derivatives may be synthesized by esterification with inorganic phosphate and succinic acid, respectively, and subsequently converted to the sodium salts. Deep orange, evenly colored aqueous suspensions were obtained after addition of these derivatives to USP-purified water. Aqueous dispersibility of the disuccinate sodium salt of natural lutein was 2.85 mg/mL; the diphosphate salt demonstrated a >10-fold increase in dispersibility at 29.27 mg/mL. Aqueous suspensions may be obtained without the addition of heat, detergents, co-solvents, or other additives.

The direct aqueous superoxide scavenging abilities of these derivatives were subsequently evaluated by electron paramagnetic resonance (EPR) spectroscopy in a well-characterized in vitro isolated human neutrophil assay. The derivatives may be potent (millimolar concentration) and nearly identical aqueous-phase scavengers, demonstrating dose-dependent suppression of the superoxide anion signal (as detected by spin-trap adducts of DEPMPO) in the millimolar range. Evidence of card-pack aggregation was obtained for the diphosphate derivative with UV-Vis spectroscopy (discussed herein), whereas limited card-pack and/or head-to-tail aggregation was noted for the disuccinate derivative. These lutein-based soft drugs may find utility in those commercial and clinical applications for which aqueous-phase singlet oxygen quenching and direct radical scavenging may be required.

The absolute size of a carotenoid derivative (in 3 dimensions) is important when considering its use in biological and/or medicinal applications. Some of the largest naturally occurring carotenoids are no greater than about C₅₀. This is probably due to size limits imposed on molecules requiring incorporation into and/or interaction with cellular membranes. Cellular membranes may be particularly co-evolved with molecules of a length of approximately 30 nm. In some embodiments, carotenoid derivatives may be greater than or less than about 30 nm in size. In certain embodiments, carotenoid derivatives may be able to change conformation and/or otherwise assume an appropriate shape, which effectively enables the carotenoid derivative to efficiently interact with a cellular membrane.

Although the above structure, and subsequent structures, depict alkenes in the E configuration this should not be seen as limiting. Compounds discussed herein may include embodiments where alkenes are in the Z configuration or include alkenes in a combination of Z and E configurations within the same molecule. The compounds depicted herein may naturally convert between the Z and E configuration and/or exist in equilibrium between the two configurations.

Compounds described herein embrace isomers mixtures, racemic, optically active, and optically inactive stereoisomers and compounds. Carotenoid analogs or derivatives may have increased water solubility and/or water dispersibility relative to some or all known naturally occurring carotenoids. In some embodiments, one or more co-antioxidants may be coupled to a carotenoid or carotenoid derivative or analog.

In some embodiments, carotenoid analogs or derivatives may be employed in “self-formulating” aqueous solutions, in which the compounds spontaneously self-assemble into macromolecular complexes. These complexes may provide stable formulations in terms of shelf life. The same formulations may be parenterally administered, upon which the spontaneous self-assembly is overcome by interactions with serum and/or tissue components in vivo.

Some specific embodiments may include phosphate, succinate, co-antioxidant (e.g., Vitamin C, Vitamin C analogs, Vitamin C derivatives, Vitamin E, Vitamin E analogs, Vitamin E derivatives, or flavonoids), or combinations thereof derivatives or analogs of carotenoids. Flavonoids may include, for example, quercetin, xanthohumol, isoxanthohumol, or genistein. Derivatives or analogs may be derived from any known carotenoid (naturally or synthetically derived). Specific examples of naturally occurring carotenoids which compounds described herein may be derived from include for example zeaxanthin, lutein, lycophyll, astaxanthin, and lycopene.

The synthesis of water-soluble and/or water-dispersible carotenoids (e.g., C40) analogs or derivatives—as potential parenteral agents for clinical applications may improve the injectability of these compounds as therapeutic agents, a result perhaps not achievable through other formulation methods. The methodology may be extended to carotenoids with fewer than 40 carbon atoms in the molecular skeleton and differing ionic character. The methodology may be extended to carotenoids with greater than 40 carbon atoms in the molecular skeleton. The methodology may be extended to non-symmetric carotenoids. The aqueous dispersibility of these compounds allows proof-of-concept studies in model systems (e.g. cell culture), where the high lipophilicity of these compounds previously limited their bioavailability and hence proper evaluation of efficacy. Esterification or etherification may be useful to increase oral bioavailability, a fortuitous side effect of the esterification process, which can increase solubility in gastric mixed micelles. These compounds, upon introduction to the mammalian GI tract, are rapidly and effectively cleaved to the parent, non-esterified compounds, and enter the systemic circulation in that manner and form. The effect of the intact ester and/or ether compound on the therapeutic endpoint of interest can be obtained with parenteral administration of the compound(s). The net overall effect is an improvement in potential clinical utility for the lipophilic carotenoid compounds as therapeutic agents.

In one embodiment, a subject may be administered a pharmaceutical composition comprising a carotenoid analog or derivative. The analog or derivative may be broken down according to the following reaction:

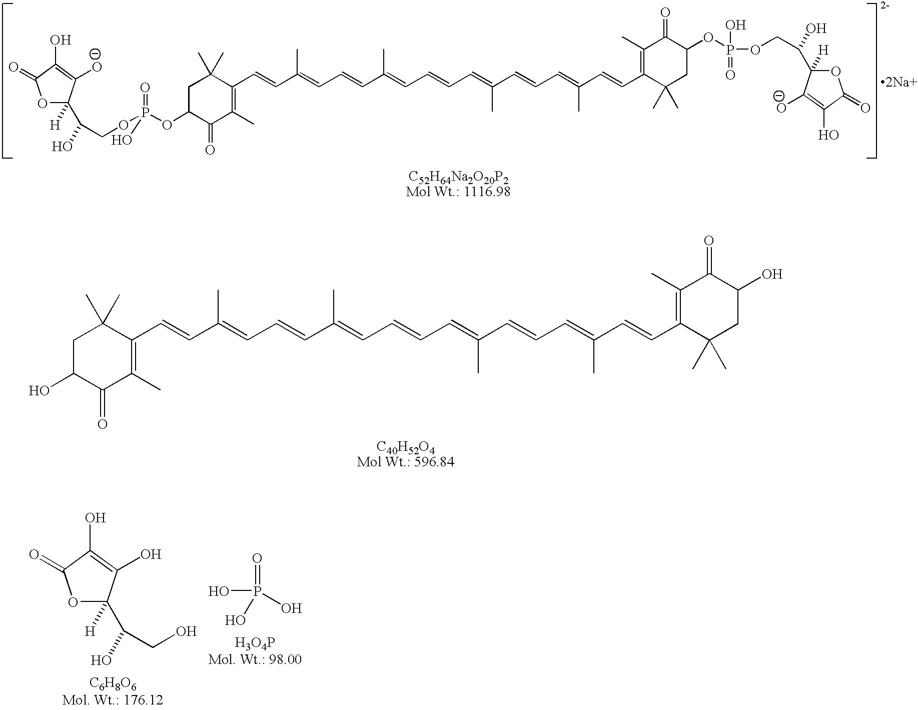

In some embodiments, the principles of retrometabolic drug design may be utilized to produce novel soft drugs from the asymmetric parent carotenoid scaffold (e.g., RRR-lutein (β,ε-carotene-3,3′-diol)). For example, lutein scaffold for derivatization was obtained commercially as purified natural plant source material, and was primarily the RRR-stereoisomer (one of 8 potential stereoisomers). Lutein (Scheme 1) possesses key characteristics—similar to starting material astaxanthin—which make it an ideal starting platform for retrometabolic syntheses: (1) synthetic handles (hydroxyl groups) for conjugation, and (2) an excellent safety profile for the parent compound. As stated above, lutein is available commercially from multiple sources in bulk as primarily the RRR-stereoisomer, the primary isomer in the human diet and human retinal tissue.

In some embodiments, carotenoid analogs or derivatives may have increased water solubility and/or water dispersibility relative to some or all known naturally occurring carotenoids.

In some embodiments, the carotenoid derivatives may include compounds having a structure including a polyene chain (i.e., backbone of the molecule). The polyene chain may include between about 5 and about 15 unsaturated bonds. In certain embodiments, the polyene chain may include between about 7 and about 12 unsaturated bonds. In some embodiments a carotenoid derivative may include 7 or more conjugated double bonds to achieve acceptable antioxidant properties.

In some embodiments, decreased antioxidant properties associated with shorter polyene chains may be overcome by increasing the dosage administered to a subject or patient.

Some embodiments may include solutions or pharmaceutical preparations of carotenoids and/or carotenoid derivatives combined with co-antioxidants, in particular vitamin C and/or vitamin C analogs or derivatives. Pharmaceutical preparations may include about a 2:1 ratio of vitamin C to carotenoid respectively.

In some embodiments, co-antioxidants (e.g., vitamin C) may increase solubility of the chemical compound. In certain embodiments, co-antioxidants (e.g., vitamin C) may decrease toxicity associated with at least some carotenoid analogs or derivatives. In certain embodiments, co-antioxidants (e.g., vitamin C) may increase the potency of the chemical compound synergistically. Co-antioxidants may be coupled (e.g., a covalent bond) to the carotenoid derivative. Co-antioxidants may be included as a part of a pharmaceutically acceptable formulation.

As used herein the terms “structural carotenoid analogs or derivatives” may be generally defined as carotenoids and the biologically active structural analogs or derivatives thereof. “Derivative” in the context of this application is generally defined as a chemical substance derived from another substance either directly or by modification or partial substitution. “Analog” in the context of this application is generally defined as a compound that resembles another in structure but is not necessarily an isomer. Typical analogs or derivatives include molecules which demonstrate equivalent or improved biologically useful and relevant function, but which differ structurally from the parent compounds. Parent carotenoids are selected from the more than 700 naturally occurring carotenoids described in the literature, and their stereo- and geometric isomers. Such analogs or derivatives may include, but are not limited to, esters, ethers, carbonates, amides, carbamates, phosphate esters and ethers, sulfates, glycoside ethers, with or without spacers (linkers).

As used herein the terms “the synergistic combination of more than one xanthophyll carotenoid or structural analog or derivative or synthetic intermediate of carotenoids” may be generally defined as any composition including one xanthophyll carotenoid or a structural carotenoid analog or derivative or synthetic intermediate combined with one or more different xanthophyll carotenoids or structural carotenoid analogs or derivatives or synthetic intermediates or co-antioxidants, either as derivatives or in solutions and/or formulations.

Certain embodiments may include administering a xanthophyll carotenoid or a structural carotenoid analogs or derivatives or synthetic intermediates alone or in combination to a subject such that at least a portion of the adverse effects of COX-2 selective inhibitor drugs are thereby reduced, inhibited and/or ameliorated. The xanthophyll carotenoid or a structural carotenoid analogs or derivatives or synthetic intermediates may be water-soluble and/or water dispersible derivatives. The carotenoid derivatives may include any substituent that substantially increases the water solubility of the naturally occurring carotenoid. The carotenoid derivatives may retain and/or improve the antioxidant properties of the parent carotenoid. The carotenoid derivatives may retain the non-toxic properties of the parent carotenoid. The carotenoid derivatives may have increased bioavailability, relative to the parent carotenoid, upon administration to a subject. The parent carotenoid may be naturally occurring.

Other embodiments may include the administering a composition comprised of the synergistic combination of more than one xanthophyll carotenoids or structural carotenoid analogs or derivatives or synthetic intermediates to a subject such that at least a portion of the adverse effects of COX-2 selective inhibitor drugs are thereby reduced, inhibited and/or ameliorated. The composition may be a “racemic” (i.e. mixture of the potential stereoisomeric forms) mixture of carotenoid derivatives. Included as well are pharmaceutical compositions comprised of structural analogs or derivatives or synthetic intermediates of carotenoids in combination with a pharmaceutically acceptable carrier. In one embodiment, a pharmaceutically acceptable carrier may be serum albumin. In one embodiment, structural analogs or derivatives or synthetic intermediates of carotenoids may be complexed with human serum protein such as, for example, human serum albumin (i.e., HSA) in a solvent. In an embodiment, HSA may act as a pharmaceutically acceptable carrier.

In some embodiments, a single stereoisomer of a structural analog or derivative or synthetic intermediate of carotenoids may be administered to a human subject in order to ameliorate a pathological condition. Administering a single stereoisomer of a particular compound (e.g., as part of a pharmaceutical composition) to a human subject may be advantageous (e.g., increasing the potency of the pharmaceutical composition). Administering a single stereoisomer may be advantageous due to the fact that only one isomer of potentially many may be biologically active enough to have the desired effect.

In some embodiments, compounds described herein may be administered in the form of nutraceuticals. “Nutraceuticals” as used herein, generally refers to dietary supplements, foods, or medical foods that: 1. possess health benefits generally defined as reducing the risk of a disease or health condition, including the management of a disease or health condition or the improvement of health; and 2. are safe for human consumption in such quantity, and with such frequency, as required to realize such properties. Generally a nutraceutical is any substance that is a food or a part of a food and provides medical or health benefits, including the prevention and treatment of disease. Such products may range from isolated nutrients, dietary supplements and specific diets to genetically engineered designer foods, herbal products, and processed foods such as cereals, soups and beverages. It is important to note that this definition applies to all categories of food and parts of food, ranging from dietary supplements such as folic acid, used for the prevention of spina bifida, to chicken soup, taken to lessen the discomfort of the common cold. This definition also includes a bio-engineered designer vegetable food, rich in antioxidant ingredients, and a stimulant functional food or pharmafood. Within the context of the description herein where the composition, use and/or delivery of pharmaceuticals are described nutraceuticals may also be composed, used, and/or delivered in a similar manner where appropriate.

### Dosage and Administration

The xanthophyll carotenoid, carotenoid derivative or analog may be administered at a dosage level up to conventional dosage levels for xanthophyll carotenoids, carotenoid derivatives or analogs, but will typically be less than about 2 gm per day. Suitable dosage levels may depend upon the overall systemic effect of the chosen xanthophyll carotenoids, carotenoid derivatives or analogs, but typically suitable levels will be about 0.001 to 50 mg/kg body weight of the patient per day, from about 0.005 to 30 mg/kg per day, or from about 0.05 to 10 mg/kg per day. The compound may be administered on a regimen of up to 6 times per day, between about 1 to 4 times per day, or once per day.

In the case where an oral composition is employed, a suitable dosage range is, e.g. from about 0.01 mg to about 100 mg of a xanthophyll carotenoid, carotenoid derivative or analog per kg of body weight per day, preferably from about 0.1 mg to about 10 mg per kg and for cytoprotective use from 0.1 mg to about 100 mg of a xanthophyll carotenoid, carotenoid derivative or analog per kg of body weight per day.

It will be understood that the dosage of the therapeutic agents will vary with the nature and the severity of the condition to be treated, and with the particular therapeutic agents chosen. The dosage will also vary according to the age, weight, physical condition and response of the individual patient. The selection of the appropriate dosage for the individual patient is within the skills of a clinician.

In some embodiments, compositions may include all compositions of 1.0 gram or less of a particular structural carotenoid analog, in combination with 1.0 gram or less of one or more other structural carotenoid analogs or derivatives or synthetic intermediates and/or co-antioxidants, in an amount which is effective to achieve its intended purpose. While individual subject needs vary, determination of optimal ranges of effective amounts of each component is with the skill of the art. Typically, a structural carotenoid analog or derivative or synthetic intermediates may be administered to mammals, in particular humans, orally at a dose of 5 to 100 mg per day referenced to the body weight of the mammal or human being treated for a particular disease. Typically, a structural carotenoid analog or derivative or synthetic intermediate may be administered to mammals, in particular humans, parenterally at a dose of between 5 to 1000 mg per day referenced to the body weight of the mammal or human being treated for a particular disease. In other embodiments, about 100 mg of a structural carotenoid analog or derivative or synthetic intermediate is either orally or parenterally administered to treat or prevent disease.

The unit oral dose may comprise from about 0.25 mg to about 1.0 gram, or about 5 to 25 mg, of a structural carotenoid analog. The unit parenteral dose may include from about 25 mg to 1.0 gram, or between 25 mg and 500 mg, of a structural carotenoid analog. The unit intracoronary dose may include from about 25 mg to 1.0 gram, or between 25 mg and 100 mg, of a structural carotenoid analog. The unit doses may be administered one or more times daily, on alternate days, in loading dose or bolus form, or titrated in a parenteral solution to commonly accepted or novel biochemical surrogate marker(s) or clinical endpoints as is with the skill of the art.

In addition to administering a structural carotenoid analog or derivative or synthetic intermediate as a raw chemical, the compounds may be administered as part of a pharmaceutical preparation containing suitable pharmaceutically acceptable carriers, preservatives, excipients and auxiliaries which facilitate processing of the structural carotenoid analog or derivative or synthetic intermediates which may be used pharmaceutically. The preparations, particularly those preparations which may be administered orally and which may be used for the preferred type of administration, such as tablets, softgels, lozenges, dragees, and capsules, and also preparations which may be administered rectally, such as suppositories, as well as suitable solutions for administration by injection or orally or by inhalation of aerosolized preparations, may be prepared in dose ranges that provide similar bioavailability as described above, together with the excipient. While individual needs may vary, determination of the optimal ranges of effective amounts of each component is within the skill of the art.

In some embodiments, the COX-2 selective inhibitor and the xanthophyll carotenoid, carotenoid derivative or analog may be administered separately in separate dosage forms or together in a single unit dosage form. Where separate dosage formulations are used, the xanthophylls carotenoid, carotenoid derivative or analog and the COX-2 selective inhibitor can be administered at essentially the same time, i.e., concurrently, or at separately staggered times, i.e., sequentially, and in any order. In certain embodiments the xanthophyll carotenoid, carotenoid derivative or analog and the COX-2 selective inhibitor can be co-administered concurrently on a once-a-day (QD) dosing schedule; however, varying dosing schedules, such as the xanthophyll carotenoid, carotenoid derivative or analog once per day and the COX-2 selective inhibitor once, twice or more times per day, or the COX-2 selective inhibitor once per day and the xanthophyll carotenoid, carotenoid derivative or analog once, twice or more times per day, is also encompassed herein. According to certain application(s) of the present embodiments, a single oral dosage formulation comprising the xanthophyll carotenoid, carotenoid derivative or analog and the COX-2 selective inhibitor may be preferred. In other embodiments, it may be desirable to administer the xanthophyll carotenoid, carotenoid derivative or analog separately from the COX-2 inhibitor. A single dosage formulation will provide convenience for the patient.

The COX-2 selective inhibitor may be administered at a dosage level up to conventional dosage levels for NSAIDs. Suitable dosage levels will depend upon the anti-inflammatory effect of the chosen inhibitor of cyclooxygenase-2, but typically suitable levels will be between about 0.001 to 50 mg/kg body weight of the patient per day, between about 0.005 to 30 mg/kg per day, or between about 0.05 to 10 mg/kg per day. In some embodiments, the compound may be administered on a regimen of up to 6 times per day, from 1 to 4 times per day, or once per day.

In the case where an oral composition is employed, a suitable dosage range is, e.g. from about 0.01 mg to about 100 mg of a COX-2 selective inhibitor per kg of body weight per day, or from about 0.1 mg to about 10 mg per kg of a COX-2 selective inhibitor per kg of body weight per day.

General guidance in determining effective dose ranges for pharmacologically active compounds and compositions for use in the presently described embodiments may be found, for example, in the publications of the International Conference on Harmonisation and in REMINGTON'S PHARMACEUTICAL SCIENCES, 8^{th} Edition Ed. Bertram G. Katzung, chapters 27 and 28, pp. 484-528 (Mack Publishing Company 1990) and yet further in BASIC & CLINICAL PHARMACOLOGY, chapters 5 and 66, (Lange Medical Books/McGraw-Hill, New York, 2001).

### Pharmaceutical Compositions

Any suitable route of administration may be employed for providing a patient with an effective dosage of drugs of the present invention. For example, oral, rectal, topical, parenteral, ocular, pulmonary, nasal, and the like may be employed. Dosage forms include tablets, troches, dispersions, suspensions, solutions, capsules, creams, ointments, aerosols, and the like. In certain embodiments, it may be advantageous that the compositions described herein be administered orally.

The compositions may include those compositions suitable for oral, rectal, topical, parenteral (including subcutaneous, intramuscular, and intravenous), ocular (ophthalmic), pulmonary (aerosol inhalation), or nasal administration, although the most suitable route in any given case will depend on the nature and severity of the conditions being treated and on the nature of the active ingredient. They may be conveniently presented in unit dosage form and prepared by any of the methods well-known in the art of pharmacy.

For administration by inhalation, the drugs used in the present invention are conveniently delivered in the form of an aerosol spray presentation from pressurized packs or nebulisers. The compounds may also be delivered as powders which may be formulated and the powder composition may be inhaled with the aid of an insulation powder inhaler device.

Suitable topical formulations for use in the present embodiments may include transdermal devices, aerosols, creams, ointments, lotions, dusting powders, and the like.

In practical use, drugs used can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). In preparing the compositions for oral dosage form, any of the usual pharmaceutical media may be employed, such as, for example, water, glycols, oils, alcohols, flavoring agents, preservatives, coloring agents and the like in the case of oral liquid preparations, such as, for example, suspensions, elixirs and solutions; or carriers such as starches, sugars, microcrystalline cellulose, diluents, granulating agents, lubricants, binders, disintegrating agents and the like in the case of oral solid preparations such as, for example, powders, capsules and tablets, with the solid oral preparations being preferred over the liquid preparations. Because of their ease of administration, tablets and capsules represent the most advantageous oral dosage unit form in which case solid pharmaceutical carriers are obviously employed. If desired, tablets may be coated by standard aqueous or nonaqueous techniques.

The pharmaceutical preparations may be manufactured in a manner which is itself known to one skilled in the art, for example, by means of conventional mixing, granulating, dragee-making, softgel encapsulation, dissolving, extracting, or lyophilizing processes. Thus, pharmaceutical preparations for oral use may be obtained by combining the active compounds with solid and semi-solid excipients and suitable preservatives, and/or co-antioxidants. Optionally, the resulting mixture may be ground and processed. The resulting mixture of granules may be used, after adding suitable auxiliaries, if desired or necessary, to obtain tablets, softgels, lozenges, capsules, or dragee cores.

Suitable excipients may be fillers such as saccharides (e.g., lactose, sucrose, or mannose), sugar alcohols (e.g., mannitol or sorbitol), cellulose preparations and/or calcium phosphates (e.g., tricalcium phosphate or calcium hydrogen phosphate). In addition binders may be used such as starch paste (e.g., maize or corn starch, wheat starch, rice starch, potato starch, gelatin, tragacanth, methyl cellulose, hydroxypropylmethylcellulose, sodium carboxymethylcellulose, and/or polyvinyl pyrrolidone). Disintegrating agents may be added (e.g., the above-mentioned starches) as well as carboxymethyl-starch, cross-linked polyvinyl pyrrolidone, agar, or alginic acid or a salt thereof (e.g., sodium alginate). Auxiliaries are, above all, flow-regulating agents and lubricants (e.g., silica, talc, stearic acid or salts thereof, such as magnesium stearate or calcium stearate, and/or polyethylene glycol, or PEG). Dragee cores are provided with suitable coatings, which, if desired, are resistant to gastric juices. Softgelatin capsules (“softgels”) are provided with suitable coatings, which, typically, contain gelatin and/or suitable edible dye(s). Animal component-free and kosher gelatin capsules may be particularly suitable for the embodiments described herein for wide availability of usage and consumption. For this purpose, concentrated saccharide solutions may be used, which may optionally contain gum arabic, talc, polyvinyl pyrrolidone, polyethylene glycol (PEG) and/or titanium dioxide, lacquer solutions and suitable organic solvents or solvent mixtures, including dimethylsulfoxide (DMSO), tetrahydrofuran (THF), acetone, ethanol, or other suitable solvents and co-solvents. In order to produce coatings resistant to gastric juices, solutions of suitable cellulose preparations such as acetylcellulose phthalate or hydroxypropylmethyl-cellulose phthalate, may be used. Dye stuffs or pigments may be added to the tablets or dragee coatings or softgelatin capsules, for example, for identification or in order to characterize combinations of active compound doses, or to disguise the capsule contents for usage in clinical or other studies.

Other pharmaceutical preparations that may be used orally include push-fit capsules made of gelatin, as well as soft, thermally sealed capsules made of gelatin and a plasticizer such as glycerol or sorbitol. The push-fit capsules may contain the active compounds in the form of granules that may be mixed with fillers such as, for example, lactose, binders such as starches, and/or lubricants such as talc or magnesium stearate and, optionally, stabilizers and/or preservatives. In soft capsules, the active compounds may be dissolved or suspended in suitable liquids, such as fatty oils such as rice bran oil or peanut oil or palm oil, or liquid paraffin. In some embodiments, stabilizers and preservatives may be added.

In some embodiments, pulmonary administration of a pharmaceutical preparation may be desirable. Pulmonary administration may include, for example, inhalation of aerosolized or nebulized liquid or solid particles of the pharmaceutically active component dispersed in and surrounded by a gas.

Possible pharmaceutical preparations, which may be used rectally, include, for example, suppositories, which consist of a combination of the active compounds with a suppository base. Suitable suppository bases are, for example, natural or synthetic triglycerides, or paraffin hydrocarbons. In addition, it is also possible to use gelatin rectal capsules that consist of a combination of the active compounds with a base. Possible base materials include, for example, liquid triglycerides, polyethylene glycols, or paraffin hydrocarbons.

Suitable formulations for parenteral administration include, but are not limited to, aqueous solutions of the active compounds in water-soluble and/or water dispersible form, for example, water-soluble salts, esters, carbonates, phosphate esters or ethers, sulfates, glycoside ethers, together with spacers and/or linkers. Suspensions of the active compounds as appropriate oily injection suspensions may be administered, particularly suitable for intramuscular injection. Suitable lipophilic solvents, co-solvents (such as DMSO or ethanol), and/or vehicles including fatty oils, for example, rice bran oil or peanut oil and/or palm oil, or synthetic fatty acid esters, for example, ethyl oleate or triglycerides, may be used. Aqueous injection suspensions may contain substances that increase the viscosity of the suspension including, for example, sodium carboxymethyl cellulose, sorbitol, dextran, and/or cyclodextrins. Cyclodextrins (e.g., β-cyclodextrin) may be used specifically to increase the water solubility for parenteral injection of the structural carotenoid analog. Liposomal formulations, in which mixtures of the structural carotenoid analog or derivative with, for example, egg yolk phosphotidylcholine (E-PC), may be made for injection. Optionally, the suspension may contain stabilizers, for example, antioxidants such as BHT, and/or preservatives, such as benzyl alcohol.

The compounds of this invention can be administered in such oral dosage forms as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular form, all using dosage forms well known to those of ordinary skill in the pharmaceutical arts. They can be administered alone, but generally will be administered with a pharmaceutical carrier selected on the basis of the chosen route of administration and standard pharmaceutical practice.

The dosage regimen for the compounds of the present invention will, of course, vary depending upon known factors, such as the pharmacodynamic characteristics of the particular agent and its mode and route of administration; the species, age, sex, health, medical condition, and weight of the recipient; the nature and extent of the symptoms; the kind of concurrent treatment; the frequency of treatment; the route of administration, the renal and hepatic function of the patient, and the effect desired. A physician or veterinarian can determine and prescribe the effective amount of the drug required to prevent, counter, or arrest the progress or the development of cardiovascular complications associated with the a administration of COX-2 selective inhibitor drugs.

By way of general guidance, the daily oral dosage of each active ingredient, when used for the indicated effects, will range between about 0.001 to 1000 mg/kg of body weight, between about 0.01 to 100 mg/kg of body weight per day, or between about 1.0 to 20 mg/kg/day. Intravenously administered doses may range from about 1 to about 10 mg/kg/minute during a constant rate infusion. Compounds of this invention may be administered in a single daily dose, or the total daily dosage may be administered in divided doses of two, three, or four or more times daily.

The pharmaceutical compositions described herein may further be administered in intranasal form via topical use of suitable intranasal vehicles, or via transdermal routes, using transdermal skin patches. When administered in the form of a transdermal delivery system, the dosage administration will, of course, be continuous rather than intermittent throughout the dosage regimen.

The compounds are typically administered in admixture with suitable pharmaceutical diluents, excipients, or carriers (collectively referred to herein as “pharmacologically inert carriers”) suitably selected with respect to the intended form of administration, that is, oral tablets, capsules, elixirs, syrups and the like, and consistent with conventional pharmaceutical practices.

For instance, for oral administration in the form of a tablet or capsule, the pharmacologically active component may be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

The compounds of the present invention may also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Compounds of the present invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxide-polylysine substituted with palmitoyl residues. Furthermore, the compounds of the present invention may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels.

Dosage forms (pharmaceutical compositions) suitable for administration may contain from about 1 milligram to about 100 milligrams or more of active ingredient per dosage unit. In these pharmaceutical compositions the active ingredient will ordinarily be present in an amount of about 0.5-95% by weight based on the total weight of the composition.

Gelatin capsules may contain the active ingredient and powdered carriers, such as lactose, starch, cellulose derivatives, magnesium stearate, stearic acid, and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract.

Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

Suitable pharmaceutical carriers are described in Remington's Pharmaceutical Sciences, Mack Publishing Company, a standard reference text in this field.

### EXAMPLES

Having now described the invention, the same will be more readily understood through reference to the following example(s), which are provided by way of illustration, and are not intended to be limiting of the present invention.

General. Natural source lutein (90%) was obtained from ChemPacific, Inc. (Baltimore, Md.) as a red-orange solid and was used without further purification. All other reagents and solvents used were purchased from Acros (New Jersey, USA) and were used without further purification. All reactions were performed under N₂ atmosphere. All flash chromatographic purifications were performed on Natland International Corporation 230-400 mesh silica gel using the indicated solvents. LC/MS (APCI) and LC/MS (ESI) were recorded on an Agilent 1100 LC/MSD VL system; column: Zorbax Eclipse XDB-C18 Rapid Resolution (4.6×75 mm, 3.5 μm, USUT002736); temperature: 25° C.; starting pressure: 105 bar; flow rate: 1.0 mL/min; mobile phase (% A=0.025% trifluoroacetic acid in H₂O, % B=0.025% trifluoroacetic acid in acetonitrile) Gradient program: 70% A/30% B (start), step gradient to 50% B over 5 min, step gradient to 98% B over 8.30 min, hold at 98% B over 25.20 min, step gradient to 30% B over 25.40 min; PDA Detector: 470 nm. The presence of trifluoroacetic acid in the LC eluents acts to protonate synthesized lutein disuccinate and diphosphate salts to give the free di-acid forms, yielding M⁺=768 for the disuccinate salt sample and M⁺=728 for the diphosphate salt sample in MS analyses. LRMS: +mode; ESI: electrospray chemical ionization, ion collection using quadrapole; APCI: atmospheric pressure chemical ionization, ion collection using quadrapole. MS (ESI-IT) was recorded on a HCT plus Bruker Daltonics Mass Spectrometer system, LRMS: + mode; ESI-IT: electrospray chemical ionization, ion collection using ion trap. ¹H NMR analyses were attempted on Varian spectrometers (300 and 500 MHz). NMR analyses of natural source lutein as well as synthesized lutein derivatives yielded only partially discernable spectra, perhaps due to the presence of interfering impurities (natural source lutein), or due to aggregation (natural source lutein and derivatives). In attempts to circumvent the problems associated with NMR analyses, samples were prepared using mixtures of deuterated solvents including methanol/chloroform, methanol/water, methyl sulfoxide/water, and chloroform/methanol/water. However, such attempts failed to give useful data.

Natural source lutein (β,ε-carotene-3,3′-diol), 1. LC/MS (ESI): 9.95 min (2.78%), λₘₐₓ 226 nm (17%), 425 nm (100%); 10.58 min (3.03%), λₘₐₓ 225 nm (21%), 400 nm (100%); 11.10 min (4.17%), λₘₐₓ 225 nm (16%), 447 nm (100%); 12.41 min (90.02%), λₘₐₓ 269 nm (14%), 447 nm (100%), m/z 568 M⁺ (69%), 551 [M−H₂O+H]⁺ (100%), 533 [M−2H₂O+H]⁺ (8%)

β,ε-carotenyl 3,3′-disuccinate, 2. To a solution of natural source lutein (1) (0.50 g, 0.879 mmol) in CH₂Cl₂ (8 mL) was added N,N-diisopropylethylamine (3.1 mL, 17.58 mmol) and succinic anhydride (0.88 g, 8.79 mmol). The solution was stirred at RT overnight and then diluted with CH₂Cl₂ and quenched with water/1 M HCl (5/1). The aqueous layer was extracted two times with CH₂Cl₂ and the combined organic layer was washed three times with cold water/1 M HCl (5/1), dried over Na₂SO₄, and concentrated. The resulting red-orange oil was washed (slurried) three times with hexanes to yield disuccinate 2 (0.433 g, 64%) as a red-orange solid; LC/MS (APCI): 10.37 min (4.42%), λₘₐₓ 227 nm (56%), 448 nm (100%), m/z 769 [M+H]⁺ (8%), 668 [M−C₄O₃H₄]⁺ (9%), 637 (36%), 138 (100%); 11.50 min (92.40%), λₘₐₓ 269 nm (18%), 447 nm (100%), m/z 769 [M+H]⁺ (7%), 668 [M−C₄O₃H₄]⁺ (9%), 651 (100%); 12.03 min (3.18%) λₘₐₓ 227 nm (55%), 446 nm (100%), m/z 668 [M−C₄O₃H₄]⁺ (15%), 550 (10%), 138 (100%)

β,ε-carotenyl 3,3′-disuccinate sodium salt, 3. To a solution of disuccinate 2 (0.32 g, 0.416 mmol) in CH₂Cl₂/methanol (5 mL/1 mL) at 0° C. was added drop-wise sodium methoxide (25% wt in methanol; 0.170 mL, 0.748 mmol). The solution was stirred at RT overnight and then quenched with water and stirred for 5 min. The solution was then concentrated and the aqueous layer was washed four times with Et₂O. Lyophilization of the clear, red-orange aqueous solution yielded 3 (0.278 g, 91%) as an orange, hygroscopic solid; LC/MS (APCI): 11.71 min (94.29%), λₘₐₓ 269 nm (18%), 446 nm (100%), m/z 769 [M−2Na+3H]⁺ (8%), 668 [M−2Na+2H−C₄O₃H₄]⁺ (6%), 651 (100%); 12.74 min (5.71%), λₘₐₓ 227 nm (30%), 269 nm (18%), 332 nm (39%), 444 nm (100%), m/z 768 [M−2Na+2H]⁺ (2%), 668 [M−2Na+2H−C₄O₃H₄]⁺ (3%), 651 (12%), 138 (100%)

Tribenzyl phosphite, 4. To a well-stirred solution of phosphorus trichloride (1.7 mL, 19.4 mmol) in Et₂O (430 mL) at 0° C. was added dropwise a solution of triethylamine (8.4 mL, 60.3 mmol) in Et₂O (20 mL), followed by a solution of benzyl alcohol (8.1 mL, 77.8 mmol) in Et₂O (20 mL). The mixture was stirred at 0° C. for 30 min and then at RT overnight. The mixture was filtered and the filtrate concentrated to give a colorless oil. Silica chromatography (hexanes/Et₂O/triethylamine, 4/1/1%) of the crude product yielded 4 (5.68 g, 83%) as a clear, colorless oil that was stored under N₂ at −20° C.; ¹H NMR: δ 7.38 (15H, m), 4.90 (6H, d)

Dibenzyl phosphoroiodidate, 5. To a solution of tribenzyl phosphite (5.43 g, 15.4 mmol) in CH₂Cl₂ (8 mL) at 0° C. was added 12 (3.76 g, 14.8 mmol). The mixture was stirred at 0° C. for 10 min or until the solution became clear and colorless. The solution was then stirred at RT for 10 min and used directly in the next step.

3-(Bis benzyl-phosphoryloxy)-3′-(phosphoryloxy)-β,ε-carotene, 6. To a solution of natural source lutein (1) (0.842 g, 1.48 mmol) in CH₂Cl₂ (8 mL) was added pyridine (4.8 mL, 59.2 mmol). The solution was stirred at 0° C. for 5 min and then freshly prepared 5 (14.8 mmol) in CH₂Cl₂ (8 mL) was added drop-wise to the mixture at 0° C. The solution was stirred at 0° C. for 1 h and then diluted with CH₂Cl₂ and quenched with brine. The aqueous layer was extracted twice with CH₂Cl₂ and the combined organic layer was washed once with brine, then dried over Na₂SO₄ and concentrated. Pyridine was removed from the crude red oil by azeotropic distillation using toluene. The crude product was alternately washed (slurried) twice with hexanes and Et₂O to yield 6 as a red oil, used in the next step without further purification; LC/MS (ESI): 9.93 min (44.78%), λₘₐₓ 267 nm (33%), 444 nm (100%), m/z 890 [M−H₂O]⁺ (8%), 811 [M−PO₃H−H₂O+H]⁺ (73%), 533 (100%); 9.99 min (29.0%), λₘₐₓ 268 nm (24%), 446 nm (100%), m/z 890 [M−H₂O]⁺ (6%), 811 [M−PO₃H−H₂O+H]⁺ (72%), 533 (100%); 10.06 min (26.23%), λₘₐₓ 266 nm (15%), 332 nm (22%), 444 nm (100%), m/z 890 [M−H₂O]⁺ (5%), 811 [M−PO₃H−H₂O+H]⁺ (90%), 533 (100%)

3-(Bis benzyl-phosphoryloxy)-3′-hydroxy-β,ε-carotene, 7. To a solution of 6 (0.033 mmol) in tetrahydrofuran/water (1 mL 0.5 mL) at 0° C. was added LiOH—H₂O (0.003 g, 0.073 mmol). The solution was stirred at RT for 1 h and then quenched with methanol. The crude reaction mixture was analyzed by LC/MS; LC/MS (ESI): 10.02 min (40.60%), λₘₐₓ 266 nm (12%), 333 nm (25%), 445 nm (100%), m/z 890 [M−H₂O]⁺ (33%), 811 [M−PO₃H−H₂O+H]⁺ (50%), 533 (100%); 16.37 min (49.56%) λₘₐₓ 267 nm (16%), 332 nm (27%), 446 nm (100%), m/z 828 M⁺ (55%), 550 (44%)

3,3′-Diphosphoryloxy-β,ε-carotene, 8. To a solution of 6 (1.48 mmol) in CH₂Cl₂ (10 mL) at 0° C. was added drop-wise N,O-bis(trimethylsilyl)acetamide (3.7 mL, 14.8 mmol) and then bromotrimethylsilane (1.56 mL, 11.8 mmol). The solution was stirred at 0° C. for 1 h, quenched with methanol, diluted with CH₂Cl₂, and then concentrated. The resulting red oil was alternately washed (slurried) three times with ethyl acetate and CH₂Cl₂ to yield crude phosphate 8 (2.23 g) as a dark orange oil, used in the next step without further purification; LC/MS (ESI): 8.55 min (45.67%), λₘₐₓ 214 nm (25%), 268 nm (28%), 447 nm (100%), m/z 631 [M−PO₃H−H₂O+H]⁺ (30%), 533 (18%), 279 (13%), 138 (87%); 8.95 min (35.0%), λₘₐₓ 217 nm (14%), 268 nm (23%), 448 nm (100%), m/z 631 [M−PO₃H−H₂O+H]⁺ (26%), 533 (32%), 279 (18%), 138 (100%); 9.41 min (9.70%), λₘₐₓ 225 nm (37%), 269 nm (23%), 335 nm (19%), 447 nm (100%), m/z 631 [M−PO₃H−H₂O+H]⁺ (6%), 533 (18%), 279 (13%), 138 (100%)

3,3′-Diphosphoryloxy-β,ε-carotene sodium salt, 9. To a solution of crude 8 (ca 50%; 2.23 g, 3.06 mmol) in methanol (20 mL) at 0° C. was added drop-wise sodium methoxide (25%; 3.5 mL, 15.3 mmol). The solution was stirred at RT for 2 h and the resulting orange solid was washed (slurried) three times with methanol. Water was added to the moist solid and the resulting aqueous layer was extracted with CH₂Cl₂, ethyl acetate, and again with CH₂Cl₂.

Lyophilization of the clear, red-orange aqueous solution yielded 9 (0.956 g, 80% over 3 steps) as an orange, hygroscopic solid; LC/MS (ESI): 7.81 min (22.34%), 1215 nm (34%), 268 nm (30%), 448 nm (100%), m/z 711 [M−4Na−H₂O+5H]⁺ (9%), 533 (13%), 306 (100%); 8.33 min (39.56%), λₘₐₓ 217 nm (14%), 268 nm (20%), 448 nm (100%), m/z 711 [M−4Na−H₂O+5H]⁺ (10%), 533 (11%), 306(100%); 8.90 min (38.09%), λₘₐₓ 223 nm (45%), 269 nm (30%), 336 nm (26%), 448 nm (100%), m/z 711 [M−4Na−H₂O+5H]⁺ (8%), 631 [M−4Na−PO₃H−H₂O+5H]⁺ (18%), 533 (20%), 306 (100%); MS (ESI-IT): m/z 816 M⁺ (55%), 772 [M−2Na+2H]⁺ (37%), 728 [M−4Na+4H]⁺ (74%)

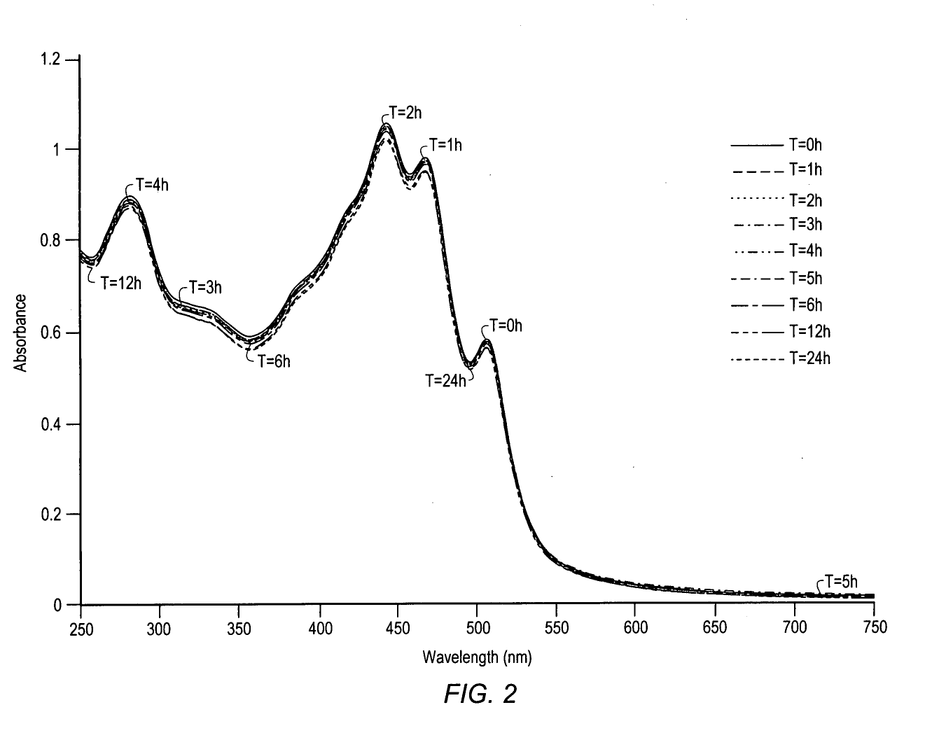

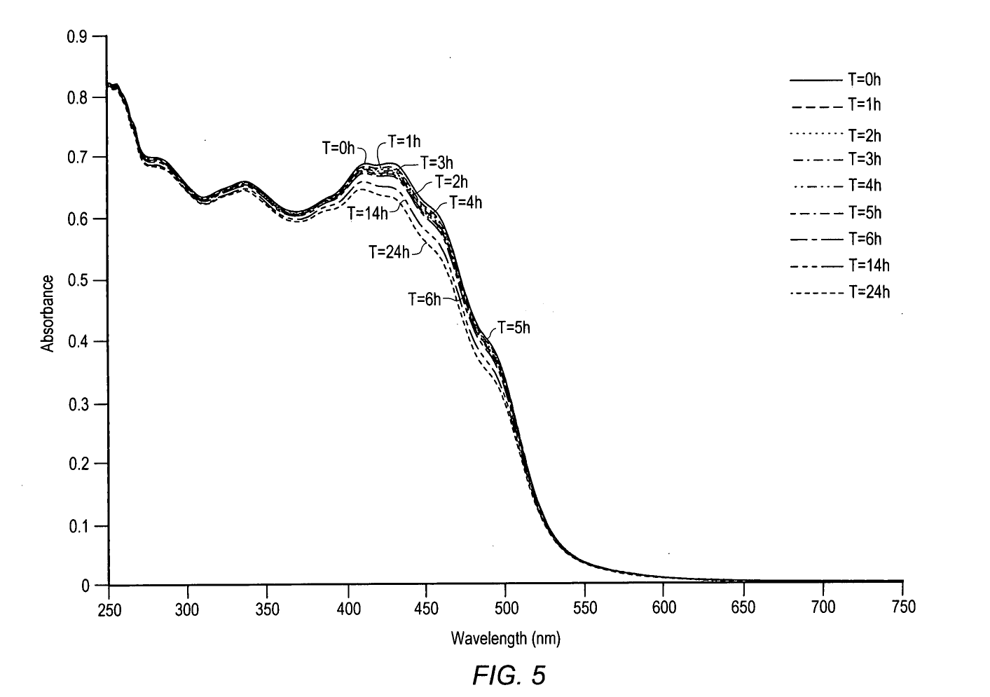

UV/Visible spectroscopy. For spectroscopic sample preparations, 3 and 9 were dissolved in the appropriate solvent to yield final concentrations of approximately 0.01 mM and 0.2 mM, respectively. The solutions were then added to a rectangular cuvette with 1 cm path length fitted with a glass stopper. The absorption spectrum was subsequently registered between 250 and 750 nm. All spectra were accumulated one time with a bandwidth of 1.0 nm at a scan speed of 370 nm/min. For the aggregation time-series measurements, spectra were obtained at baseline (immediately after solvation; time zero) and then at the same intervals up to and including 24 hours post-solvation (see FIG. 2-FIG. 7). Concentration was held constant in the ethanolic titration of the diphosphate lutein sodium salt, for which evidence of card-pack aggregation was obtained (FIG. 5-FIG. 7).

Determination of aqueous solubility/dispersibility. 30.13 mg of 3 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/Vis spectroscopy at 436 nm, and the absorbance was compared to a standard curve compiled from 4 standards of known concentration. The concentration of the original supernatant was calculated to be 2.85 mg/mL and the absorptivity was 36.94 AU*mL/cm*mg. Slight error may have been introduced by the small size of the original aliquot.

Next, 30.80 mg of 9 was added to 1 mL of USP-purified water. The sample was rotated for 2 hours, then centrifuged for 5 minutes. After centrifuging, solid was visible in the bottom of the tube. A 125-μL aliquot of the solution was then diluted to 25 mL. The sample was analyzed by UV/Vis spectroscopy at 411 nm, and the absorbance was compared to a standard curve compiled from 4 standards of known concentration. The concentration of the original supernatant was calculated to be 29.27 mg/mL and the absorptivity was 2.90 AU*mL/cm*mg. Slight error may have been introduced by the small size of the original aliquot.

Leukocyte Isolation and Preparation. Human polymorphonuclear leukocytes (PMNs) were isolated from freshly sampled venous blood of a single volunteer (S.F.L.) by Percoll density gradient centrifugation as described previously. Briefly, each 10 mL of whole blood was mixed with 0.8 mL of 0.1 M EDTA and 25 mL of saline. The diluted blood was then layered over 9 mL of Percoll at a specific density of 1.080 g/mL. After centrifugation at 400×g for 20 min at 20° C., the plasma, mononuclear cell, and Percoll layers were removed. Erythrocytes were subsequently lysed by addition of 18 mL of ice-cold water for 30 s, followed by 2 mL of 10×PIPES buffer (25 mM PIPES, 110 mM NaCl, and 5 mM KCl, titrated to pH 7.4 with NaOH). Cells were then pelleted at 4° C., the supernatant was decanted, and the procedure was repeated. After the second hypotonic cell lysis, cells were washed twice with PAG buffer [PIPES buffer containing 0.003% human serum albumin (HSA) and 0.1% glucose]. Afterward, PMNs were counted by light microscopy on a hemocytometer. The isolation yielded PMNs with a purity of >95%. The final pellet was then suspended in PAG-CM buffer (PAG buffer with 1 mM CaCl₂ and 1 mM MgCl₂).

EPR Measurements. All EPR measurements were performed using a Bruker ER 300 EPR spectrometer operating at X-band with a TM₁₁₀ cavity as previously described. The microwave frequency was measured with a Model 575 microwave counter (EIP Microwave, Inc., San Jose, Calif.). To measure superoxide anion [symbol] generation from phorbol-ester (PMA)-stimulated PMNs, EPR spin-trapping studies were performed using the spin trap DEPMPO (Oxis, Portland, Oreg.) at 10 mM. 1×10⁶ PMNs were stimulated with PMA (1 ng/mL) and loaded into capillary tubes for EPR measurements. To determine the radical scavenging ability of 3 and 9 in aqueous and ethanolic formulations, PMNs were pre-incubated for 5 minutes with test compound, followed by PMA stimulation.

Instrument settings used in the spin-trapping experiments were as follows: modulation amplitude, 0.32 G; time constant, 0.16 s; scan time, 60 s; modulation frequency, 100 kHz; microwave power, 20 milliwatts; and microwave frequency, 9.76 GHz. The samples were placed in a quartz EPR flat cell, and spectra were recorded. The component signals in the spectra were identified and quantified as reported previously.

### UV/Vis Spectral Properties in Organic and Aqueous Solvents.

UV-Vis spectral evaluation of the disuccinate lutein sodium salt is depicted in FIG. 2-FIG. 4. FIG. 2 depicts a time series of the UV/Vis absorption spectra of the disodium disuccinate derivative of natural source lutein in water. The λₘₐₓ (443 nm) obtained at time zero did not appreciably blue-shift over the course of 24 hours, vibrational fine structure was maintained (% III/II=35%), and the spectra became only slightly hypochromic (i.e. decreased in absorbance intensity) over time, indicating minimal time-dependent supramolecular assembly (aggregation) of the card-pack type during this time period. Existence of head-to-tail (J-type) aggregation in solution cannot be ruled out.

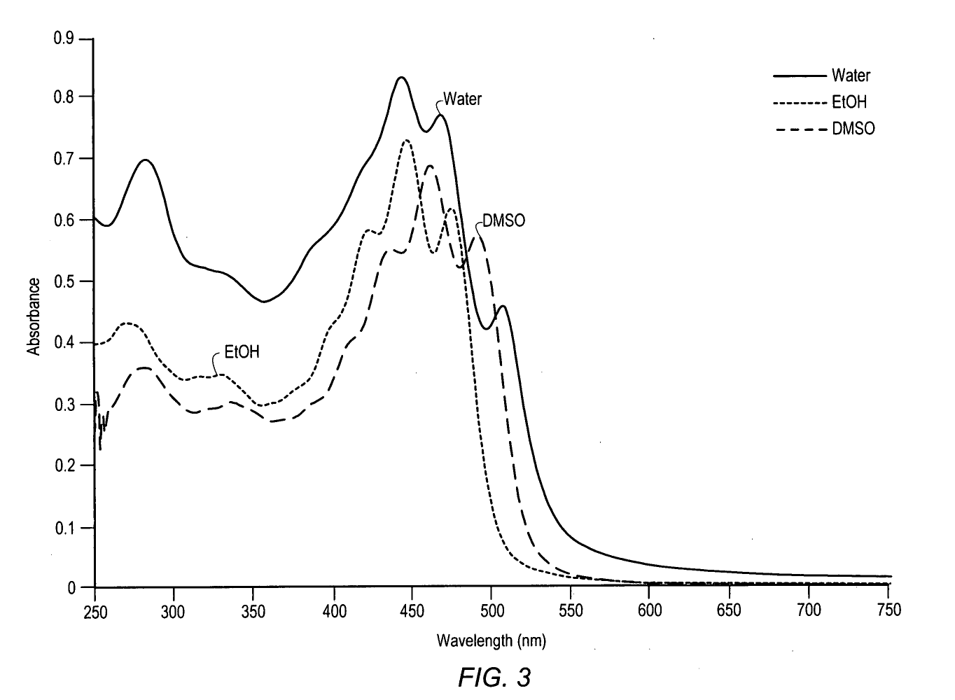

FIG. 3 depicts a UV/Vis absorption spectra of the disodium disuccinate derivative of natural source lutein in water (λₘₐₓ=443 nm), ethanol (λₘₐₓ=446 nm), and DMSO (λₘₐₓ=461 nm). Spectra were obtained at time zero. A prominent cis peak is seen with a maximum at 282 nm in water. The expected bathochromic shift of the spectrum in the more polarizable solvent (DMSO) is seen (461 nm). Only a slight hypsochromic shift is seen between the spectrum in water and that in ethanol, reflecting minimal card-pack aggregation in aqueous solution. Replacement of the main visible absorption band observed in EtOH by an intense peak in the near UV region—narrow and displaying no vibrational fine structure—is not observed in the aqueous solution of this highly water-dispersible derivative, in comparison to the spectrum of pure lutein in an organic/water mixture.

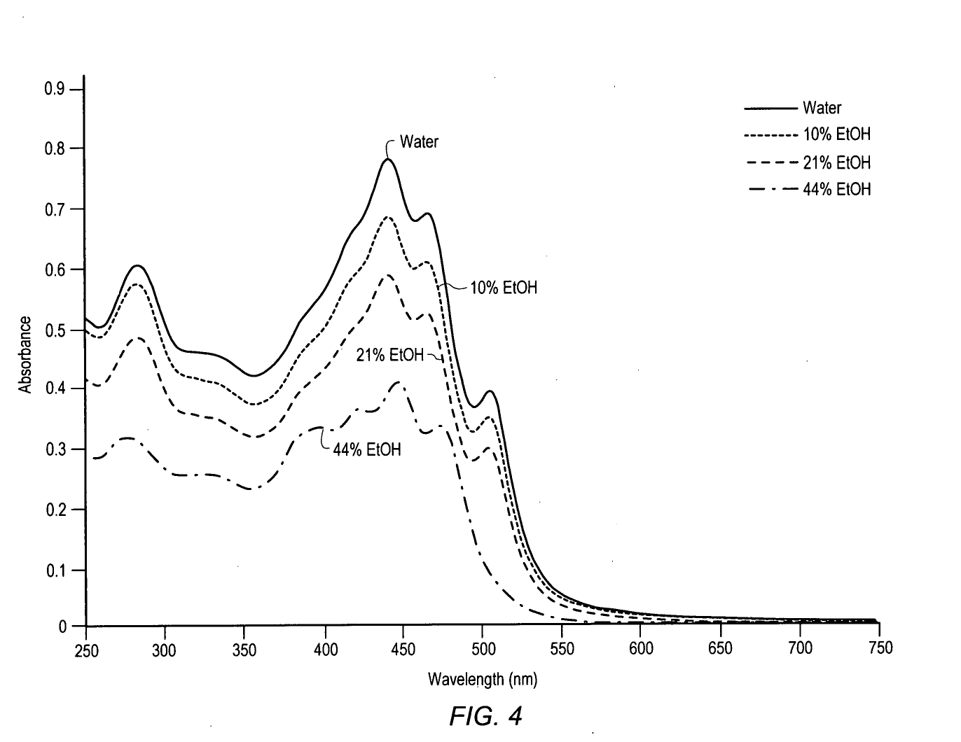

FIG. 4 depicts a UV/Vis absorption spectra of the disodium disuccinate derivative of natural source lutein in water (λₘₐₓ=442 nm) with increasing concentrations of ethanol. The λₘₐₓ increases to 446 nm at an EtOH concentration of 44%, at which point no further shift of the absorption maximum occurs (i.e. a molecular solution has been achieved), identical to that obtained in 100% EtOH (See FIG. 3).

UV-Vis spectral evaluation of the diphosphate lutein sodium salt is depicted in FIG. 5-FIG. 7. FIG. 5 depicts a time series of the UV/Vis absorption spectra of the disodium diphosphate derivative of natural source lutein in water. Loss of vibrational fine structure (spectral distribution beginning to approach unimodality) and the blue-shifted lambda max relative to the lutein chromophore in EtOH suggested that card-pack aggregation was present immediately upon solvation. The λₘₐₓ (428 nm) obtained at time zero did not appreciably blue-shift over the course of 24 hours, and the spectra became slightly more hypochromic over time (i.e. decreased in absorbance intensity), indicating additional time-dependent supramolecular assembly (aggregation) of the card-pack type during this time period. This spectrum was essentially maintained over the course of 24 hours (compare with FIG. 2, disuccinate lutein sodium salt).

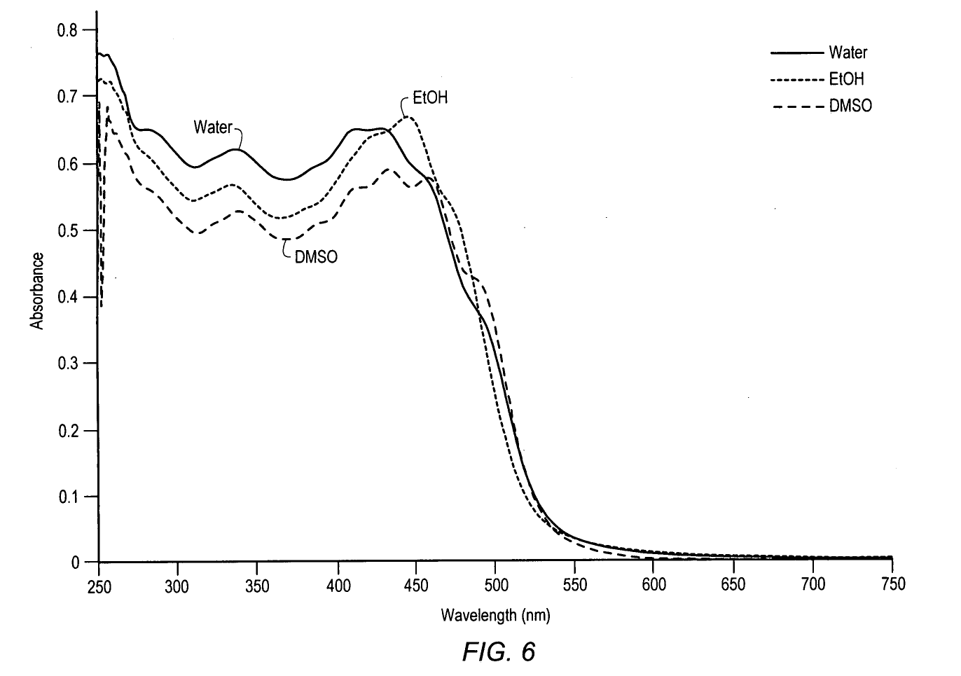

FIG. 6 depicts a UV/Vis absorption spectra of the disodium diphosphate derivative of natural source lutein in 95% ethanol (λₘₐₓ=446 nm), 95% DMSO (λₘₐₓ=459 nm), and water (λₘₐₓ=428 nm). A red-shift was observed (λₘₐₓ to 446 nm), as was observed with the disuccinate derivate. Wetting of the diphosphate lutein derivative with a small amount of water was required to obtain appreciable solubility in organic solvent (e.g. EtOH and DMSO). Spectra were obtained at time zero. The expected bathochromic shift (in this case to 459 nm) of the spectrum in the more polarizable solvent (95% DMSO) is seen. Increased vibrational fine structure and red-shifting of the spectra were observed in the organic solvents.

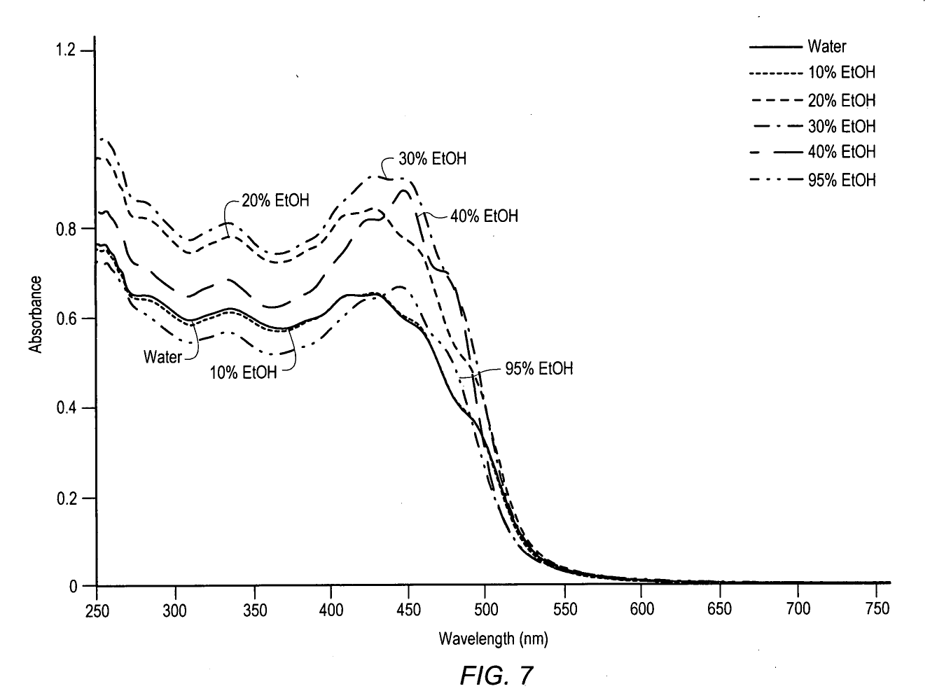

FIG. 7 depicts a UV/Vis absorption spectra of the disodium diphosphate derivative of natural source lutein in water (λₘₐₓ=428 nm) with increasing concentrations of ethanol. Concentration of the derivative was held constant for each increased concentration of EtOH in solution. The λₘₐₓ increases to 448 nm at an EtOH concentration of 40%, at which no further shift of the absorption maximum occurs (i.e. a molecular solution is reached).

### Direct Superoxide Anion Scavenging by EPR Spectroscopy

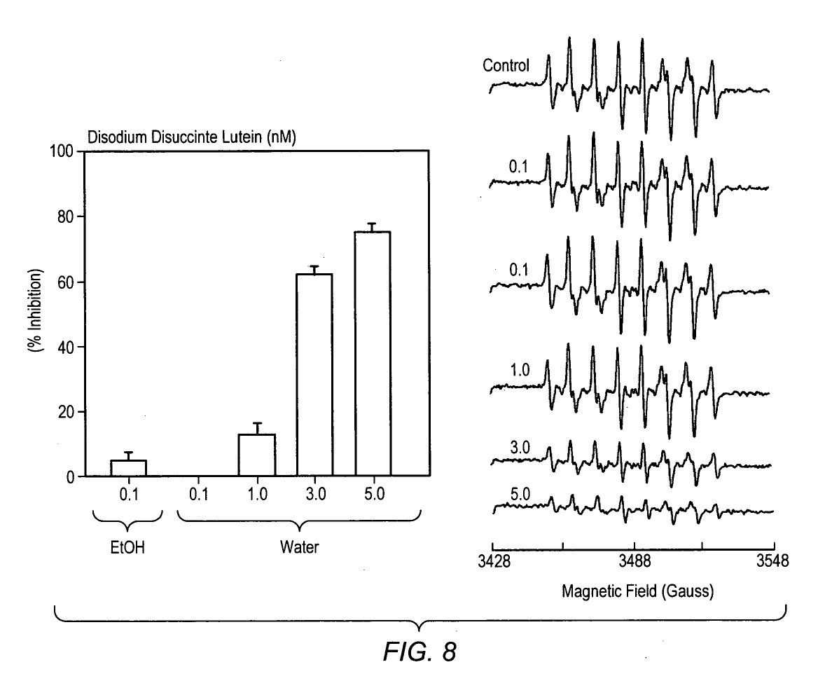

The mean percent inhibition of superoxide anion signal (±SEM) as detected by DEPMPO spin-trap by the disodium disuccinate derivative of natural source lutein (tested in water) is shown in FIG. 8. A 100 μM formulation (0.1 mM) was also tested in 40% EtOH, a concentration shown to produce a molecular (i.e. non-aggregated) solution. As the concentration of the derivative increased, inhibition of superoxide anion signal increased in a dose-dependent manner. At 5 mM, approximately ¾ (75%) of the superoxide anion signal was inhibited. No significant scavenging (0% inhibition) was observed at 0.1 mM in water. Addition of 40% EtOH to the derivative solution at 0.1 mM did not significantly increase scavenging over that provided by the EtOH vehicle alone (5% inhibition). The millimolar concentration scavenging by the derivative was accomplished in water alone, without the addition of organic co-solvent (e.g., acetone, EtOH), heat, detergents, or other additives. This data suggested that card-pack aggregation for this derivative was not occurring in aqueous solution (and thus limiting the interaction of the aggregated carotenoid derivative with aqueous superoxide anion).

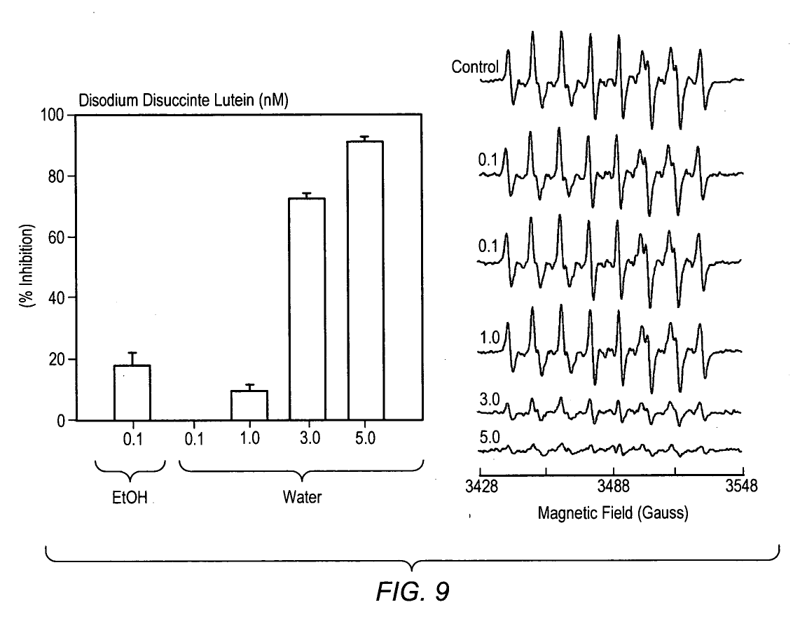

The mean percent inhibition of superoxide anion signal (±SEM) as detected by DEPMPO spin-trap by the disodium diphosphate derivative of natural source lutein (tested in water) is shown in FIG. 9. A 100 μM formulation (0.1 mM) was also tested in 40% EtOH, a concentration also shown to produce a molecular (i.e. non-aggregated) solution of this derivative. As the concentration of the derivative increased, inhibition of the superoxide anion signal increased in a dose-dependent manner. At 5 mM, slightly more than 90% of the superoxide anion signal was inhibited (versus 75% for the disuccinate lutein sodium salt). As for the disuccinate lutein sodium salt, no apparent scavenging (0% inhibition) was observed at 0.1 mM in water. However, a significant increase over background scavenging by the EtOH vehicle (5%) was observed after the addition of 40% EtOH, resulting in a mean 18% inhibition of superoxide anion signal. This suggested that disaggregation of the compound lead to an increase in scavenging ability by this derivative, pointing to slightly increased scavenging ability of molecular solutions of the more water-dispersible diphosphate derivative relative to the disuccinate derivative. Again, the millimolar concentration scavenging by the derivative was accomplished in water alone, without the addition of organic co-solvent (e.g., acetone, EtOH), heat, detergents, or other additives.

TABLE I
Descriptive statistics of mean % inhibition of superoxide anion signal for aqueous and ethanolic
(40%) formulations of disodium disuccinate derivatives of natural source lutein tested in
the current study. Sample sizes of 3 were evaluated for each formulation, with the exception
of natural source lutein in 40% EtOH stock solution (N = 1). Mean % inhibition did not
increase over background levels until sample concentration reached 1 mM in water; likewise,
addition of 40% EtOH at the 0.1 mM concentration did not increase scavenging over background
levels attributable to the EtOH vehicle (mean = 5% inhibition).

| | | | | Mean (% | | | | | |
|---|---|---|---|---|---|---|---|---|---|
| Sample | Solvent | Concentration | N | inhibition) | S.D. | SEM | Min | Max | Range |
| Lutein | 40% | 0.1 mM | 3 | 5.0 | 4.4 | 2.5 | 0 | 8 | 8 |
| Disuccinate | EtOH | | | | | | | | |
| Sodium Salt | | | | | | | | | |
| Lutein | Water | 0.1 mM | 1 | 0.0 | ND | ND | 0 | 0 | 0 |
| Disuccinate | | | | | | | | | |
| Sodium Salt | | | | | | | | | |
| Lutein | Water | 1.0 mM | 3 | 13.0 | 5.6 | 3.2 | 8 | 19 | 11 |
| Disuccinate | | | | | | | | | |
| Sodium Salt | | | | | | | | | |
| Lutein | Water | 3.0 mM | 3 | 61.7 | 4.0 | 2.3 | 58 | 66 | 8 |
| Disuccinate | | | | | | | | | |
| Sodium Salt | | | | | | | | | |
| Lutein | Water | 5.0 mM | 3 | 74.7 | 4.5 | 2.6 | 70 | 79 | 9 |
| Disuccinate | | | | | | | | | |
| Sodium Salt | | | | | | | | | |

TABLE II
Descriptive statistics of mean % inhibition of superoxide anion signal for aqueous and
ethanolic (40%) formulations of disodium diphosphate derivatives of natural source lutein
tested in the current study. Sample sizes of 3 were evaluated for each formulation, with
the exception of lutein diphosphate in water at 100 μM (0.1 mM) where N = 1. Mean %
inhibition of superoxide anion signal increased in a dose-dependent manner as the concentration
of lutein diphosphate was increased in the test assay. At 100 μM in water, no inhibition
of scavenging was seen. The molecular solutin in 40% EtOH (mean % inhibition = 18%)
was increased above background scavenging (5%) by the ethanolic vehicle, suggesting that
disaggregation increased scavenging at the concentration. Slightly increased scavenging
(on a molar basis) may have been obtained with the diphosphate derivative in comparison
to disuccinate derivative (see Table 1 and FIG. 8).

| | | | | Mean (% | | | | | |
|---|---|---|---|---|---|---|---|---|---|
| Sample | Solvent | Concentration | N | inhibition) | S.D. | SEM | Min | Max | Range |
| Lutein | 40% | 0.1 mM | 3 | 18.0 | 7.0 | 4.0 | 11 | 25 | 14 |
| Diphosphate | EtOH | | | | | | | | |
| Sodium Salt | | | | | | | | | |
| Lutein | Water | 0.1 mM | 1 | 0.0 | ND | ND | 0 | 0 | 0 |
| Diphosphate | | | | | | | | | |
| Sodium Salt | | | | | | | | | |
| Lutein | Water | 1.0 mM | 3 | 9.3 | 3.5 | 2.0 | 6 | 13 | 7 |
| Diphosphate | | | | | | | | | |
| Sodium Salt | | | | | | | | | |
| Lutein | Water | 3.0 mM | 3 | 72.3 | 3.1 | 1.8 | 69 | 75 | 6 |
| Diphosphate | | | | | | | | | |
| Sodium Salt | | | | | | | | | |
| Lutein | Water | 5.0 mM | 3 | 91.0 | 2.6 | 1.5 | 88 | 93 | 5 |
| Diphosphate | | | | | | | | | |
| Sodium Salt | | | | | | | | | |

3 were evaluated for each formulation, with the exception of lutein diphosphate in water at 100 μM (0.1 mM) where N=1. Mean % inhibition of superoxide anion signal increased in a dose-dependent manner as the concentration of lutein diphosphate was increased in the test assay. At 100 μM in water, no inhibition of scavenging was seen. The molecular solution in 40% EtOH (mean % inhibition=18%) was increased above background scavenging (5%) by the ethanolic vehicle, suggesting that disaggregation increased scavenging at that concentration. Slightly increased scavenging (on a molar basis) may have been obtained with the diphosphate derivative in comparison to disuccinate derivative (see Table 1 and FIG. 8).

In the current study, facile preparations of the disodium disuccinate and tetrasodium phosphate esters of natural source (RRR) lutein are described. These asymmetric C40 carotenoid derivatives exhibited aqueous dispersibility of 2.85 and 29.27 mg/mL, respectively. Evidence for both card-pack (H-type) and head-to-tail (J-type) supramolecular assembly was obtained with UV-Vis spectroscopy for the aqueous solutions of these compounds. Electronic paramagnetic spectroscopy of direct aqueous superoxide scavenging by these derivatives demonstrated nearly identical dose-dependent scavenging profiles, with slightly increased scavenging noted for the diphosphate derivative. In each case, scavenging in the millimolar range was observed. These results show that as parenteral soft drugs with aqueous radical scavenging activity, both compounds are useful in those clinical applications in which rapid and/or intravenous delivery is desired for the desired therapeutic effect(s).

The effects of COX selective and non-selective agents on rates of peroxidation was tested in various lipid preparations with polyunsaturated fatty acids, including DAPC (1,2-diarachidonoyl-sn-glycero-3-phosphocholine). These lipids along with cholesterol powder were obtained from Avanti Polar Lipids (Alabaster, Ala.) and characterized by HPLC. Astaxanthin (all-trans 3S,3′S; chiral purity >97%) was synthesized by Synchem, Inc. (Des Plaines, Ill.; patents pending) and supplied by Hawaii Biotech, Inc. (HBI). Other chemical reagents and all drugs were purchased from independent commercial sources, including Sigma (St. Louis, Mo.), Acros Organics (Morris Plains, N.J.), and Calbiochem (San Diego, Calif.). Depending on their solubility properties, drugs and sulfone compounds (methyl phenyl sulfone, dimethyl sulfone) in powder form were solubilized in organic solvents before adding to buffer at low volumes. Lipids were stored at −80° C. in HPLC-grade chloroform.

### LDL Isolation and Oxidation Analysis

The effects of the compounds on lipid peroxidation were tested in isolated human LDL. Human venous blood was collected into vacutainer tubes containing K₃EDTA after a 12-hour fast. Plasma was immediately separated by centrifugation at 3000 g for 25 minutes. LDL was obtained from plasma by ultracentrifugation using a procedure similar to that reported by Chung et al, 1986 using discontinuous KBr gradient. The purity of the freshly isolated LDL was assessed by SDS-PAGE using the procedure of Laemmli, 1970.

The extent of oxidation was measured for the LDL in the absence and presence of the drugs over a range of concentrations. Malondialdehyde (MDA), a product of lipid oxidation, was measured by the reaction of this aldehyde with thiobarbituric acid in an acid medium to form a stable chromogen, referred to as thiobarbituric-acid-reactive-substances (TBARS). Purified LDL (100 μg protein/ml) was incubated for 30 minutes with either vehicle or an NSAID at various concentrations followed by addition of 50 μM CuSO₄ at 37° C. Conjugated diene formation was measured by continuously monitoring the change in absorbance at 234 nm on a Beckman DU 640 spectrophotometer, as described by Esterbauer et al., 1985. Stock solutions were tested for iron contamination, which can contribute independently to lipid peroxidation and assay artifacts.

The ability of astaxanthin to block the pro-oxidant effects of rofecoxib was tested in lipid vesicles reconstituted from DAPC (10 mg/ml) in physiologic buffer (0.5 mM HEPES, 154 mM NaCl, pH=7.32). The lipid vesicles also contained cholesterol at a level that reproduced physiologic conditions (0.2 cholesterol to phospholipid mole ratio). The drug effects were tested in the vesicles following addition of astaxanthin, rofecoxib or the combination of these two agents at an identical concentration (250 nM). To measure lipid peroxide (LOOH) formation, we used an assay that measures the conversion of I₂ to I₃⁻ (triiodide). This reaction takes place in the presence of LOOH in a manner that can be measured photometrically at 365 nm, El-Saadani, 1989. This assay is sensitive to peroxide concentrations as low as 10 μM and has the further advantage in that it does not require the use of exogenous peroxide radical initiators. The lipid peroxidation reaction occurs gradually under normal atmospheric oxygen conditions in a shaking water bath (37° C.).

Determination of F₂-Isoprostanes by Gas Chromatography (GC) with Negative Chemical Ionization Mass Spectroscopy (NCI-MS)

F₂-Isoprostanes are derived principally from the formation of positioned peroxyl radical isomers of arachidonic acid, endocyclization to protaglandin G₂-like structures, and reduction to PGF₂-like compounds. Total levels of F₂-isoprostanes, in DAPC lipid vesicles prepared in the presence of vehicle or drug, were measured by GC-MS with negative chemical ionization as described by Walter et al., 2000. Peroxidation of lipids occurred over time in the absence of any exogenous initiators at 37° C. F₂-Isoprostane formation was also measured independently using mass spectroscopy in a blinded study at the Antioxidant Research Laboratory, Tufts University, Boston, Mass.

### Oxygen Radical Absorption Capacity (ORAC) of Human Plasma

The comparative effect of COX-2 selective agents on the antioxidant capacity of human plasma was assessed using the Oxygen Radical Absorption Capacity (ORAC) assay. This assay was carried out according to the method of Huang et al., 2002 with fresh plasma using a microplate fluorescence reader in 96 well format with the excitation and emission filters set at 485 and 530 nm respectively.

### Preparation of Lipid Vesicles for X-Ray Diffraction and Oxidation Analysis

The effects of selective and non-selective COX inhibitors on membrane molecular structure and rates of lipid peroxidation were assessed in multilamellar lipid vesicles (MLVs). MLVs were prepared in buffer (0.5 mmol/L HEPES and 154.0 mmol/L NaCl, pH, 7.2) by the method of Bangham, 1965. For x-ray diffraction analysis, the final phospholipid (POPC) concentration was 2.5 mg/ml and the mole ratio of cholesterol to phospholipid was 0.2:1. The mole ratio of drug to phospholipid was 1:10, resulting in a final concentration of <5%, by mass. Membrane samples were oriented for x-ray diffraction and analyzed, as previously described in detail.

### Small-Angle X-Ray Diffraction Analysis of Drug/Lipid Structure

Small-angle x-ray diffraction approaches were used to examine the effects of COX-2 inhibitors on the time-averaged molecular structure of lipids in vascular cell-like membranes. X-ray diffraction experiments were conducted by aligning the samples at grazing incidence with respect to a collimated x-ray source. Corrected diffraction orders obtained from samples in this study were analyzed using Fourier summation to yield one-dimensional electron density profiles (A versus electrons/Å³) of the membrane lipid bilayer.

### Statistical Analysis

Data are presented as mean ±S.D. The significance of differences between results from independent experimental conditions (conducted in triplicate) was tested using the two-tailed Student t-test. A value of p<0.05 was considered significant.

### Rofecoxib Increases the Susceptibility of Human LDL to Oxidative Modification: Comparison to Other COX-2 Inhibitors and NSAIDs

Minimally modified or oxidized LDL has an essential role in atherosclerotic plaque instability by contributing to mechanisms of endothelial dysfunction and inflammation. We evaluated the effects of rofecoxib on rates of lipid peroxidation in isolated human LDL and lipid vesicles enriched with polyunsaturated fatty acids (e.g., arachidonic acid). Lipid peroxidation in these various biological preparations was monitored and compared to other selective (rofecoxib, etoricoxib, celecoxib, valdecoxib), preferential (meloxicam) and non-selective (ibuprofen, naproxen, diclofenac) COX inhibitors under identical conditions. The activity of rofecoxib was also compared to sulfone analogs, including methyl phenyl sulfone and dimethyl sulfone.

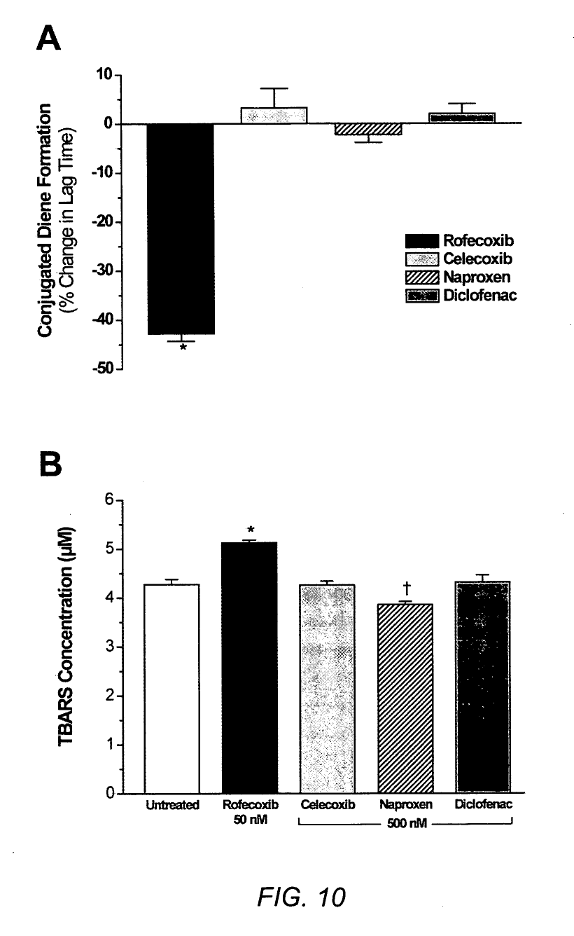

Following incubation with human LDL, rofecoxib significantly (p<0.001) decreased the lag time for human LDL conjugated diene formation by 42.8±1.5% at 100 nM (FIG. 10A). This pronounced effect on the rate of conjugated diene formation indicates that rofecoxib has potent pro-oxidant activity, as evidenced by depleted LDL antioxidant capacity. In addition to measuring conjugated diene formation, we also measured the formation of reactive aldehydes, especially malondialdehyde (MDA). Consistent with its effect on conjugated diene formation, rofecoxib and etoricoxib also caused marked increases in MDA levels. The comparative effects of these agents on MDA formation from human LDL (measured as TBARS) are reported in FIG. 10B. Compared to celecoxib, ibuprofen, naproxen and diclofenac, only rofecoxib caused a significant increase in MDA levels, even at a concentration (50 nM) that was 10-fold lower (50 nM) that that used for comparison drugs. Pro-oxidant changes in conjugated diene formation or MDA levels were not observed following treatment with other COX selective or non-selective inhibitors under identical conditions. Additionally, other sulfone-containing compounds (methyl phenyl sulfone, dimethyl sulfone) had no effect on LDL oxidation. As compared to vehicle treated LDL samples (MDA level of 3.23±0.28 μM), there were no significant changes in LDL oxidation for samples treated with either dimethyl sulfone (3.32±0.19 μM, p=0.9) or methyl phenyl sulfone (3.25±0.13 μM, p=0.7) at a concentration of 1 μM.

Effect of COX-2 Inhibitors and NSAIDs on Isoprostane Formation from Membrane Phospholipids

Isoprostanes are prostaglandin isomers that can be generated non-enzymatically by free radical modification of arachidonic acid associated with phospholipid in LDL and cellular membranes. F₂-isoprostanes have been specifically identified in atherosclerotic plaques and oxidized LDL levels have been correlated with severity of acute coronary syndromes and plaque instability. We tested the effects of these agents on peroxidation of lipid vesicles containing arachidonic acid (DAPC), the substrate for non-enzymatic formation of isoprostanes. Using mass spectroscopy, we observed pronounced differences in isoprostane generation among the COX-2 inhibitors. Levels of isoprostanes were shown to increase from 140±25 ng/100 μl (mean ±S.D.) in vehicle-treated samples to 190±18 ng/100 μl (p<0.0025) and 224±22 ng/100 μl (p<0.0001) in the presence of 100 nM rofecoxib and etoricoxib, respectively (FIG. 1A). By contrast, celecoxib had no significant effect on peroxidation of arachidonic acid-enriched vesicles, even at higher concentrations (data not shown).

### Effect of Rofecoxib and Celecoxib on Oxygen Radical Antioxidant Capacity (ORAC) of Human Plasma

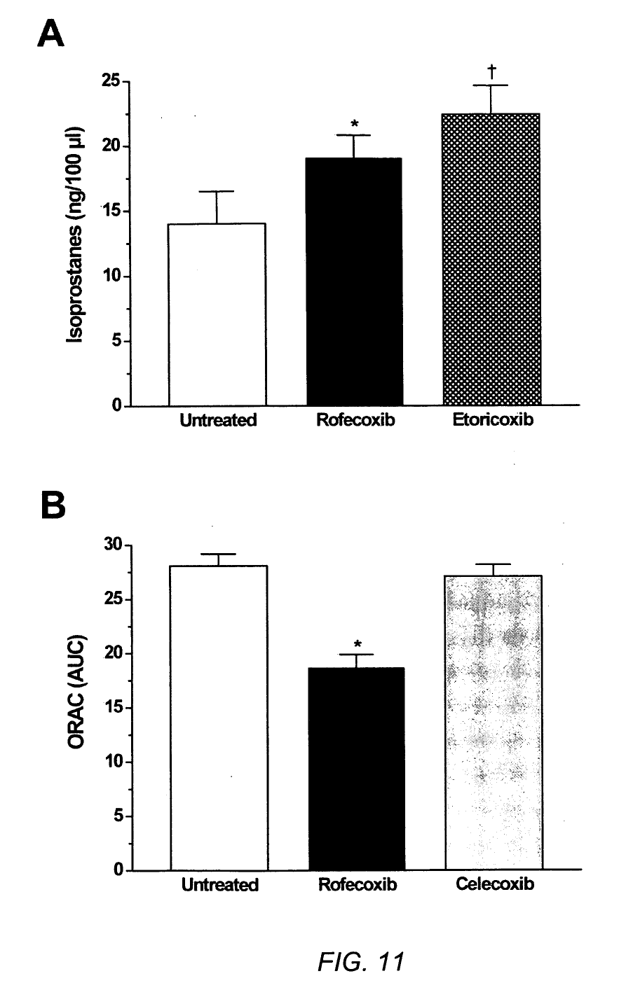

The comparative effects of rofecoxib and celecoxib on the antioxidant capacity of human plasma were assessed using the Oxygen Radical Absorption Capacity or ORAC assay. The area under the curve (AUC) from the ORAC analysis is reported in FIG. 11B. Consistent with a pro-oxidant effect, rofecoxib significantly (p<0.001) reduced the ORAC value by 34% (28.1±1.2 in vehicle-treated samples to 18.6±1.3) at 1.0 μM. In parallel experiments, celecoxib did not significantly change this value, even at the highest concentration tested (10.0 μM), in which the ORAC value was 28.5±0.1.

As reported in the Physicians Desk Reference (2003), the maximum plasma concentration (Cₘₐₓ) for celecoxib at an approved 200 mg dose is 1.85 μM (705 ng/ml). In the case of rofecoxib, an approved dose of 25 mg results in a plasma concentration of 658 nM (207 ng/ml). In the case of astaxanthin, a 100 mg oral ‘racemic’ dose results in a maximum plasma concentration of 2.18 μM (1.3 mg/L; Østerlie et al. 2000). Thus, the effects we are reporting with these drugs and astaxanthin at nanomolar to low micromolar concentrations are pharmacologically relevant.

Inhibition of Lipid Peroxidation with Rofecoxib by Astaxanthin

Astaxanthin (3,3′-dihydroxy-β,β′-carotene-4,4′-dione) is a carotenoid with significant antioxidant activity, even as compared to other compounds in this class. This highly lipophilic molecule reduces oxidative damage to biological lipids by quenching singlet oxygen and scavenging free radicals. The chemical basis for its activity is a 40-carbon polyene chromophore terminated by cyclic end groups with oxygen-containing polar substituents. In addition to their role in electron stabilization during the scavenging process, the polar terminal groups allow for a preferred membrane orientation that facilitates its scavenging properties. It has no pro-vitamin A activity in mammals (Jyonouchi et al. 2000).

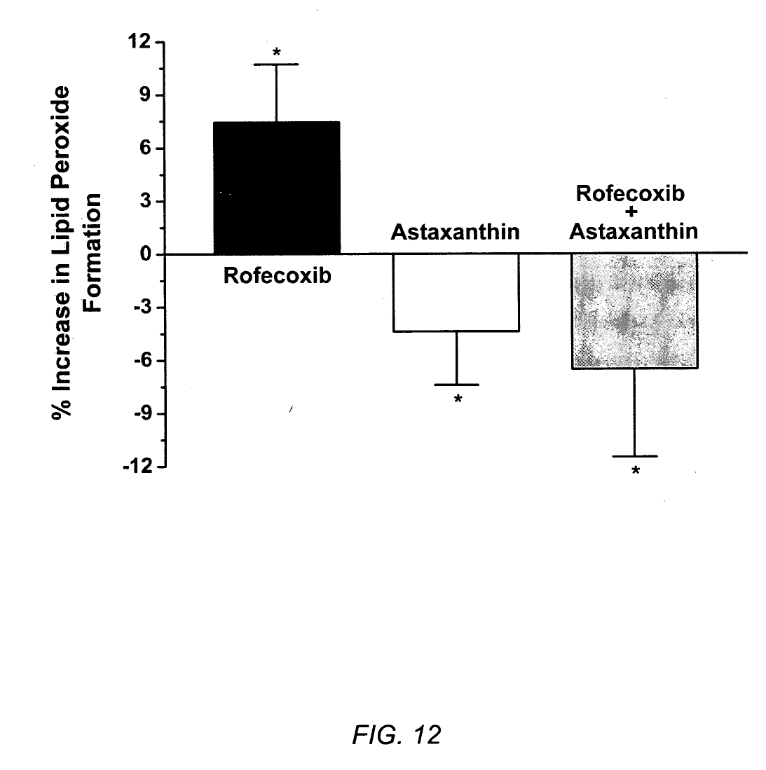

As shown in FIG. 12, the addition of rofecoxib alone at 250 nM separately caused a significant 7.4% increase (p<0.01 vs control) in lipid peroxide levels in vesicles enriched with PUFAs (arachidonic acid), consistent with our measurements of isoprostane formation. The effect of rofecoxib on lipid peroxidation was assessed after a 48 hour incubation period at 37° C. By contrast, addition of astaxanthin alone at this same concentration produced an opposite, antioxidant effect as evidenced by a 4.4% decrease (p<0.01 vs control) in lipid peroxidation. Remarkably, astaxanthin was able to completely inhibit the adverse effects of rofecoxib on lipid peroxidation when added together with rofecoxib at an equimolar concentration (decrease in lipid peroxidation of 6.5%; p<0.01 vs control).

### Effect of COX-2 Inhibitors on Lipid Structure

To understand the physico-chemical basis for differences in the effects of COX-2 inhibitors on the susceptibility of LDL and membrane lipids to oxidative modification, small-angle x-ray diffraction approaches were used to measure the effects of these agents on membrane structure. The addition of sulfone and sulfonamide COX-2 inhibitors at a low concentration (<5% by mass) produced separate changes in the molecular structure of phospholipid molecules, consistent with their distinct locations in the membrane bilayer. The addition of rofecoxib produced a discrete increase in electron density limited to the phospholipid headgroup region, 22-28 Å from the center of the membrane. This increase is attributed to the equilibrium location of the drug in the headgroup and hydrated surface of the membrane. Additionally, the presence of rofecoxib produced a decrease in electron density in the hydrocarbon core, 0-10 Å from the membrane center, a change similar to that associated with thermal heating or oxidative damage to the membrane. Thus, the pro-oxidant activity of rofecoxib may be related, in part, to physico-chemical changes in lipid structure, as opposed to electron transfer mechanisms associated with the sulfone group.

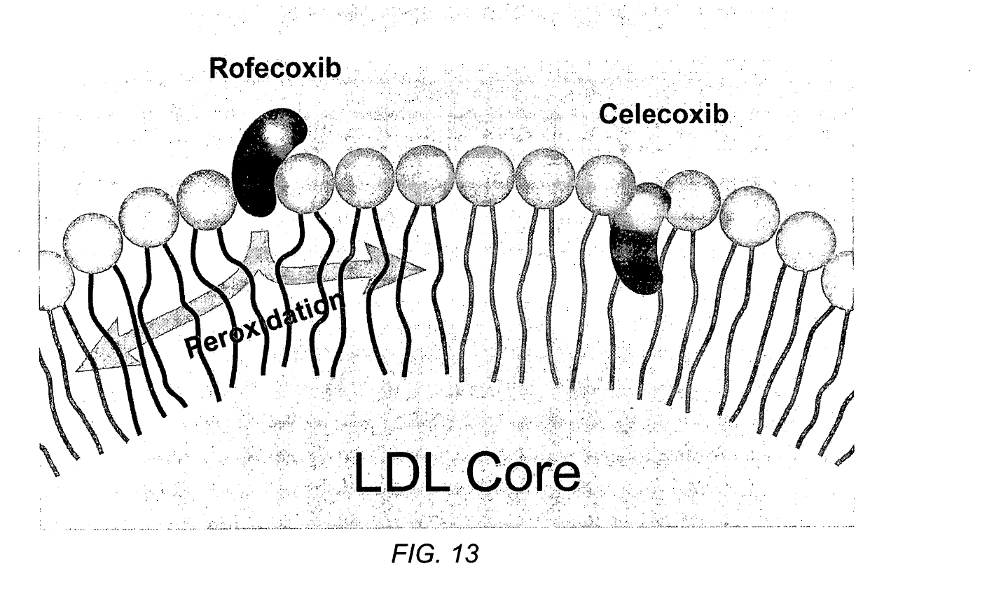

The addition of celecoxib to the phospholipid bilayer produced an increase in electron density associated with the upper hydrocarbon core of the membrane, 5-20 Å from the center of the membrane. Thus, a portion of the lipophilic celecoxib molecule being associated with the phospholipid acyl chains, adjacent to the headgroup region. The location of rofecoxib in the phospholipid headgroup region caused disordering of the phospholipid acyl chain. The alteration in the intermolecular packing of the lipid molecules may facilitate the propagation of free radicals. By contrast, celecoxib had a well-defined location in the membrane hydrocarbon core; a position consistent with its highly lipophilic properties. The equilibrium position of the celecoxib molecule did not cause a disordering in the lipid molecules. The covalent structure of celecoxib includes a trifluoromethly group, a substituent associated with hydrophobic properties that may underlie the membrane location and high volume of distribution for celecoxib (400 L). These differences in the molecular membrane interactions of the COX-2 inhibitors may contribute to their distinct physico-chemical effects on lipid peroxidation (FIG. 13).

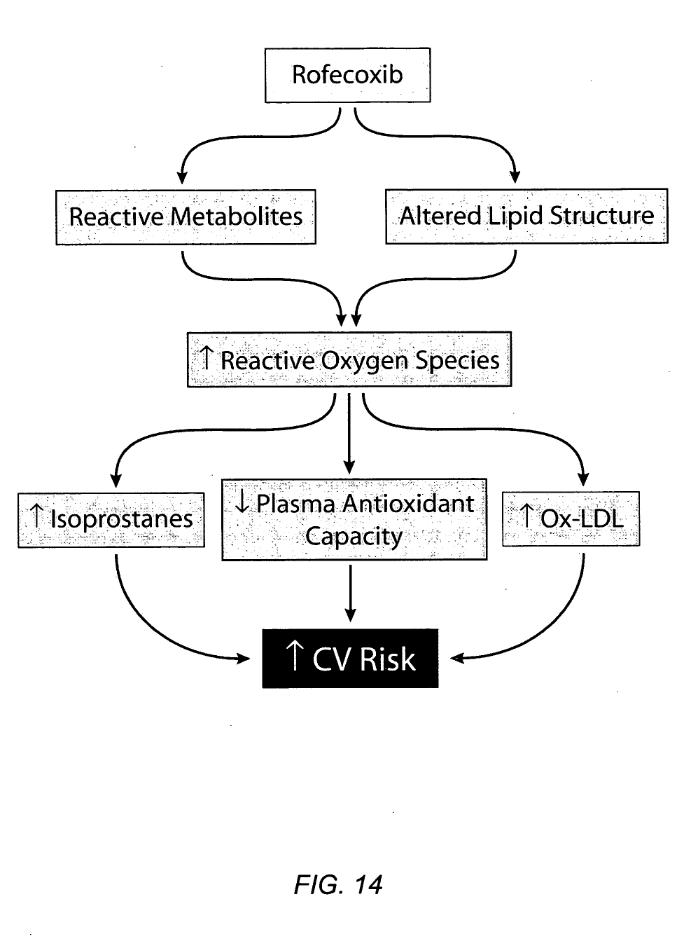

A mechanistic basis for cardiotoxicity with rofecoxib can be attributed to its distinct chemical properties and pro-oxidant activity (FIG. 14). Rofecoxib readily forms a highly reactive maleic anhydride derivative capable of reacting with various biological targets, including PUFAs, to form atherogenic reactive aldehydes and isoprostanes. This has been experimentally demonstrated with rofecoxib at low concentrations in isolated samples of human LDL and biological lipids. We have also now demonstrated a pharmacologic approach to block the pro-oxidant effects of rofecoxib using a highly lipophilic chain-breaking antioxidant, astaxanthin. Collectively, these findings indicate that rofecoxib is cardiotoxic due to inherent chemical properties that are unrelated to COX-2 inhibition.

### Overview of Methods

In this study, the comparative antioxidant activity of hydrocarbon-carotenoids (β-carotene and lycopene) and xanthophylls (astaxanthin, zeaxanthin, and lutein) were compared in a DLPC model membrane system. Antioxidant activities of the carotenoids were determined by incubating each carotenoid with the DLPC multilamellar vesicles at 1 and 10 μM. The levels of lipid peroxide formation were measured photometrically at specific time points.

### Rationale for Methods

The measurement of antioxidant activity is highly dependent on the experimental system used, and one should be cautious in comparing results of separate studies, or of extending the conclusions of a given study beyond its experimental limits. Almost all antioxidant assay methods developed for lipid systems are based on inhibition of free radicals induced by strong radical initiators added exogenously to the system. It has been reported that the outcomes of the assays are affected by the type and amount of initiator used in the system. To avoid the use of strong peroxide radical initiators employed by most antioxidant capacity assays, we chose the method developed by El-Saadani (13) which occurs under normal atmospheric oxygen conditions without addition of exogenous initiators. This method has several advantages over the conventional assays, and makes the present model study more valid from the physiological point of view.

1) Intrinsic antioxidant properties of carotenoids can be more accurately measured and compared without intervention of radical initiators;
2) Certain carotenoids incorporated into lipid bilayer membranes are known to limit the penetration of oxygen or small reactive oxygen species into membranes (14) by changing mechanical properties of membranes (15). Certain free radical initiators could interfere with such an important antioxidant mechanism;
3) Since the oxidation reaction in vivo occurs without added radical initiators, this method simulates physiological condition more effectively.

Woodall et al. (1997) reported that the antioxidant capacities of various carotenoids in liposomes are quite different from those in free solution. This suggests that the antioxidant properties of carotenoids are determined by how these molecules are incorporated and interact with membranes. Consistent with this finding, one study (Subczynski, 1991) indicated that the protection of biomembranes against oxidative damage by carotenoids can be explained by the modification of the physical properties of membranes caused by the incorporation of carotenoids.

We hypothesize that small differences in carotenoid structure have a significant effect on the way that these molecules function in membranes, including their antioxidant activity. In this study, the antioxidant capacities of 5 carotenoids were investigated and correlated with their effects on membrane phospholipid structure by using the small angle x-ray diffraction approaches. The effects of the carotenoids on membrane structure and rates of lipid peroxidation will be tested in phospholipid vesicles under normal and atherosclerotic-like conditions characterized by cholesterol enrichment (as produced by an increase in cholesterol:phospholipid ratio or C/P).

### EXPERIMENTAL PROCEDURES

### Example 1

The experimental procedures disclosed herein are substantially similar to those disclosed in Walter et al., 2004, which is hereby incorporated by reference.

Materials

The following reagents were obtained from the indicated source and were prepared as set forth below.
1. 1,2-Dilinoleaoyl-sn-Glycero-3-Phosphocholine (DLPC): Avanti Polar-Lipids, Inc., Lot # 182PC-172 g, FW=830.14, concentration 10 mg/ml in chloroform. DLPC was stored at −80° C.
2. 1-Palmitoyl-2-Oleoyl-sn-Glycero-3-Phosphocholine (POPC): Avanti Polar-Lipids, Inc., Lot # 160-181PC-148, FW=760.1, concentration 25 mg/ml in chloroform. POPC was stored at −80° C.
3. Cholesterol: Avanti Polar-Lipids, Inc., Lot # CH-50, FW=386.66, concentration 10 mg/ml in chloroform Lipid is stored at −20° C.
4. Diffraction Buffer: 0.5 mM HEPES, 154 mM NaCl, pH=7.32.
5. Astaxanthin (all-trans-3S,3′S): Albany Molecular Research, Inc., Lot # MY966-Z, FW=596.84, concentration 500 μM in chloroform. Drug was stored at −20° C.
6. β-Carotene (all-trans): Sigma-Aldrich, Lot # 094K7038, FW=536.86, concentration 500 μM in chloroform. Drug was stored at −20° C.
7. Lutein (all-trans-3R,3′R,6′R; RRR-lutein): Chromadex, Inc., Lot # 12453-056, FW=568.88, concentration 500 μM in chloroform. Drug was stored at −80° C.
8. Zeaxanthin: CaroteNature GmbH, Lot # 0119, FW=568.88, concentration 500 μM in chloroform. Drug was stored at −20° C.
9. Lycopene: Chromadex, Inc., Lot # 12550-318, FW=536.89, concentration 500 μM in chloroform. Drug was stored at −20° C.
10. Rofecoxib (Vioxx): Merck & Co., Inc., MW=314.36 g/mol, concentration 50 μM in methanol. Drug is stored at −20° C. until use.
11. CHOD Stock Reagent
   a. Materials
      (1) Potassium phosphate, monobasic (KH₂PO₄). FW=136.08. Reagent concentration=0.2 M.
      (2) Potassium iodide (KI). FW=166.0. Reagent concentration=0.12 M.
      (3) Sodium azide (NaN₃). FW=65.01. Reagent concentration=0.15 mM.
      (4) Ammonium molybdate (tetrahydrate). FW=1235.9. Reagent concentration=10 μM.
      (5) Benzalkonium chloride. Reagent concentration=0.1 g/L.
   b. The following steps were performed to make CHOD stock reagent
      (1) Add approximately 300 ml double deionized water (ddH₂O) to a 1 L beaker and begin stirring on a magnetic stir plate.
      (2) Add 27.22 g potassium phosphate monobasic to dissolution.
      (3) Add 19.92 g potassium iodide to dissolution.
      (4) Transfer 200 μl of 750 mM sodium azide stock to the solution.
      (5) Transfer 400 μl of 25 mM ammonium molybdate stock to the solution.
      (6) In a separate (50 ml) beaker, weigh out 0.1 g benzalkonium chloride. [NOTE: May need to heat briefly on hot plate to return to liquid state.]
      (7) Add 20-30 ml ddi water to small beaker and agitate benzalkonium chloride with spatula until completely dissolved.
      (8) Transfer benzalkonium solution to CHOD reagent. [NOTE: Adding the benzalkonium solution to the CHOD reagent will result in immediate turbidity.]
      (9) Bring total volume up to ˜900 ml (but do not exceed) by adding double deionized water.
      (10) Cover with foil and stir for several hours until turbidity decreases significantly.
      (11) Transfer contents of beaker to a IL volumetric flask and add double deionized to 1 L. Insert stir bar and return to stir plate for ˜1 hour.
      (12) Transfer CHOD-Color reagent to amber bottle(s) and store at 4° C. until use.
12. CHOD Active Reagent [NOTE] Active reagent must be made freshly made right before use.
   a. Materials
      (1) Triton-X. Reagent concentration=0.2%
      (2) Ethylenediaminetetraacetic acid (EDTA; C₁₀H₁₄N₂O₈Na₂×2H₂O). MW=372.2. Reagent concentration=24 μM
      (3) Butylated hydroxytoluene (BHT; C₁₅H₂₄O). FW=220.4. Reagent concentration=20 μM
   b. Methods
      (1) Added 200 ml of CHOD stock reagent to 250 ml flask.
      (2) Added 960 μl of EDTA stock (5 mM) to achieve 24 μM.
      (3) Added 114 μl of BHT stock (35 mM) to achieve 20 μM.
      (4) Added 400 μl of Triton-X to achieve 0.2%.
      (5) Stir mixture for ˜1 hour at a slow speed setting.
      (6) Filter final active CHOD reagent using Whatman #1 filter paper.

### Example 2

### 1. Experimental Outline

TABLE III
Sample Design Summary

| Sample | | DLPC | Cholesterol | Carotenoid |
|---|---|---|---|---|
| # | Description | (μl) | (μl) | (μl) |
| 1-6 | DLPC + Cholesterol | 100 | 10 | — |
| | (Control) | | | |
| 7-12 | DLPC + Cholesterol + | 100 | 10 | 2 |
| | 1 μM Astaxanthin | | | |
| 13-18 | DLPC + Cholesterol + | 100 | 10 | 20 |
| | 10 μM Astaxanthin | | | |
| 19-24 | DLPC + Cholesterol + | 100 | 10 | 2 |
| | 1 μM β-Carotene | | | |
| 25-30 | DLPC + Cholesterol + | 100 | 10 | 20 |
| | 10 μM β-Carotene | | | |
| 31-36 | DLPC + Cholesterol + | 100 | 10 | 2 |
| | 1 μM Lutein | | | |
| 37-42 | DLPC + Cholesterol + | 100 | 10 | 20 |
| | 10 μM Lutein | | | |
| 43-48 | DLPC + Cholesterol + | 100 | 10 | 2 |
| | 1 μM Zeaxanthin | | | |
| 49-54 | DLPC + Cholesterol + | 100 | 10 | 20 |
| | 10 μM Zeaxanthin | | | |
| 55-60 | DLPC + Cholesterol + | 100 | 10 | 2 |
| | 1 μM Lycopene | | | |
| 61-66 | DLPC + Cholesterol + | 100 | 10 | 20 |
| | 10 μM Lycopene | | | |

### Formation of Multilamellar Vesicles (MLVs)

1. Samples were prepared in a glass test tube as described in the table above. This gives a cholesterol/DAPC mole ratio of 0.2.
2. All samples were dried down under a steady stream of nitrogen gas while vortexing.
3. Samples were placed in a vacuum desiccator for 2 hours to remove residual solvent and protected from light.
4. After samples were removed from the vacuum desiccator, they were immediately resuspended in 1 ml of diffraction buffer, warmed to room temperature.
5. Sample tubes were vortexed for 3 min to form MLVs.

### Example 3

### Measurement of Lipid Peroxide Levels

This assay is based on the principle of the oxidative capacity of lipid peroxides to convert 12 to 13-(triiodide), which can be measured photometrically at 365 μm (El-Saadani et al., 1989). This assay is sensitive to peroxide concentration as low as 10 μM.
1. Samples were placed in a water bath set at 37° C. Samples remained in the water bath throughout the experiment except for the temporary removal for time point extractions.
2. To avoid reading >1 Au, aliquots of a sample varying in volume were extracted for each time point as described in Table IV below.

TABLE IV

| Time | Sample | | | |
|---|---|---|---|---|
| point | Volume | CHOD (active) | Diffraction Buffer | Total Volume |
| (hr) | (μl) | Volume (μl) | Volume (μl) | (μl) |
| 24 | 50 | 1,000 | 50 | 1100 |
| 48 | 80 | 1,000 | 20 | 1100 |

3. After combining sample time point aliquots with CHOD active reagent and the diffraction buffer as described in the table above, test samples were incubated about 6 hours in darkness at ambient temperature.
4. The absorbance (A₃₆₅ nm) of the samples was measured using a spectrophotometer.
5. Concentrations of lipid peroxides were calculated by the use of the molar absorptivity of I₃⁻ measured at 365 mm (ε=2.46±0.25×10⁴ M⁻¹ cm⁻¹). This value was determined by adding known concentrations of I₂ to the CHOD reagent. A stoichiometric relationship (slope=1.02) was observed between the amount of organic peroxides assayed and the concentration of I₃⁻ produced.

### Example 4

RESULTS The results contained using the methods described above are summarized in Tables V and VI below.

TABLE V
Effects of Carotenoids at Two Different Concentrations on Lipid Peroxidation (24 Hour)
Lipid Peroxide Levels (μM)

| | Astaxanthin | | β-Carotene | | Lutein | | Zeaxanthin | | Lycopene | |
|---|---|---|---|---|---|---|---|---|---|---|
| | (μM) | | (μM) | | (μM) | | (μM) | | (μM) | |
| Control | 1 | 10 | 1 | 10 | 1 | 10 | 1 | 10 | 1 | 10 |
| 218 ± 20 | 203 ± 17 | 169 ± 7* | 272 ± 10* | 369 ± 17* | 252 ± 11^{†} | 220 ± 8 | 241 ± 12^{‡} | 217 ± 7 | 239 ± 2^{‡} | 405 ± 32* |
| | −6.9%^{a} | −22.5% | +24.8%^{a} | +69.3%^{a} | +15.6%^{a} | +0.9%^{a} | +10.6%^{a} | −0.5%^{a} | +9.6%^{a} | +85.8%^{a} |

Values are mean ± S.D. (n = 5~6),
*P < 0.001 vs control;
^{†}P < 0.01 vs control;
^{‡}P < 0.05 vs control
^{a}% increase (+) or decrease (−) compared to control

TABLE VI
Effects of Carotenoids at Two Different Concentrations on Lipid Peroxidation (48 Hour)
Lipid Peroxide Levels (μM)

| | Astaxanthin | | β-Carotene | | Lutein | | Zeaxanthin | | Lycopene | |
|---|---|---|---|---|---|---|---|---|---|---|
| | (μM) | | (μM) | | (μM) | | (μM) | | (vM) | |
| Control | 1 | 10 | 1 | 10 | 1 | 10 | 1 | 10 | 1 | 10 |
| 572 ± 76 | 500 ± 57 | 339 ± 42* | 803 ± 99^{†} | 1068 ± 82* | 649 ± 73 | 673 ± 69^{‡} | 605 ± 26 | 690 ± 24^{†} | 647 ± 61 | 1253 ± 55* |
| | −12.6%^{a} | −40.7%^{a} | +40.4%^{a} | +86.7%^{a} | +13.5%^{a} | +17.7%^{a} | +5.8%^{a} | +20.6%^{a} | +13.1%^{a} | +119.1%^{a} |

Values are mean ± S.D. (n = 5~6),
*P < 0.001 vs control;
^{†}P < 0.01 vs control;
^{‡}P < 0.05 vs control
^{a}% increase (+) or decrease (−) compared to control

### Example 5

### Small Angle X-Ray Diffraction

### Sample Design Summary

| | | | | Carotenoids (μl) |
|---|---|---|---|---|
| | | | | Astaxanthin/ |
| | | | | β-carotene/ |
| | POPC | Cholesterol (μl)* | | Zeaxanthin/ |
| Sample Description | (μl) | C/P 0.2 | C/P 0.6 | Lutein/Lycopene |
| POPC + Cholesterol | 40.0 | 10.2 | 30.6 | — |
| (Control) | | | | |
| POPC + Cholesterol + | 40.0 | 10.2 | 30.6 | 17.6 |
| Astaxanthin | | | | |
| POPC + Cholesterol + | 40.0 | 10.2 | 30.6 | 17.6 |
| β-Carotene | | | | |
| POPC + Cholesterol + | 40.0 | 10.2 | 30.6 | 17.6 |
| Zeaxanthin | | | | |
| POPC + Cholesterol + | 40.0 | 10.2 | 30.6 | 17.6 |
| Lutein | | | | |
| POPC + Cholesterol + | 40.0 | 10.2 | 30.6 | 17.6 |
| Lycopene | | | | |

*Two sets of samples were constructed at the C/P (cholesterol/phospholipid) ratios of 0.2 and 0.6.

### Formation of Multilamellar Vesicles (MLVs)

1. Samples were prepared in glass test tubes as described in the table above. This gives a mole ratio of carotenoids/POPC 1/15. Equal volumes of vehicle (17.6 μl chloroform) were added to control samples.
2. All samples were dried under a steady stream of nitrogen gas while vortexing.
3. All samples were then placed in a vacuum desiccator for 1 hour to remove any residual solvent.
4. Multi-lamellar vesicles (MLVs) were prepared by the method developed by Bangham and Standish (1965). After samples were removed from the vacuum desiccator, they were immediately resuspended in 400 μl of diffraction buffer warmed to room temperature and vortexed for 3 minutes to form MLVs.
5. Aliquots of MLVs suspension (100 μl, 250 μg POPC equivalent) were transferred from each sample tube to respective Lucite® sedimentation cells (n=3). Each sedimentation cell consists of a solid base and a hollow cylindrical top between which is fastened a thin aluminum foil substrate, positioned to collect the membrane sample pellet upon centrifugation.
6. Membrane samples were centrifuged in a Sorvall AH-629 swinging bucket ultracentrifuge rotor (Dupont Corp., Wilmington, Del.) at 35,000×g for 1 hour at 5° C.
7. Following pellet collection, sample supernatants were aspirated followed by disassembly of the sedimentation cells; aluminum foil substrates, supporting the membrane pellets, were removed and mounted onto curved glass supports.
8. Samples were placed in hermetically sealed containers in which temperature and relative humidity were regulated prior to and during small angle x-ray diffraction analysis. In this experiment, all samples were examined at 20° C. and 87% relative humidity which was established using a saturated solution of potassium sodium tartrate (C₄H₄O₆ NaK.4H₂O). Then the same samples at a C/P ratio of 0.2 were equilibrated at 20° C., 74% relative humidity and subjected to x-ray diffraction analysis. The relative humidity of 74% was established using a saturated solution of L(+) tartaric acid (K₂C₄H₄O₆.½H₂O).

### Small Angle X-Ray Diffraction

1. The oriented membrane samples were aligned at grazing incidence with respect to a collimated, monochromatic x-ray beam (CuKα radiation, λ=1.54 Å) produced by a Rigaku Rotaflex RU-200, high-brilliance rotating anode microfocus generator (Rigaku USA, Danvers, Mass.). The fixed geometry beamline utilized a single Franks mirror providing nickel-filtered radiation (Kα₁ and Kα₂ unresolved) at the detection plane.
2. Diffraction data were collected on a one-dimensional, position-sensitive electronic detector (Innovative Technologies, Newburyport, Mass.), calibrated using cholesterol monohydrate crystals.
3. The sample-to-detector distance for this experiment was set to 150 mm.
4. Each individual diffraction peak used for x-ray diffraction analysis was background-corrected using a linear subtraction routine that averaged the noise. The lamellar intensity functions from oriented membrane samples were corrected by a factor of s=2 sin θ/λ, the Lorentz correction; phases were assigned based on previous swelling analyses of POPC membranes. Microcal Origin 7.0 software (OriginLab Corp., Northampton, Mass.) was used for performing these mathematical routines as well as for related analyses.
5. The unit cell periodicity, or d space, of the membrane lipid bilayer is the measured distance from the center of one lipid bilayer to the next, including surface hydration. The d spaces for the membrane multibilayer samples were calculated using Bragg's Law:

hλ=2d sin θ
in which h is the diffraction order number, λ is the wavelength of the x-ray radiation (1.54 Å), d is the membrane lipid bilayer unit cell periodicity, and θ is the Bragg angle equal to one-half the angle between the incident beam and scattered beam.
   6. Fourier transformation of the diffraction data yielded the electron density distributions intrinsic to the membrane samples. Each individual diffraction peak used for x-ray diffraction analysis was background-corrected using a linear subtraction routine that averaged the noise. The lamellar intensity functions from oriented membrane samples were corrected by a factor of s=2 sin θ/λ, the Lorentz correction; phases were assigned based on previous swelling analyses of POPC membranes. Microcal Origin 7.0 software (OriginLab Corp., Northampton, Mass.) was used for performing these mathematical routines as well as for other related analyses.

### Example 6

### Peroxidation Study

### Sample Design Summary

| | | | | Carotenoids (μl)) |
|---|---|---|---|---|
| | | | | Astaxanthin/ |
| | | | | β carotene/Lutein/ |
| Sample | Sample | DLPC | Cholesterol | Zeaxanthin/ |
| # | Description | (μl) | (μl) | Lycopene |
| 1-6 | DLPC + | 40 | 30 | — |
| | Cholesterol | | | |
| | (Control) | | | |
| 7-12 | DLPC + | 40 | 30 | 20 |
| | Cholesterol | | | |
| | Astaxanthin | | | |
| 13-18 | DLPC + | 40 | 30 | 20 |
| | Cholesterol + | | | |
| | β-Carotene | | | |
| 19-24 | DLPC + | 40 | 30 | 20 |
| | Cholesterol + | | | |
| | Lutein | | | |
| 25-30 | DLPC + | 40 | 30 | 20 |
| | Cholesterol + | | | |
| | Zeaxanthin | | | |
| 31-36 | DLPC + | 40 | 30 | 20 |
| | Cholesterol + | | | |
| | Lycopene | | | |

### Formation of Multilamellar Vesicles (MLVs)

1. Samples were prepared in a glass test tube as described in the table above. This gives a cholesterol/DLPC mole ratio of 0.6 and a final concentration of 10 μM for the carotenoids. Equal volumes of vehicle (20 μl chloroform) were added to control samples.
2. All samples were dried down under a steady stream of nitrogen gas while vortexing.
3. Samples were placed in a vacuum desiccator for 2 hours to remove residual solvent and protected from light.
4. Multi-lamellar vesicles (MLVs) were prepared by the method developed by Bangham and Standish (1965). After samples were removed from the vacuum desiccator, they were immediately resuspended in 1 ml of diffraction buffer warmed to room temperature and vortexed for 3 minutes to form MLVs.

### Measurement of Lipid Peroxide Levels

This assay is based on the principle of the oxidative capacity of lipid peroxides to convert I₂ to I₃⁻ (triiodide), which can be measured photometrically at 365 nm (el-Saadani et al., 1989). This assay is sensitive to peroxide concentrations as low as 10 μM.

1. Samples were placed in a water bath set at 37° C. throughout the experiment.
2. Aliquots of a sample (20 μl) were extracted at a 48 hour time point as described in the table below.

| Time | Sample | | | |
|---|---|---|---|---|
| point | Volume | CHOD (active) | Diffraction Buffer | Total Volume |
| (Hr) | (μl) | Volume (μl) | Volume (μl) | (μl) |
| 48 | 20 | 1,000 | 80 | 1100 |

3. After combining the samples with CHOD active reagent and the diffraction buffer, test samples were incubated for about 6 hours in darkness at ambient temperature.
4. The absorbance of a sample was measured photometrically at 365 nm.

Concentrations of lipid peroxides were calculated by the use of the molar absorptivity of I₃⁻ measured at 365 nm (ε=2.46±0.25×10⁴M⁻¹ cm⁻¹). This value was determined by adding known concentrations of 12 to the CHOD reagent. A stoichiometric relationship (slope=1.02) was observed between the amount of organic peroxides assayed and the concentration of I₃⁻ produced.

### Results

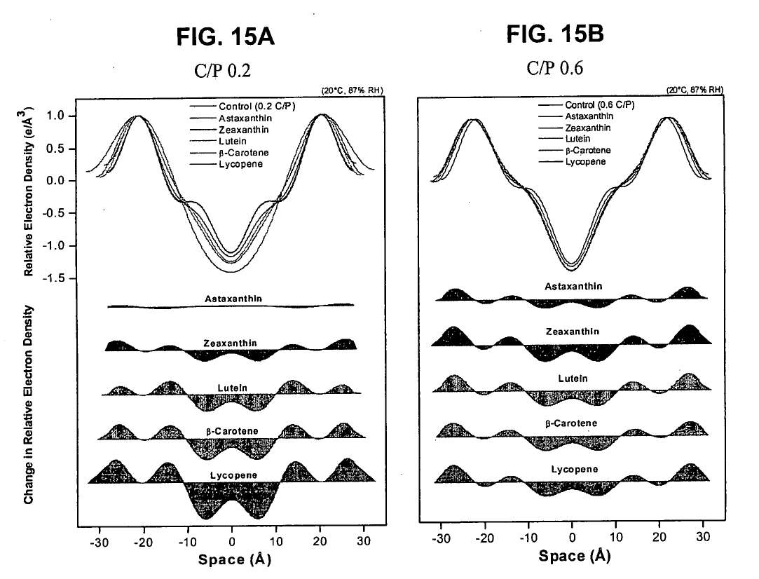

FIG. 15 shows representative membrane electron density profiles that were generated from the small angle x-ray diffraction data. To understand the effects of the carotenoids on membrane structure, the electron density profiles were superimposed on the same scale. With the exception of astaxanthin, all of the carotenoids alter membrane structure, but to different extents at a C/P of 0.2 (FIG. 15A).

Of particular interest, the carotenoids altered the electron density associated with the membrane hydrocarbon core over a broad range (±10 Å from the center of the membrane). The changes indicate that the compounds are disrupting the intermolecular packing of the phospholipids acyl chains near the membrane center. The greatest disordering effect was observed with lycopene, followed by β-carotene, lutein, and zeaxanthin (FIG. 15A). In addition to a decrease in electron density in the acyl chain regions, there was an increase in membrane width, as evidenced by an increase in d space values (Table 1).

TABLE 1

d Space (Å)

| Sample ID | 87% RH | 74% RH |
|---|---|---|
| Control | 55 | 55 |
| Astaxanthin | 57 | 55 |
| β-Carotene | 61 | 57 |
| Zeaxanthin | 58 | 57 |
| Lutein | 60 | 57 |
| Lycopene | 65 | 59 |

Changes in membrane structure were evaluated under conditions of high hydrostatic pressure. This condition would be expected to order the membrane lipid environment, independently of the effects of the carotenoids. Indeed, decreasing the relative humidity to 74% from 87% effectively blocked the disordering effects of carotenoids and reduced changes in lipid width (Table 1). This observation supports the idea that carotenoids exert their effects on membranes via a biophysical mechanism. If the changes are biochemical in nature, such as cleaved acyl chains, such a physical change would not be able to demonstrate this reversibility.

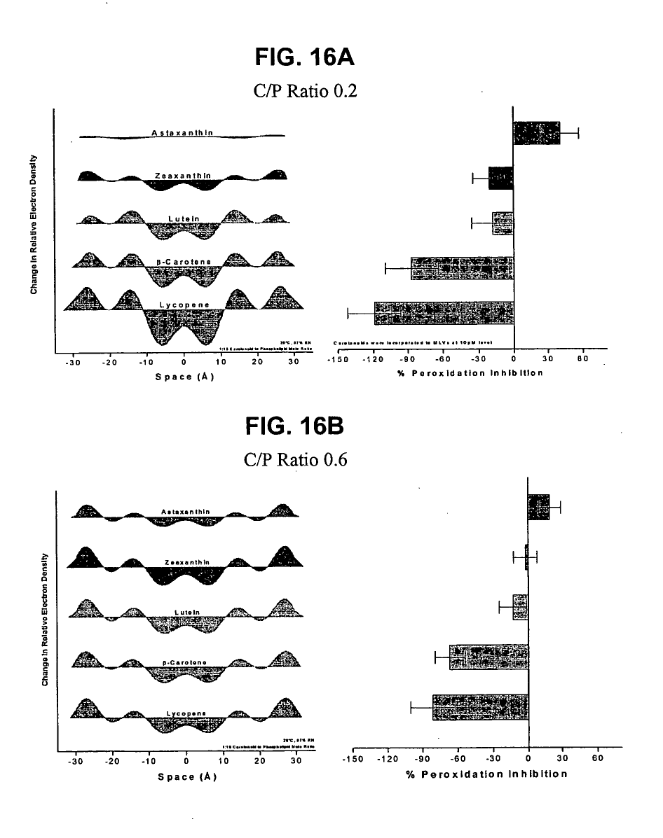

As clearly demonstrated in FIG. 16A, the pro-oxidant effects of carotenoids may be explained in light of modulation of membrane structure caused by carotenoids. Among all 5 carotenoids studied, only astaxanthin preserved the membrane structure while having a strong antioxidant effect. By contrast, the rest of the carotenoids studied exhibited disordering effects on membranes and pro-oxidant properties. In fact, a trend was noted in that the greater the disordering of membranes, the stronger the pro-oxidant effects of carotenoids.

Consistent with these findings, previous studies have indicated a link between rates of lipid peroxidation and membrane structure. It was observed that peroxidative damage to membranes may be modified by lipophilic drugs that have membrane ordering effects. In other reports, it has been proposed that vitamin E may have an important membrane-stabilizing effect which supplements its antioxidant activity. Other groups have also reported a direct effect of carotenoids on the physico-chemical properties of the membrane lipid bilayer. It has been shown, for example, that he addition of polar carotenoids to membranes alters its physical properties, especially membrane fluidity and permeability of small molecules (e.g., oxygen).

Consistent with these previous studies, we observed a correlation existed between membrane disordering and antioxidant effects of the carotenoids. We wanted to see if a similar correlation was exhibited in a more ordered membrane system. To achieve this condition, the cholesterol content in membranes was increased to the C/P ratio of 0.6 from 0.2 of the earlier study. With increased cholesterol content in membranes, variation in change of membrane structure caused by carotenoids was significantly diminished compared to its low cholesterol membrane counterpart (FIG. 15B). Carotenoids in the arthogenic membrane system (C/P 0.6) demonstrated a similar trend in antioxidant activities to the normal membrane (C/P 0.2), with lycopene having the highest prooxidant activity followed by β-carotene and astaxanthin being the only antioxidant (FIG. 16B).

In addition to its ability to increase membrane order, cholesterol is also known to promote peroxidation and change the drug partition coefficients in membranes. These factors might contribute to the lack of correlation between membrane disordering and antioxidant properties of the carotenoids observed in the more ordered membrane system.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

### REFERENCES

Bangham A D, Standish M M, Watkins J C. Diffusion of univalent ions across the lamellae of swollen phospholipids. J. Mol. Biol. 1965; 13:238-252.
Bolli R, Jeroudi M O, Patel B S, Aruoma O I, Halliwell B, Lai E K and McCay P B (1989) Marked reduction of free radical generation and contractile dysfunction by antioxidant therapy begun at the time of reperfusion. Evidence that myocardial “stunning” is a manifestation of reperfusion injury. Circ Res 65:607-622.
Britton G (1995) Structure and properties of carotenoids in relation to function. Faseb J 9:1551-1558.
Cantrell A, McGarvey D J, Truscott T G, Rancan F and Bohm F (2003) Singlet oxygen quenching by dietary carotenoids in a model membrane environment. Arch Biochem Biophys 412:47-54.
Cardounel A J, Dumitrescu C, Zweier J L and Lockwood S F (2003) Direct superoxide anion scavenging by a disodium disuccinate astaxanthin derivative: Relative efficacy of individual stereoisomers versus the statistical mixture of stereoisomers by electron paramagnetic resonance imaging. Biochem Biophys Res Commun 307:704-712.
Chung B H, Segrest J P, Ray M J, Brunzell J D, Hokanson J E, Krauss R M, Beaudrie K, Cone J T. Single vertical spin density gradient ultracentrifugation. Methods Enzymol. 1986; 128:181-208.
de Córdoba et al., “The Human Complement Factor H: Functional Roles, Genetic Variations and Disease Associations” Molecular Immunology Vol. 41, 2004, pp. 355-367
Curry S, Brick P and Franks N P (1999) Fatty acid binding to human serum albumin: new insights from crystallographic studies. Biochim Biophys Acta 1441:131-140.
de Beer F C, Hind C R, Fox K M, Allan R M, Maseri A and Pepys M B (1982) Measurement of serum C-reactive protein concentration in myocardial ischaemia and infarction. Br Heart J 47:239-243.
Devasagayam T P, Wemer T, Ippendorf H, Martin H D and Sies H (1992) Synthetic carotenoids, novel polyene polyketones and new capsorubin isomers as efficient quenchers of singlet molecular oxygen. Photochem Photobiol 55:511-514.
Diaz Padilla N, Bleeker W K, Lubbers Y, Rigter G M, Van Mierlo G J, Daha M R and Hack C E (2003) Rat C-reactive protein activates the autologous complement system. Immunology 109:564-571.
Edwards et al., “Complement Factor H Polymorphism and Age-Related Macular Degeneration” published online 10 Mar. 2005; 10.1126/science.1110189
El-Saadani, M., Esterbauer, H., El-Sayed, M., Goher, M., Nassar, A. Y., and Jurgens, G. (1989) A spectrophotometric assay for lipid peroxides in serum lipoproteins using a commercially available reagent. J. Lipid Research. 30(4), 627-630.
Esterbauer H, Striegl G, Puhl H, Rotheneder M. Continuous monitoring of in vitro oxidation of human low density lipoprotein. Free Radic. Res. Commun. 1989; 6:67-75.
Funkhouser T A and Vik D P (1999) Complement receptor type I gene regulation: retinoic acid and cytosine arabinoside increase CR1 expression. Scand J Immunol 49:21-28.
Gross G J and Lockwood S (In Press) Acute and chronic administration of disodium disuccinate astaxanthin (Cardax™) produces marked cardioprotection in dog hearts. Molecular and Cellular Biochemistry.
Gross G J and Lockwood S F (2004) Cardioprotection and myocardial salvage by a disodium disuccinate astaxanthin derivative (Cardax). Life Sci 75:215-224.
Gruszecki, W. I. (1999) in The photochemistry of carotenoids. Frank, H. A., Young, A. J., Britton, G., and Cogdell, R. J., Eds. pp. 363-379, Kluwer Academic, Dordrecht, The Netherlands.
Haines et al., “Complement Factor H Variant Increases the Risk of Age-Related Macular Degeneration” published online 10 Mar. 2005; 10.1126/science.1110359
Halliwell, B. (1997) Antioxidants and human disease: A general introduction. Nutr. Rev. 55, 544-552.
Huang D, Ou B, Hampsch-Woodill M, Flanagan J A, Prior R L. High-throughput assay of oxygen radical absorbance capacity (ORAC) using a multichannel liquid handling system coupled with a microplate fluorescence reader in 96-well format. J Agric Food Chem. 2002; 50:1815-1821.
Jensen S K, Engberg R M and Hedemann M S (1999) All-rac-alpha-tocopherol acetate is a better vitamin E source than all-rac-alpha-tocopherol succinate for broilers. J Nutr 129:1355-1360.
Jolly S R, Kane W J, Bailie M B, Abrams G D and Lucchesi B R (1984) Canine myocardial reperfusion injury. Its reduction by the combined administration of superoxide dismutase and catalase. Circ Res 54:277-285.
Kilgore K S, Friedrichs G S, Johnson C R, Schasteen C S, Riley D P, Weiss R H, Ryan U and Lucchesi B R (1994) Protective effects of the SOD-mimetic SC-52608 against ischemia/reperfusion damage in the rabbit isolated heart. J Mol Cell Cardiol 26:995-1006
Klein et al., “Complement Factor H Polymorphism in Age-Related Macular Degeneration” published online 10 Mar. 2005; 10.1126/science.1109557
Laemmli U K. Cleavage of structural proteins during the assembly of the head of bacteriophage. Nature. 1970; 227:680-686.
Lauver D A, Booth E A, White A J, Poradosu E and Lucchesi B R (2005) Sulodexide Attenuates Myocardial Ischemia/Reperfusion Injury and the Deposition of C-Reactive Protein in Areas of Infarction without Affecting Hemostasis. J Pharmacol Exp Ther 312:794-800.
Lawlor, S. M., and O'Brien, N. M. (1995) Astaxanthin:antioxidant effects in chicken embryo fibroblasts. Nutr. Res. 15, 1695-1704.
Lucchesi B R (1994) Complement, neutrophils and free radicals: mediators of reperfusion injury. Arzneimittelforschung 44:420-432.
Mathews-Roth, M. (1991) Recent progress in the medical applications of carotenoids. Pure Appl. Chem. 63, 147-156.
Mayne, S. T. (1996) Beta-carotene, carotenoids and disease prevention in humans. FASEB J. 10, 690-701
Mercadante, A. (1999) New carotenoids:recent progress. 12^{th} International Carotenoid Symposium, Cairns, Australia, July 1999.
Miki, W. (1991) Biological functions and activities of animal cartenoids. Pure Appl. Chem. 63, 141-146
Nijmeijer R, Lagrand W K, Lubbers Y T, Visser C A, Meijer C J, Niessen H W and Hack C E (2003) C-reactive protein activates complement in infarcted human myocardium. Am J Pathol 163:269-275.
Ong, A. S. H., and Tee. E. S. (1992) Natural sources of carotenoids from plants and oils. Meth. Enzymol. 213, 142-167.
Osterlie M, Bjerkeng B and Liaaen-Jensen S (2000) Plasma appearance and distribution of astaxanthin E/Z and R/S isomers in plasma lipoproteins of men after single dose administration of astaxanthin (1). J Nutr Biochem 11:482-490.
Palozza, P., and Krinsky, N. I. (1992) Astaxanthin and canthaxanthin are potent antioxidants in a membrane model. Arch. Biochem. Biophys. 297, 291-295.
Park J L and Lucchesi B R (1999) Mechanisms of myocardial reperfusion injury. Ann Thorac Surg 68:1905-1912.
Pascual M, Catana E, Spertini F, Macon K, Volanakis J E and Schifferli J A (1990) A monoclonal antibody which blocks the function of factor D of human complement. J Immunol Methods 127:263-269.
Peto, R., Doll, R., Buckley, J. D., and Sporn, M. B. (1981) Nature 290, 201-208.
Ridker et al. “C-Reactive Protein Levels and Outcomes after Statin Therapy” The New England Journal of Medicine, 2005, 352, pp 20-28.
Showalter L A, Weinman S A, Osterlie M and Lockwood S F (2004) Plasma appearance and tissue accumulation of non-esterified, free astaxanthin in C57BL/6 mice after oral dosing of a disodium disuccinate diester of astaxanthin (Heptax). Comp Biochem Physiol C Toxicol Pharmacol 137:227-236.
Simonyi M, Bikadi Z, Zsila F and Deli J (2003) Supramolecular exciton chirality of carotenoid aggregates. Chirality 15:680-698.
Subczynski, W. K., Markowska, E., and Sielewiesiuk, J. (1991) Effect of polar carotenoids on the oxygen diffusion-concentration product in lipid bilayers. An EPR spin label study. Biochim Biophys Acta 1068, 68-72.
Tanaka, T., Makita, H., Ohnishi, M., Mori, H., Satoh, K., and Hara, A. Chemoprevention of rat oral carcinogenesis by naturally occurring xanthophylls, astaxanthin and canthaxanthin. (1995) Cancer Res. 55, 4059-4064.
Tanhehco E J, Yasojima K, McGeer P L, Washington R A and Lucchesi B R (2000) Free radicals upregulate complement expression in rabbit isolated heart. Am J Physiol Heart Circ Physiol 279:H195-201.
Terao, J. (1989) Antioxidant activity of beta-carotene-related carotenoids in solution. Lipids 24, 659-661.
Venugopal et al. “Macrophage Conditioned Medium Induces the Expression of C-Reactive Protein in Human Aortic Endothelial Cells: Potential for Paracrine/Autocrine Effects” American Journal of Pathology, 2005, 166(4), pp 1265-1271.
Volanakis J E (1982) Complement activation by C-reactive protein complexes. Ann N Y Acad Sci 389:235-250.
Walter M F, Blumberg J B, Dolnikowski G G, Handelman G J. Streamlined F2-isoprostane analysis in plasma and urine with high-performance liquid chromatography and gas chromatography/mass spectroscopy. Anal. Biochem. 2000; 280:73-79.
Walter et al. “Sulfone COX-2 inhibitors increase susceptibility of human LDL and plasma to oxidative modification: comparison to sulfonamide COX-2 Inhibitors and NSAIDs”, Atherosclerosis 1 Vol. 77 (2004), pp. 235-243
Whaley K (1985) Methods in Complement for Clinical Immunologists. Churchill Livingstone, New York.
Yasojima K, Schwab C, McGeer E G and McGeer P L (1998) Human heart generates complement proteins that are upregulated and activated after myocardial infarction. Circ Res 83:860-869.
Yeh E T, Anderson H V, Pasceri V and Willerson J T (2001) C-reactive protein: linking inflammation to cardiovascular complications. Circulation 104:974-975.
Ytrehus K, Myklebust R, Olsen R and Mjos O D (1987) Ultrastructural changes induced in the isolated rat heart by enzymatically generated oxygen radicals. J Mol Cell Cardiol 19:379-389.
Ziegler, R. G., Mayne, S. T., and Swanson, C. A. (1996) Nutrition and lung cancer. Cancer Causes Control 7, 157-177.
Zsila F, Simonyi M and Lockwood S F (2003) Interaction of the disodium disuccinate derivative of meso-astaxanthin with human serum albumin: from chiral complexation to self-assembly. Bioorg Med Chem Lett 13:4093-4100.

1. A method of inhibiting, reducing or ameliorating at least some of the side effects associated with the administration of COX-2 inhibitors to a subject comprising administering to a subject that is receiving one or more COX-2 inhibitors, a therapeutically effective amount of a pharmaceutically acceptable composition comprising one or more carotenoids, carotenoid analogs, carotenoid derivatives, or combinations thereof.

2. The method of claim 1, wherein one or more of the carotenoids have the structure: 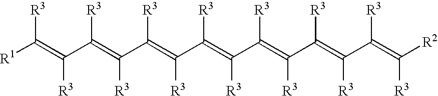
where each R³ is independently hydrogen or methyl, where each R¹ and R² are independently: 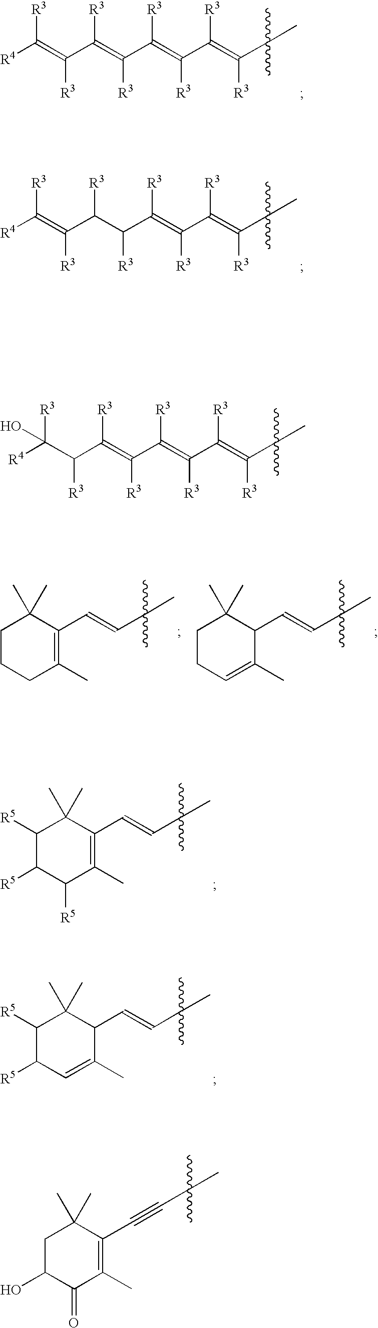 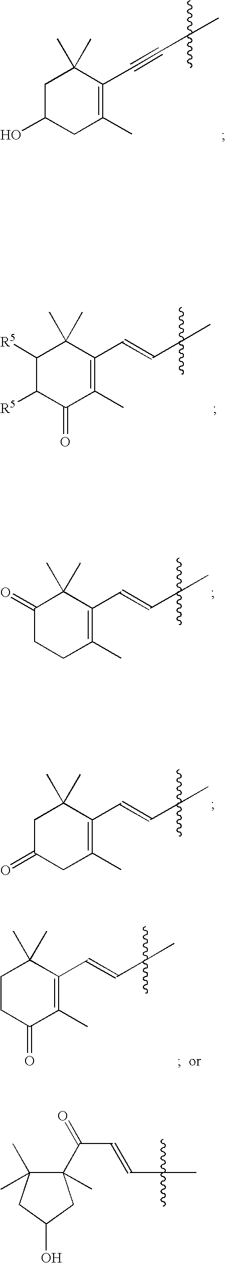
where R⁴ is hydrogen, methyl, or —CH₂OH; and where each R⁵ is independently hydrogen or —OH.

3. The method of claim 1, wherein one or more of the carotenoids have the structure: 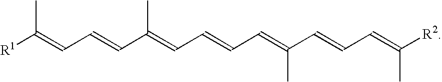
where each R¹ and R² are independently. 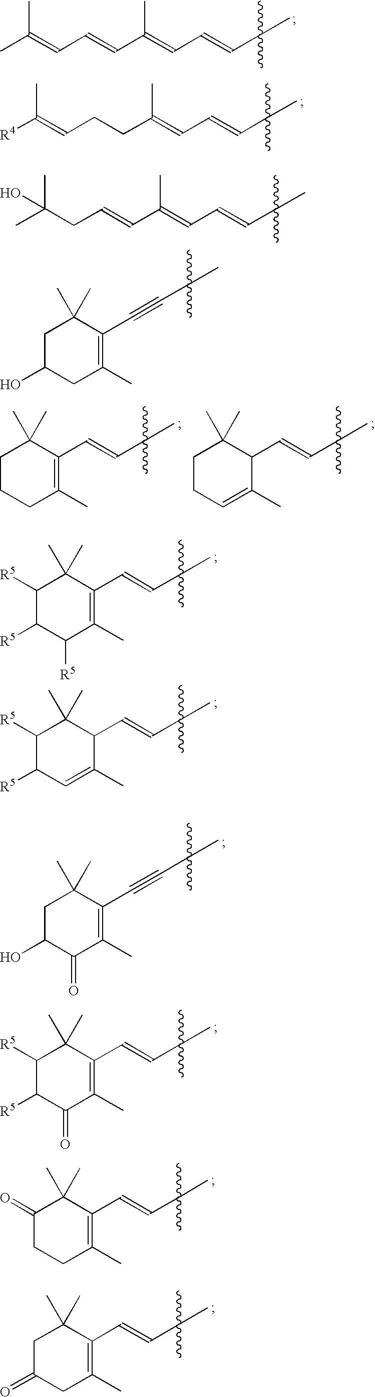 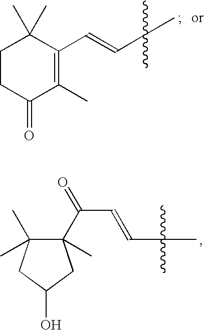
where R⁴ is hydrogen, methyl, or —CH₂OH; and where each R⁵ is independently hydrogen or —OH.

4. (canceled)

5. (canceled)

6. The method of claim 1, wherein one or more of the carotenoids have the structure: 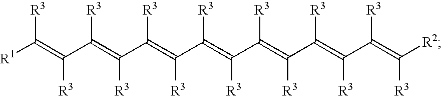
where each R³ is independently hydrogen or methyl, and where each R¹ and R² are independently: 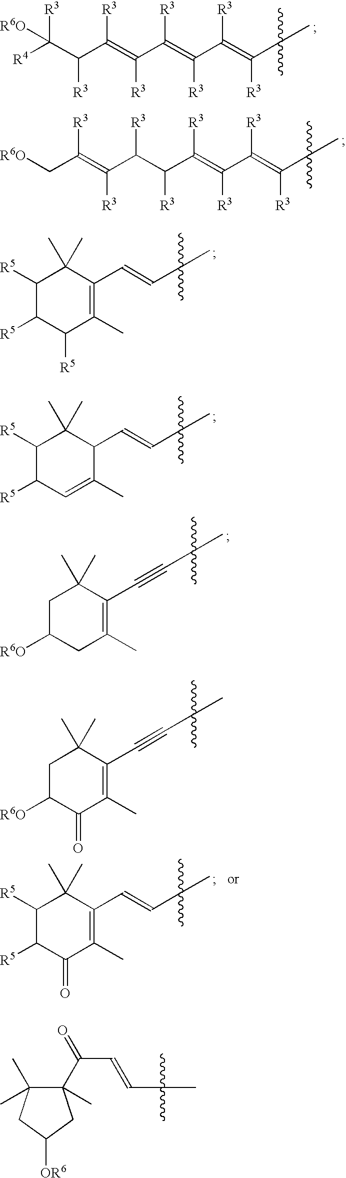
where R⁴ is hydrogen or methyl; where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸; —P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl or a con-antioxidant; where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

7. The method of claim 1, wherein one or more of the carotenoids have the structure: 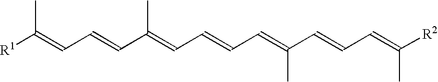
where each R¹ and R² are independently: 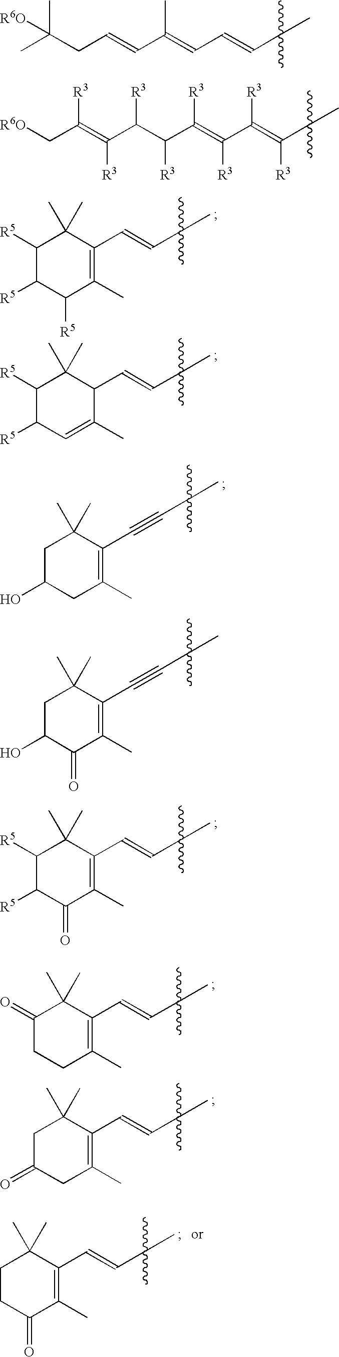 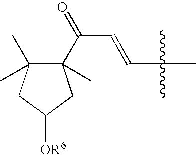
where each R⁵ is independently hydrogen, —OH, or —OR⁶ wherein at least one R⁵ group is —OR⁶; wherein each R⁶ is independently: alkyl; aryl; -alkyl-N(R⁷)₂; -aryl-N(R⁷)₂; -alkyl-CO₂H; -aryl-CO₂H; —O—C(O)—R⁸; P(O)(OR⁸)₂; —S(O)(OR⁸)₂; an amino acid; a peptide, a carbohydrate; —C(O)—(CH₂)ₙ—CO₂R⁹; a nucleoside reside, or a co-antioxidant; where R⁷ is hydrogen, alkyl, or aryl; wherein R⁸ is hydrogen, alkyl, aryl, benzyl, or a co-antioxidant; and where R⁹ is hydrogen; alkyl; aryl; —P(O)(OR⁸)₂; —S(O)(OR⁸)₂; an amino acid; a peptide, a carbohydrate; a nucleoside, or a co-antioxidant; and where n is 1 to 9.

8. The method of claim 7, wherein the substituent —OR⁶ comprises: 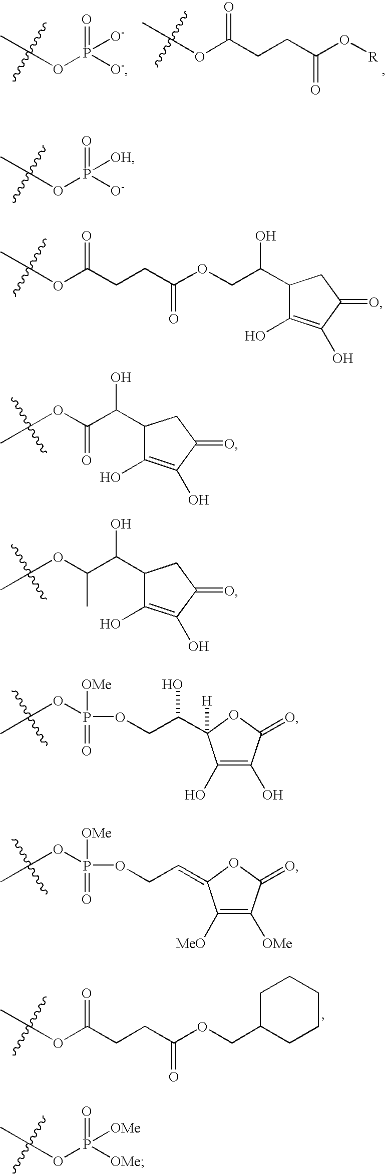
and wherein each R is independently H, alkyl, aryl, benzyl, Group IA metal, or co-antioxidant.

9. (canceled)

10. (canceled)

11. (canceled)

12. (canceled)

13. The method of claim 1, wherein one or more of the carotenoids have the structure: 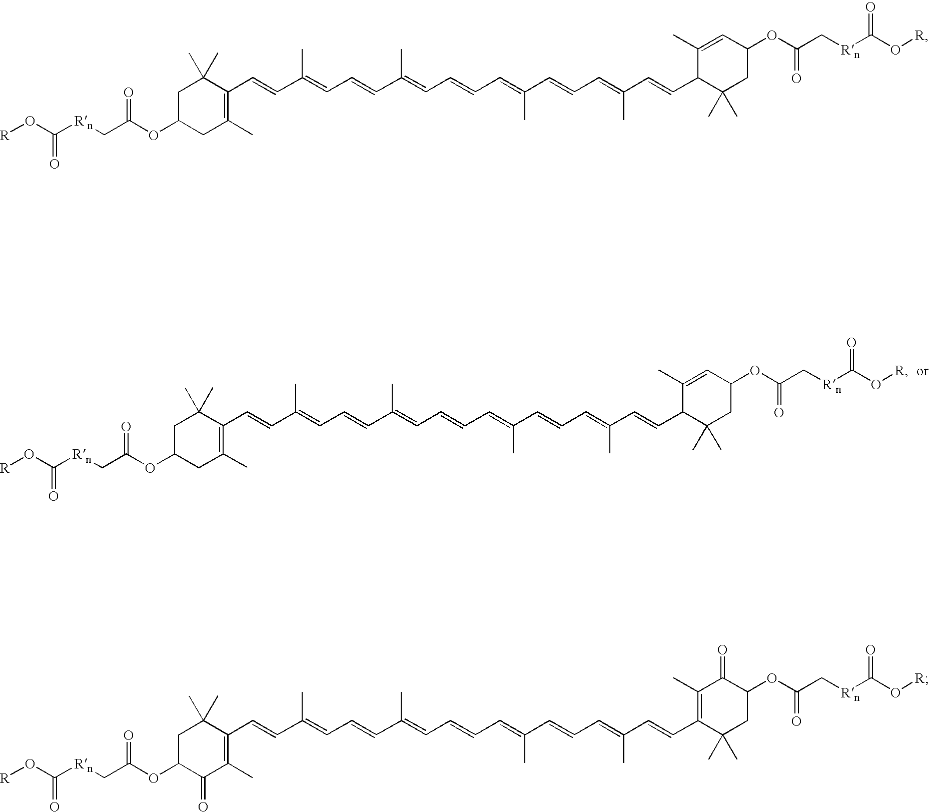
where each R is independently H, alkyl, aryl, benzyl, Group IA metal, or a co-antioxidant.

14. The method of claim 1, wherein one or more of the carotenoids have the structure: 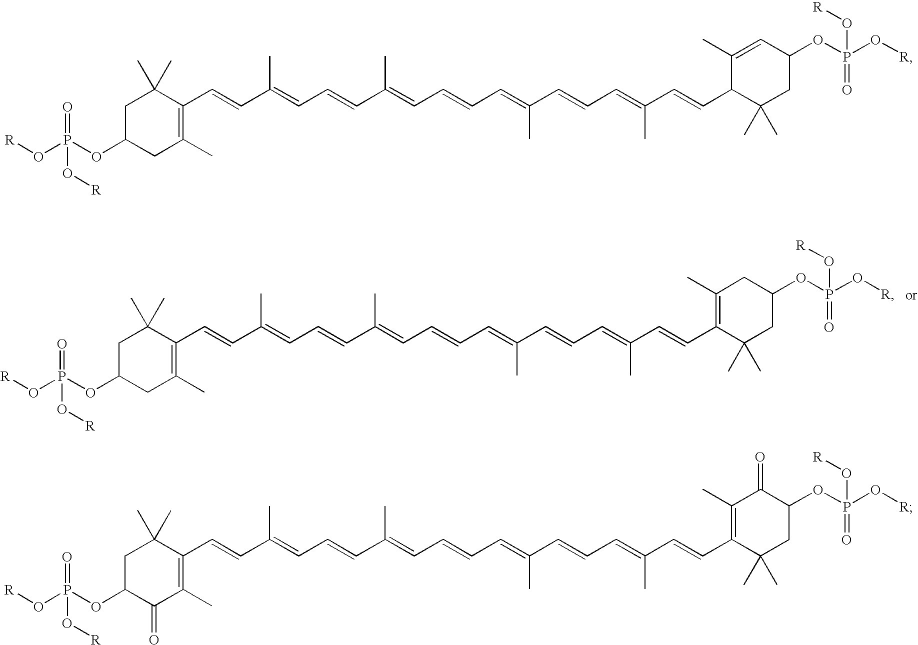
where each R is independently H, alkyl, aryl, benzyl, Group IA metal, or a co-antioxidant.

15. (canceled)

16. (canceled)

17. The method of claim 1, wherein one or more of the carotenoids have the structure: 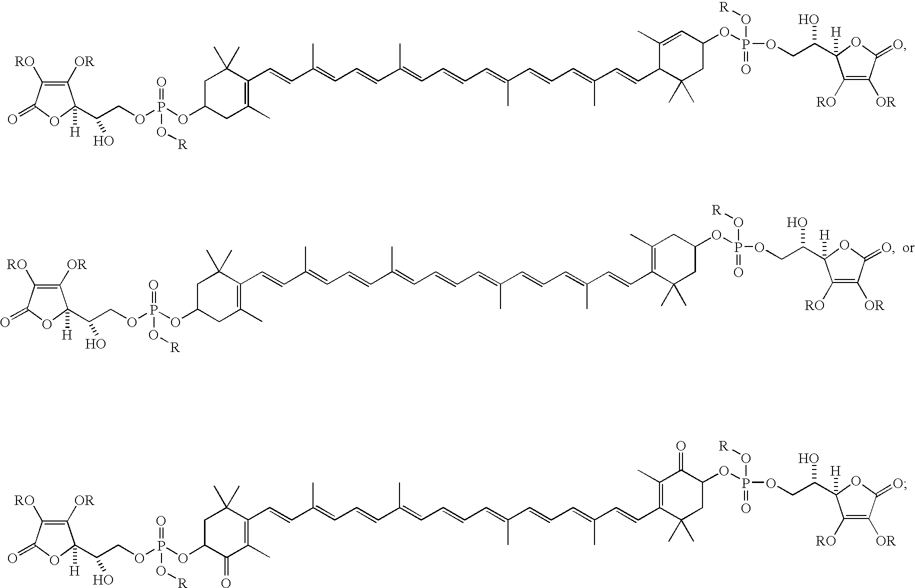
where each R is independently H, alkyl, aryl, benzyl, or a Group IA metal.

18. (canceled)

19. The method of claim 1, wherein the COX-2 inhibitor has the structure: 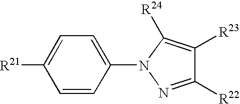
wherein R²¹ is selected from S(O)₂N(R²⁶)R²⁷, halo, alkyl, alkoxy, hydroxyl and haloalkyl;
wherein R²⁶ is hydrogen or alkoxycarbonylalkyl;
wherein R²⁷ is hydrogen, alkyl, carboxyalkyl, acyl, alkoxycarbonyl, heteroarylcarbonyl, alkoxycarbonylalkylcarbonyl, alkoxycarbonylcarbonyl, amino acid residue, or alkylcarbonylaminoalkylcarbonyl;
wherein R²² is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl and heterocyclic;
wherein R²³ is selected from hydrido, halo, haloalkyl, cyano, nitro, formyl, carboxyl, alkoxycarbonyl, carboxyalkyl, alkoxycarbonylalkyl, amidino, cyanoamidino, amido, alkoxy, amidoalkyl, N-monoalkylamido, N-monoarylamido, N,N-dialkylamido, N-alkyl-N-arylamido, alkylcarbonyl, alkylcarbonylalkyl, hydroxyalkyl, alkylthio, alkylsulfinyl, alkylsulfonyl, N-alkylsulfamyl, N-arylsulfamyl, arylsulfonyl, N,N-dialkylsulfamyl, N-alkyl-N-arylsulfamyl, heterocyclic, heterocycloalkyl and aralkyl;
wherein R⁷⁴ is selected from aryl, cycloalkyl, cycloalkenyl and heterocyclic; wherein R²⁴ is optionally substituted at a substitutable position with one or more radicals selected from halo, alkylthio, alkylsulfinyl, alkyl, alkylsulfonyl, cyano, carboxyl, alkoxycarbonyl, amido, N-monoalkylamido, N-monoarylamido, N,N-dialkylamido, N-alkyl-N-arylamido, haloalkyl, hydroxyl, alkoxy hydroxyalkyl haloalkoxy, sulfamyl, N-alkylsulfamyl, amino, N-alkylamino, N,N-dialkylamino, heterocyclic, nitro and acylamino; or wherein R³ and R²⁴ together form: 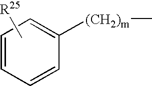
where m is 1 to 3, inclusive; and wherein R²⁵ is one or more radicals selected from halo, alkylthio, alkylsulfinyl, alkylsulfonyl, cyano, carboxyl, alkoxycarbonyl, amido, N-monoalkylamido, N-monoarylamido, alkyl, N,N-dialkylamido, N-alkyl-N-arylamido, haloalkyl, hydrido, hydroxyl, alkoxy, hydroxyalkyl, haloalkoxy, sulfamyl, N-alkylsulfamyl, amino, alkylamino, heterocyclic, nitro and acylamino; or a pharmaceutically-acceptable salt thereof.

20. The method of claim 1, wherein the COX-2 inhibitor has the structure: 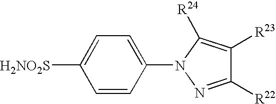
wherein R²² is haloalkyl; wherein R²³ is hydrido; and wherein R²⁴ is selected from aryl, cycloalkyl, and cycloalkenyl; wherein R²⁴ is optionally substituted at a substitutable position with one or more radicals selected from halo, alkylthio, alkylsulfonyl, cyano, nitro, haloalkyl, alkyl, hydrido, alkoxy, haloalkoxy, sulfamyl, heterocyclic and amino; or a pharmaceutically-acceptable salt thereof.

21. The method of claim 1, wherein the COX-2 inhibitor is celecoxib.

22. The method of claim 1, wherein the COX-2 inhibitor has the structure: 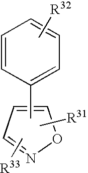
wherein R³¹ is selected from R—, RO—, RS—, RO-alkyl, RS-alkyl, carboxyl, cyano, hydroxyl, amino, halo, carboxyalkyl, alkoxycarbonylalkyl, aminocarbonyl, aminocarbonylalkyl, alkoxyalkyloxyalkyl, aryl(hydroxylalkyl), haloalkylsulfonyloxy, arylcarbonyloxyalkyl, arylcarbonylthioalkyl, alkoxycarbonyloxyalkyl, alkylaminocarbonyloxyalkyl, alkylaminocarbonylthioalkyl, RS(O)—; RS(O)alkyl-; RC(O)—; RC(O)alkyl-; ROC(O)—; ROC(O)alkyl-; RNH—; RNHalkyl-; R₂N—; R₂Nalkyl-; RS(O)₂alkyl-; and R^{a}O₂CR^{b}—X-alkyl-; wherein R is independently selected from alkyl, haloalkyl, hydroxyalkyl, aryl, cycloalkyl, heterocyclo, aralkyl, cycloalkylalkyl, and heterocycloalkyl; wherein R^{a} is selected from hydrido and R; wherein R^{b} is selected from a direct bond, alkyl, haloalkyl, hydroxyalkyl, aryl, cycloalkyl, heterocyclo, alkylaryl, aralkyl, cycloalkylalkyl, and heterocycloalkyl; wherein X is selected from O, S and S(O); wherein R³² is S(O)₂N(R³⁹)R⁴⁰; wherein R³⁹ is hydrogen or alkoxycarbonylalkyl; wherein R⁴⁰ is hydrogen, alkyl, carboxyalkyl, acyl, alkoxycarbonyl, heteroarylcarbonyl, alkoxycarbonylalkylcarbonyl, alkoxycarbonylcarbonyl, amino acid residue, or alkylcarbonylaminoalkylcarbonyl; and wherein R³³ is selected from cycloalkyl, cycloalkenyl, aryl and heterocyclo; wherein R³³ is optionally substituted at a substitutable position with one or more radicals independently selected from alkyl, cyano, carboxyl, alkoxycarbonyl, haloalkyl, hydroxyl, hydroxyalkyl, haloalkoxy, amino, alkylamino, arylamino, aminoalkyl, nitro, alkoxyalkyl, alkylsulfinyl, alkylsulfonyl, aminosulfonyl, halo, alkoxy and alkylthio; or a pharmaceutically-acceptable salt thereof.

23. The method of claim 1, wherein the COX-2 inhibitor has the structure: 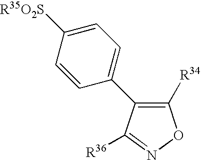
wherein R³⁴ is selected from hydroxyl, alkyl, carboxyl, halo, carboxyalkyl, alkoxycarbonylalkyl, aralkyl, methoxy, ethoxy, butoxy, alkylthio, alkoxyalkyl, aryloxyalkyl, arylthioalkyl, haloalkyl, hydroxylalkyl, aralkoxyalkyl, aryl(hydroxylalkyl), carboxyalkoxyalkyl, carboxyaryloxyalkyl, alkoxycarbonylaryloxyalkyl, cycloalkyl and cycloalkylalkyl; wherein R³⁵ is N(R³⁹)R⁴⁰; wherein R³⁹ is hydrogen; wherein R⁴⁰ is hydrogen, alkyl or —C(O)alkyl; and wherein R³⁶ is phenyl; wherein R³⁶ is optionally substituted at a substitutable position with one or more radicals independently selected from alkylsulfinyl, alkyl, cyano, carboxyl, alkoxycarbonyl, haloalkyl, hydroxyl, hydroxyalkyl, amino, haloalkoxy, alkylamino, phenylamino, aminoalkyl, nitro, halo, alkoxy, methylenedioxy, aminosulfonyl, and alkylthio; or a pharmaceutically-acceptable salt thereof.

24. The method of claim 1, wherein the COX-2 inhibitor has the structure: 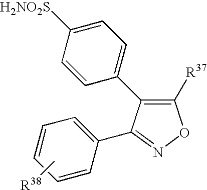
wherein R³⁷ is selected from hydroxyl, alkyl, carboxyl, halo, carboxyalkyl, alkoxycarbonylalkyl, alkoxyalkyl, carboxyalkoxyalkyl, haloalkyl, alkylthio, alkylsulfinyl, (hydroxy)alkoxyalkyl, carboxyalkylaryloxyalkyl, haloalkylsulfonyloxy, hydroxylalkyl, aryl(hydroxylalkyl), carboxyaryloxyalkyl, cycloalkyl, cycloalkylalkyl, and aralkyl; and wherein R³⁸ is one or more radicals independently selected from hydrido, alkylsulfinyl, alkyl, cyano, carboxyl, alkoxycarbonyl, haloalkyl, hydroxyl, hydroxyalkyl, haloalkoxy, amino, alkylamino, arylamino, aminoalkyl, nitro, halo, alkoxy, aminosulfonyl, and alkylthio; or a pharmaceutically-acceptable salt thereof.

25. The method of claim 1, wherein the COX-2 inhibitor is valdecoxib.

26. The method of claim 1, wherein the COX-2 inhibitor is parecoxib.

27. The method of claim 1, wherein the COX-2 inhibitor has the structure: 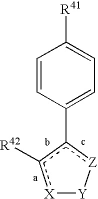
wherein: X-Y-Z-is: (a) —CH₂CH₂CH₂—; (b) —C(O)CH₂CH₂—; (c) —CH₂CH₂C(O)—;
(d) —CR⁴⁵(R⁴⁵)—O—C(O)—; (e) —C(O)—O—CR⁴⁵(R^{45′})—; (f) —CH₂—NR₃—CH₂—;
(g) —CR⁴⁵(R^{45′})—NR₃—C(O)—; (h) —CR⁴⁴═CR^{44′}—S—; (i) —S—CR⁴⁴═CR^{44′}—; (j) —S—N═CH—;
(k) —CH═N—S—; (l) —N═CR⁴⁴—O—; (m) —O—CR⁴⁴═N—; (n) —N═CR⁴⁴—N—H—;
(o) —N═CR⁴⁴—S—; (p) —S—CR⁴⁴—N—; (q) —C(O)—NR⁴³—CR⁴⁵(R^{45′})—; (r) —R⁴³N—CH═CH—; or
(s) —CH═CH—NR⁴³— when side b is a double bond, and sides a an c are single bonds; and
X-Y-Z-is: (a) ═CH—O—CH═; (b) ═CH—NR⁴³—CH═; (c) ═N—S—CH═; (d) ═CH—S—N═;
(e) ═N—O—CH═; (f) ═CH—O—N═; (g) ═N—S—N═; or (h)═N—O—N═ when sides a and c are double bonds and side b is a single bond;
where R⁴¹ is: (a) S(O)₂CH₃; (b) S(O)₂NH₂; (c) S(O)₂NHC(O)CF₃; (d) S(O)(NH)CH₃;
(e) S(O)(NH)NH₂; (f) S(O)(NH)NHC(O)CF₃; (g) P(O)(CH₃)OH; (h) P(O)(CH₃)NH₂; (i) S(O)₂NH-alkyl;
(j) S(O)₂NH-aryl; (k) S(O)₂NHC(O)-alkyl; or (l) S(O)₂NHC(O)aryl;
where R⁴² is (a) C₁₋₆ alkyl; (b) C₃, C₄, C₅, C₆, or C₇ cycloalkyl; (c) mono-, di- or tri-substituted phenyl or naphthyl wherein possible substituents include hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R⁴⁵)(R⁴⁶)—OH, —C(R⁴⁵)(R⁴⁶)—O—C₁₋₄ alkyl, and (13) —C₁₋₆ alkyl-CO₂—R⁴⁵; (d) mono-, di- or tri-substituted heteroaryl wherein the heteroaryl is a monocyclic aromatic ring of 5 atoms, the monocyclic ring having one heteroatom which is S, O, or N, and optionally 1, 2, or 3 additionally N atoms; or the heteroaryl is a monocyclic ring of 6 atoms, the monocyclic ring having one heteroatom which is N, and optionally 1, 2, 3, or 4 additional N atoms; wherein possible substituents include hydrogen, halo (including fluoro, chloro, bromo and iodo), C₁₋₆ alkyl, C₁₋₆ alkoxy, C₁₋₆ alkylthio, CN, CF₃, N₃, —C(R⁴⁵)(R⁴⁶)—OH, and —C(R⁴⁵)(R⁴⁶)—O—C₁₋₄ alkyl; or (e) benzoheteroaryl which includes the benzo fused analogs of (d);
where each R⁴³ is: (a) hydrogen; (b) CF₃; (c) CN; (d) C₁₋₆ alkyl; (e) hydroxy C₁₋₆ alkyl;
(f) —C(O)—C₁₋₆ alkyl; (g) optionally substituted (1) —C₁₋₅ alkyl-Q,
(2) —C₁₋₃ alkyl-O—C₁₋₃ alkyl-Q, (3) —C₁₋₃ alkyl-S—C₁₋₃ alkyl-Q,
(4) —C₁₋₅ alkyl-O-Q, or (5) —C₁₋₅ alkyl-S-Q, wherein the substituent resides on the alkyl and the substituent is C₁₋₃ alkyl; or (h) -Q
where R⁴⁴ and R^{44′} are each independently: (a) hydrogen; (b) CF₃; (c) CN; (d) C₁₋₆ alkyl; (e) -Q; (f) —O-Q; (g) —S-Q, or (h) optionally substituted (1) —C₁₋₅ alkyl-Q, (2) —O—C₁₋₅ alkyl-Q, (3) —S—C₁₋₅ alkyl-Q, (4) —C₁₋₃ alkyl-O—C₁₋₃ alkyl-Q, (5) —C₁₋₃ alkyl-S—C₁₋₃ alkyl-Q, (6) —C₁₋₅ alkyl-O-Q, (7) —C₁₋₅ alkyl-S-Q, wherein the substituent resides on the alkyl and the substituent is C₁₋₃ alkyl, and where R⁴⁵, R^{45′}, R⁴⁶, R⁴⁷ and R⁴⁸ are each independently: (a) hydrogen; (b) C₁₋₆ alkyl; or R⁴⁵ and R⁴⁶ or R⁴⁷ and R⁴⁸ together with the carbon to which they are attached form a saturated monocyclic carbon ring of 3, 4, 5, 6 or 7 atoms;
where Q is CO₂H, CO₂—C₁₋₄ alkyl, tetrazolyl-5-yl, C(R⁴⁷)(R⁴⁸)(OH), or C(R⁴⁷)(R⁴⁸)(O—C₁₋₄ alkyl).

28. The method of claim 1, wherein the COX-2 inhibitor has the structure: 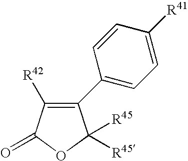
where R⁴¹ is: (a) S(O)₂CH₃; (b) S(O)₂NH₂; (c) S(O)₂NHC(O)CF₃; (d) S(O)(NH)CH₃;
(e) S(O)(NH)NH₂; (f) S(O)(NH)NHC(O)CF₃; (g) P(O)(CH₃)OH; (h) P(O)(CH₃)NH₂; (i) S(O)₂NH-alkyl;
(j) S(O)₂NH-aryl; (k) S(O)₂NHC(O)-alkyl; or (l) S(O)₂NHC(O)aryl;
where R⁴² is (a) C₁₋₆ alkyl; (b) C₃, C₄, C₅, C₆, or C₇ cycloalkyl; (c) mono-, di- or tri-substituted phenyl or naphthyl wherein possible substituents include hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R⁴⁵)(R⁴⁶)—OH, —C(R⁴⁵)(R⁴⁶)—O—C₁₋₄ alkyl, and (13) —C₁₋₆ alkyl-CO₂—R⁴⁵; (d) mono-, di- or tri-substituted heteroaryl wherein the heteroaryl is a monocyclic aromatic ring of 5 atoms, the monocyclic ring having one heteroatom which is S, O, or N, and optionally 1, 2, or 3 additionally N atoms; or the heteroaryl is a monocyclic ring of 6 atoms, the monocyclic ring having one heteroatom which is N, and optionally 1, 2, or 3 additional N atoms; wherein possible substituents include hydrogen, halo (including fluoro, chloro, bromo and iodo), C₁₋₆ alkyl, C₁₋₆ alkoxy, C₁₋₆ alkylthio, CN, CF₃, N₃, —C(R⁴⁵)(R⁴⁶)—OH, and —C(R⁴⁵)(R⁴⁶)—O—C₁₋₄ alkyl; and
where R⁴⁵ and R⁴⁶ are each independently: (a) hydrogen; (b) C₁₋₆ alkyl; or R⁴⁵ and R⁴⁶ together with the carbon to which they are attached form a saturated monocyclic carbon ring of 3, 4, 5, 6 or 7 atoms.

29. The method of claim 1, wherein the COX-2 inhibitor has the structure: 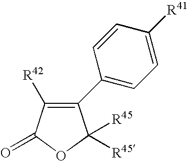
where R⁴¹ is S(O)₂CH₃, S(O)₂NH₂, S(O)NHCH₃, S(O)NHNH₂, S(O)₂NH-alkyl, S(O)₂NH-aryl, S(O)₂NHC(O)-alkyl, or S(O)₂NHC(O)aryl;
where R⁴² is C₁₋₆ alkyl; C₃, C₄, C₅, C₆, and C₇, cycloalkyl; (c) heteroaryl
(d) benzoheteroaryl (e) mono- or di-substituted phenyl wherein possible substituents include hydrogen, halo, C₁₋₆ alkoxy, C₁₋₆ alkylthio, CN, CF₃, C₁₋₆ alkyl, N₃, —CO₂H, —CO₂—C₁₋₄ alkyl, —C(R⁴⁵)(R⁴⁶)—OH, —C(R⁴⁵)(R⁴⁶)—O—C alkyl, or C₁₋₆ alkyl-CO₂—R⁴⁵;
R⁴⁵, R^{45′} and R⁴⁶ are each independently (a) hydrogen; (b) C₁₋₆ alkyl; or R⁴⁵ and R⁴⁶ together with the carbon to which they are attached form a saturated monocyclic carbon ring of 3, 4, 5, 6 or 7 atoms.

30. The method of claim 1, wherein the COX-2 inhibitor is rofecoxib.

31. The method of claim 1, wherein the COX-2 inhibitor has the structure: 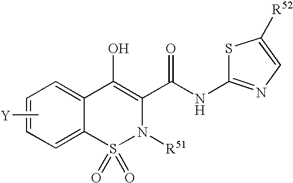
where R⁵¹ is hydrogen, methyl or ethyl;
where R⁵² is methyl, ethyl or n-propyl; and
where Y is hydrogen, methyl, methoxy, fluorine or chlorine.

32. The method of claim 1, wherein the COX-2 inhibitor is meloxicam.

33. The method of claim 1, wherein the COX-2 inhibitor has the structure: 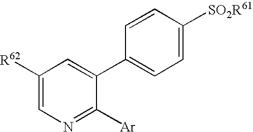
where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where Ar is a mono-, di-, or trisubstituted phenyl or pyridinyl (or the N-oxide thereof), wherein substituents include hydrogen, halogen, C₁₋₆ alkoxy, C₁₋₆ alkylthio, CN,
C₁₋₆ alkyl, C₁₋₆ fluoroalkyl, N₃, —CO₂R⁶³, hydroxy, —C(R⁶⁴)(R⁶⁵)—OH, —C₁₋₆ alkyl-CO₂—R⁶⁶, or C₁₋₆ fluoroalkoxy;
where R⁶² is: (a) halo; (b) C₁₋₆ alkoxy; (c) C₁₋₆ alkylthio; (d) CN; (e) C₁₋₆ alkyl;
(f) C₁₋₆ fluoroalkyl; (g) N₃; (h) —CO₂R⁶⁷; (i) hydroxy; (j)—C(R⁶⁸)(R⁶⁹)—OH;
(k) —C₁₋₆ alkyl-CO₂—R⁷⁰; (l) C₁₋₆ fluoroalkoxy; (m) NO₂; (n) NR⁵³R⁵⁴; and
(o) NHCOR⁵⁵; and
where R⁶³, R⁶⁴, R⁶, R⁶⁶, R⁶⁷, R⁶⁸, R⁶⁹, R⁷⁰, R⁵³, R⁵⁴, R⁵⁵, are each independently hydrogen or C₁₋₆ alkyl, or R⁶⁴ and R⁶⁵, R⁶⁸ and R⁶⁹ or R⁵³ and R⁵⁴ together with the atom to which they are attached form a saturated monocyclic ring of 3, 4, 5, 6 or 7 atoms.

34. The method of claim 1, wherein the COX-2 inhibitor has the structure: 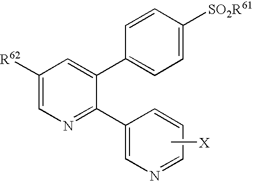
where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where R⁶² is: (a) halo; (b) C₁₋₃ alkoxy; (c) C₁₋₃ alkylthio; (d) C₁₋₃ alkyl; (e) N₃; (f) —CO₂H;
(g) hydroxy; (h) C₁₋₃ fluoroalkoxy; (i) NO₂; (j) NR⁵³R⁵⁴ and (k) NHCOR⁵⁵; and
where X is methyl, ethyl, n-propyl, i-propyl or cyclopropyl.

35. The method of claim 1, wherein the COX-2 inhibitor has the structure: 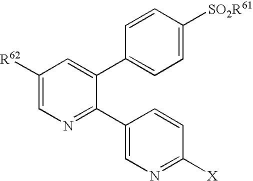
where R⁶¹ is: (a) CH₃; (b) NH₂; (c) NHC(O)CF₃; (d) NH-alkyl; (e) NH-aryl; (f) NHC(O)-alkyl; or (g) S(O)₂NHC(O)aryl;
where R⁶² is chloro or methyl;
and where there may be one, two or three X groups, where each X group is independently: hydrogen, halogen, C₁₋₄ alkoxy, C₁₋₄ alkylthio, CN, C₁₋₄ alkyl, or CF₃.

36. The method of claim 1, wherein the COX-2 inhibitor is etoricoxib.

37. The method of claim 1, wherein the COX-2 inhibitor has the structure: 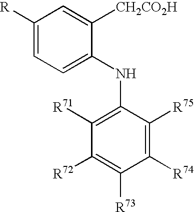
R is methyl or ethyl; R⁷¹ is chloro or fluoro; R⁷² is hydrogen or fluoro; R⁷³ is hydrogen, fluoro, chloro, methyl, ethyl, methoxy, ethoxy or hydroxy; R⁷⁴ is hydrogen or fluoro; and R⁷⁵ is chloro, fluoro, trifluoromethyl or methyl; pharmaceutically acceptable salts thereof; and pharmaceutically acceptable prodrug esters thereof.

38. The method of claim 1, wherein the COX-2 inhibitor is lumiracoxib.

39. The method of claim 1, wherein the formulation comprising the carotenoid, the carotenoid analog or the carotenoid derivative is administered to the subject prior to the commencement of COX-2 inhibitor drug therapy.

40. The method of claim 1, wherein the formulation comprising the carotenoid, the carotenoid analog or the carotenoid derivative is administered to the subject concurrently with the COX-2 inhibitor drug therapy.

41-54. (canceled)

55. A composition comprising one or more COX-2 inhibitors and one or more carotenoids, carotenoid analogs, carotenoid derivatives, or combinations thereof.

56-91. (canceled)

92. A pharmaceutical composition comprising: one or more COX-2 inhibitors, one or more carotenoids, carotenoid analogs; and carotenoid derivatives, or combinations thereof; and a biologically inactive carrier; wherein the pharmaceutical composition is adapted to be administered to a human subject.

93-98. (canceled)
